(19) United States
(12) Patent Application Publication
(10) Pub. No.: US 2022/0322956 A1
(43) Pub. Date: Oct. 13, 2022

(54) SYSTEM AND METHOD FOR REMOTE ASSESSMENT AND CORRECTION OF BASELINE PRESSURE INSTABILITY OF MEDICAL PRESSURE SENSORS

(71) Applicant: dPCom A/S, Oslo (NO)

(72) Inventor: Per Kristian Eide, Oslo (NO)

(73) Assignee: dPCom A/S, Oslo (NO)

(21) Appl. No.: 17/843,856

(22) Filed: Jun. 17, 2022

Related U.S. Application Data
(63) Continuation-in-part of application No. PCT/NO2021/050036, filed on Feb. 5, 2021.

(30) Foreign Application Priority Data
Feb. 15, 2020 (NO) 20200199
Feb. 15, 2020 (NO) 20200200
Feb. 15, 2020 (NO) 20200201
Feb. 15, 2020 (NO) 20200202
Feb. 15, 2020 (NO) 20200203

(51) Int. Cl. A61B 5/03; A61B 5/00

(52) U.S. Cl. CPC A61B 5/036; A61B 5/7221; A61B 5/0002; A61B 2562/0247; A61B 2562/04

(57) ABSTRACT

Described herein are systems, devices, and methods for remote assessment and correction of baseline pressure instability of medical pressure sensors. The present disclosure enables centralized surveillance of baseline pressure instability, which provides a technical solution for manufacturers of pressure sensors and/or pressure transducer systems to monitor the proper function of their products. Moreover, health care personnel are provided with means to assess pressure sensor instability and get corrected pressure scores, neither of which are presently unavailable. The disclosure thereby provides a technical solution to a problem of medical pressure sensors that represents risk of harm to patients.

### CROSS-REFERENCE TO RELATED APPLICATIONS

This application is a continuation-in-part of International Application No. PCT/NO 2021/050036, which claims the priority benefit of Norweigian App. Nos. 20200199, 20200200, 20200201, 20200202, and 20200203, each filed on Feb. 15, 2020, each of which are incorporated by reference herein in their entireties.

### BACKGROUND

### Field

Embodiments of the present disclosure relate to systems, methods, and devices for addressing baseline pressure instability of pressure sensors for measuring pressures within a human body cavity such as the cranio-spinal cavity or a blood vessel compartment. Embodiments of the present disclosure particularly address measurements of intracranial pressure (ICP), arterial blood pressure (ABP), as well as pressure indices derived from ICP and ABP, such as cerebral perfusion pressure (CPP).

### Background

Invasive intracranial pressure (ICP) monitoring has an important role in the diagnosis and surveillance of patients with various types of brain damage or brain disease. For surveillance of patients with brain damage, e.g., due to trauma, stroke or as a complication to brain surgery, usually the ICP is measured together with arterial blood pressure (ABP). The so-called cerebral perfusion pressure (CPP) is computed according to this formula: mean CPP=mean ABP—mean ICP, and is an important parameter for patient surveillance. The common treatment goals are to keep ICP <20 mmHg and CPP >50-60 mmHg. This is done to avoid compromised blood flow to the brain, which is the main source of energy delivery to brain cells. Since the cranium is rigid without ability to expand (e.g., after about 2 years age), any disease process increasing the volume of intracranial components may cause increased ICP, which may hamper blood flow to the brain. Thus, monitoring of ICP and ABP may be crucial, such as by methods of invasive monitoring of human pressures using pressure sensors.

Conventionally, the mean ICP and mean ABP are measured relative to a baseline (or a reference) pressure value. ICP and ABP measurements depend on a stable baseline pressure. However, inherent properties of pressure sensors and current measurement technology may affect the baseline pressure, causing the baseline pressure to vary spontaneously during ongoing in vivo measurements, and resulting in baseline pressure instability. The instability of baseline pressure of pressure sensors may cause erroneous pressure measurements. Incorrect ICP and ABP readings may ultimately lead to erroneous patient management.

### BRIEF SUMMARY

Accordingly, there may be a need for technical solutions for solving issues related to measurements of pressures within a human body cavity such as the cranio-spinal cavity or a blood vessel compartment.

A first aspect of the disclosure describes means for assessing pressure sensor instability and related baseline pressure instability of a pressure sensor applied for sampling of continuous pressure signals originating from inside a human body or body cavity. Issuance of an alert is enabled if the stability deviates from set thresholds. This aspect evolved from the need of developing technical solutions for assessing baseline pressure stability. From measurements of mean ICP and mean ABP in humans, it may be found that currently used pressure sensors are prone to baseline pressure instability. Further, laboratory testing of ICP sensors revealed that currently used pressure sensors are sensitive to electrostatic discharges, which may be one cause of baseline pressure instability. From these observations, it was identified that indicators of stability of baseline pressure should be developed. Currently, end-users of pressure-monitoring equipment, i.e. physicians, nurses and other health care personnel, are not alarmed about baseline pressure instability, which may be dangerous for patient management.

A feature of the disclosure includes a method for assessing stability of baseline pressure of a pressure sensor applied for sampling of continuous pressure signals originating from inside a human body or body cavity, samples of the pressure signals from the sensor being obtainable at specific intervals, and being convertible into pressure-related digital data with a time reference, the method comprising:

from the digital data identification of single pressure waves related to cardiac beat-induced pressure waves,

detection of single pressure wave (SW.x)-related parameters selectable from one or more of mean pressure (SW.meanP) and amplitude (SW.dP), and

computation of delta single pressure wave (dSW.x)-related parameters, representing differences in single pressure wave (dSW.x)-related parameters selectable from one or more of change in mean pressure (dSW.meanP), and change in amplitude (dSW.dP) between a consecutive number of single pressure waves (n−1; n),

wherein calculation of pressure stability levels (SW.x.PSL) of the single pressure wave (SW.x)-related parameters is created from consecutive single pressure waves having any one of delta single pressure wave (dSW.x)-related parameters dSW.meanP and dSW.dP within a first type of selectable thresholds, the first type of thresholds referring to defined pressure ranges of any one of the parameters dSW.meanP and dSW.dP, and wherein a pressure stability level refers to average of any one of the single pressure wave (SW.x)-related parameters SW.meanP and SW.dP,

wherein determination is made of pressure differences between different of the pressure stability levels (n−1; n) (SW.x.PSL.PD),

the pressure stability levels (SW.x.PSL) having definable time durations (SW.x.PSL.TD) relating to the time duration of the pressure stability levels (SW.x.PSL),

and the pressure stability levels of definable durations (SW.x.PSL.TD) and with beginning and ending pressure differences (SW.x.PSL.PD) for each pressure stability level (SW.x.PSL) together creating a baseline pressure indicator (BPi) plot, the beginning pressure difference being defined as the difference between a present and a previous pressure stability level and the ending pressure difference being defined as the difference between a present and a next pressure stability level,

the plot providing information about stability of baseline pressure of the pressure sensor and being a function of at least one of:

a) combinations of pressure differences between different of the pressure stability levels (SW.x.PSL), calculated from the same type of single pressure wave (SW.x)-related parameters, the pressure differences being outside or inside a second type of selectable set thresholds, reflecting deviations from nominal reference pressure differences, and

b) relationships between different and simultaneous pressure stability levels (n−1; n) calculated from different types of single pressure wave (SW.x)-related parameters, the relationships being outside or inside a third type of selectable set thresholds, reflecting deviations from nominal reference relationships, and

wherein parameters of a) and/or b) outside the second set and/or the third set of thresholds thereby define instability of baseline pressure of the pressure sensor.

A feature of the disclosure includes a system for assessing stability of baseline pressure of a pressure sensor applied for sampling of continuous pressure signals originating from inside a human body or body cavity, wherein the system comprises:

a pressure sensor configured to measure pressure signals from the human body or body cavity at specific intervals;

a transfer means configured to transfer the pressure signals from the pressure sensor to a sampling unit;

a signal converter in communication with the sampling unit and configured to perform conversion of sampled pressure signals, from the sampling unit, into pressure-related digital data with a time reference;

an identifier unit configured to receive the pressure-related digital data from the signal converter and identify therefrom single pressure waves related to cardiac beat-induced pressure waves;

a detector connected to an output of the identifier unit and configured to detect single pressure wave (SW.x)-related parameters, being one or more of single wave mean pressure (SW.meanP), and single wave amplitude (SW.dP); and

a computing device connected to an output of the detector and configured to compute one or more of delta single pressure wave (dSW.x)-related parameters representing differences in single pressure wave (dSW.x)-related parameters being one or more of change in mean pressure (dSW.meanP), and change in amplitude (dSW.dP), between a consecutive number of single pressure waves (n−1;n),

wherein a calculation unit is connected to an output of the computing device and configured to calculate pressure stability levels (SW.x.PSL), each pressure stability level being created from consecutive single pressure waves having any one of the delta single pressure wave (dSW.x)-related parameters dSW.meanP and dSW.dP within a first set of thresholds, the first set of thresholds referring to defined pressure ranges of any one of the parameters dSW.meanP and dSW.dP, wherein each pressure stability level refers to an average of any one of the single pressure wave (SW.x)-related parameters SW.meanP and SW.dP,

wherein a determination unit is connected to an output of the calculation unit and configured to determine pressure differences (SW.x.PSL.PD) between different pressure stability levels (n−1;n) (SW.x.PSL),

wherein the pressure stability levels (SW.x.PSL) have definable time durations (SW.x.PSL.TD) relating to a time duration of the pressure stability levels (SW.x.PSL),

wherein a presentation unit is connected to an output of the determination unit and configured to present baseline pressure indicator (BPi) plots, being created from the pressure stability levels (SW.x.PSL) and with beginning pressure differences and ending pressure differences (SW.x.PSL.PD) for each pressure stability level (SW.x.PSL), the beginning pressure difference being defined as a difference between a present pressure stability level and a previous pressure stability level and the ending pressure difference being defined as a difference between a present pressure stability level and a next pressure stability level,

wherein the BPi plots provide information about stability of baseline pressure of the pressure sensor and are a function of at least one of:

a) combinations of the pressure differences between different pressure stability levels (SW.x.PSL), calculated from a same type of single pressure wave (SW.x)-related parameters, the pressure differences being outside or inside a second set of thresholds, reflecting deviations from nominal reference pressure differences, and

b) relationships between different and simultaneous pressure stability levels (n−1; n) calculated from different types of single pressure wave (SW.x)-related parameters, the relationships being outside or inside a third set of thresholds, reflecting deviations from nominal reference relationships, and

wherein the presentation unit is configured to indicate if parameters of a) and/or b) are outside the second set and/or the third set of thresholds and thereby define instability of baseline pressure of the pressure sensor.

A feature of the disclosure includes a system for assessing intracranial pressure (ICP) in a human, the system comprising:

a pressure sensor that is insertable into a cranio-spinal cavity or in communication with fluid of the cranio-spinal cavity, the pressure sensor being configured to measure ICP signals, which represent differences in pressure between atmospheric pressure and pressure inside the cranio-spinal cavity; and

a pressure analyzer unit in communication with the pressure sensor, the pressure analyzer unit being configured to:
process and analyze the ICP signals from the pressure sensor;
based on the processing and analyzing of the ICP signals, provide one or more baseline pressure indicator (BPi) plots created from pressure stability levels (SW.x.PSL) of definable time durations (SW.x.PSL.TD), calculated from single pressure wave (SW.x)-related parameters from a predefined number of single pressure waves having delta single pressure wave (dSW.x)-related parameters within a first set thresholds, the first set of thresholds referring to defined pressure ranges of any one of parameters dSW.meanP and dSW.dP, and with beginning pressure differences and ending pressure differences for each pressure stability level (SW.x.PSL.PD), the beginning pressure difference being defined as a difference between a present pressure stability level and a previous pressure stability level, and the ending pressure difference being defined as a difference between a present pressure stability level and a next pressure stability level,

wherein the pressure analyzer unit has an outlet and an information provider device configured to provide information about the stability of baseline pressure of the pressure sensor from the BPi plot, the information being a function of at least one of:

a) combinations of pressure differences between different pressure stability levels (SW.x.PSL), calculated from a same type of single pressure wave (SW.x)-related parameters, the pressure differences being outside or inside a second set of thresholds, reflecting deviations from nominal reference pressure differences, and

b) relationships between different and simultaneous pressure stability levels (n−1; n) calculated from different types of single pressure wave (SW.x)-related parameters, the relationships being outside or inside a third set of thresholds, reflecting deviations from nominal reference relationships, wherein parameters of a) and/or b) outside the second and/or the third sets of thresholds define instability of baseline pressure of the pressure sensor, and

wherein the information provider device is configured to indicate if parameters of a) and/or b) are outside the second and/or the third sets of thresholds based on an output from the pressure analyzer unit, and thereby define a presence of instability of baseline pressure of the pressure sensor.

Another feature of the disclosure includes an apparatus in a pressure analyzing system to assess intracranial pressure (ICP) in a human.

A feature of the disclosure includes a system for assessing arterial blood pressure (ABP) in a human, the system comprising:

a pressure sensor that is insertable into a blood-vessel compartment or in communication with fluid of the blood-vessel compartment, the pressure sensor being configured to measure ABP signals, which represent differences in pressure between atmospheric pressure and pressure inside the blood-vessel compartment; and

a pressure analyzer unit in communication with the pressure sensor, the pressure analyzer unit being configured to:
process and analyze the ABP signals from the pressure sensor;
based on the processing and analyzing of the ABP signals, provide one or more baseline pressure indicator (BPi) plots created from pressure stability levels (SW.x.PSL) of definable time durations (SW.x.PSL.TD), calculated from single pressure wave (SW.x)-related parameters from a predefined number of single pressure waves having delta single pressure wave (dSW.x)-related parameters within a first set of thresholds, the first set of thresholds referring to defined pressure ranges of any one of parameters dSW.meanP and dSW.dP, and with beginning pressure differences and ending pressure differences for each pressure stability level (SW.x.PSL.PD), the beginning pressure difference being defined as a difference between a present pressure stability level and a previous pressure stability level, and the ending pressure difference being defined as the difference between a present pressure stability level and a next pressure stability level,

wherein the pressure analyzer unit has an outlet and an information provider device configured to provide information about the stability of baseline pressure of the pressure sensor from the BPi plot, the information being a function of at least one of:

a) combinations of pressure differences between different of the pressure stability levels (SW.x.PSL), calculated from a same type of single pressure wave (SW.x)-related parameters, the pressure differences being outside or inside a second set of thresholds, reflecting deviations from nominal reference pressure differences, and

b) relationships between different and simultaneous pressure stability levels (n−1; n) calculated from different types of single pressure wave (SW.x)-related parameters, the relationships being outside or inside a third set of thresholds, reflecting deviations from nominal reference relationships, wherein parameters of a) and/or b) outside the second set and/or the third set of thresholds define instability of baseline pressure of the pressure sensor, and

wherein the information provider device is configured to indicate if parameters of a) and/or b) are outside the second set and/or the third set of thresholds based on an output from the pressure analyzer unit, and thereby define a presence of instability of baseline pressure of the pressure sensor.

Another feature of the disclosure includes an apparatus in a pressure analyzing system to assess arterial blood pressure (ABP) in a human.

A feature of the disclosure includes a pressure analyzing system to assess cerebral perfusion pressure (CPP) in a human, i.e. mean arterial blood pressure (ABP) minus mean intracranial pressure (ICP), the system comprising:

a first pressure sensor that is insertable into a blood-vessel compartment or in communication with fluid of the blood-vessel compartment, the pressure sensor being configured to measure arterial blood pressure (ABP) signals, which represent differences in pressure between atmospheric pressure and pressure inside the blood-vessel compartment;

a second pressure sensor that is insertable into a cranio-spinal cavity or in communication with fluid of the cranio-spinal cavity, the pressure sensor being configured to measure intracranial pressure (ICP) signals, which represent differences in pressure between the atmospheric pressure and pressure inside the cranio-spinal cavity; and

a pressure analyzer unit in communication with the first and second pressure sensors and configured to process and analyze the ABP and ICP signals from the first and second pressure sensors to provide baseline pressure indicator (BPi) plots from the ABP and ICP signals, the BPi plots being created from pressure stability levels (SW.x.PSL) of definable time durations (SW.x.PSL.TD), calculated from single pressure wave (SW.x)-related parameters from a predefined number of single pressure waves having delta single pressure wave (dSW.x)-related parameters within a first set of thresholds, the first set of thresholds referring to defined pressure ranges of any one of parameters dSW.meanP and dSW.dP, and with beginning pressure differences and ending pressure differences for each pressure stability levels (SW.x.PSL.PD), the beginning pressure difference being defined as a difference between a present pressure stability level and a previous pressure stability level and the ending pressure difference being defined as a difference between a present pressure stability level and a next pressure stability level,

wherein the pressure analyzer unit has an outlet and an information provider device configured to provide information about the stability of baseline pressure of the pressure sensor from the BPi plots, the information being a function of at least one of:

a) combinations of pressure differences between different pressure stability levels (SW.x.PSL), calculated from a same type of single pressure wave (SW.x)-related parameters, the pressure differences being outside or inside a second set of thresholds, reflecting deviations from nominal reference pressure differences, and

b) relationships between different and simultaneous pressure stability levels (n−1; n) calculated from different types of single pressure wave (SW.x)-related parameters, the relationships being outside or inside a third set of thresholds, reflecting deviations from nominal reference relationships,

wherein parameters of a) and/or b) outside the second set and/or the third set of thresholds define instability of baseline pressure of the pressure sensor, and

wherein the information provider device is configured to indicate if parameters of a) and/or b) are outside the second set and/or the third set of thresholds based on an output from the pressure analyzer unit, and thereby define a presence of instability of baseline pressure of the pressure sensors.

A feature of the disclosure also includes an apparatus in a pressure analyzing system to assess cerebral perfusion pressure (CPP) in a human i.e. mean arterial blood pressure (ABP) minus mean intracranial pressure (ICP).

In a second aspect of the disclosure, means for correcting mean pressure that has been altered due to baseline pressure instability are described. The mean pressure, such as mean ICP and mean ABP, is used in the surveillance of patients. Since the baseline pressure instability may alter the mean pressure in wrong direction, means for correction of erroneous mean pressure may be useful. The second aspect of the disclosure includes novel methodology that may be incorporated in software.

A feature of the disclosure includes a method for correcting mean pressure alterations caused by instability of baseline pressure of a pressure sensor applied for sampling of continuous pressure signals originating from inside a human body or body cavity, samples of the pressure signals from the sensor being obtainable at specific intervals, and being convertible into pressure-related digital data with a time reference,

the method comprising:

from the digital data identification of single pressure waves related to cardiac beat-induced pressure waves,

detection of single pressure wave (SW.x)-related parameters, selectable from one or more of mean pressure (SW.meanP) and amplitude (SW.dP), and

based on the detection, computation of one or more delta single pressure wave (dSW.x)-related parameters between a selectable number of single pressure waves (n−1;n), representing differences in single pressure wave (dSW.x)-related parameters, selectable from one or more of change in mean pressure (dSW.meanP) and change in amplitude (dSW.dP) between a consecutive number of single pressure waves (n−1;n),

wherein pressure stability levels (SW.x.PSL) are created, each pressure stability level being created from consecutive single pressure waves (SW.x) having any one of delta single pressure wave (dSW.x)-related parameters dSW.meanP and dSW.dP within a first type of selectable thresholds, the first type of thresholds referring to defined pressure ranges of any one of the parameters dSW.meanP and dSW.dP, and wherein a pressure stability level refers to average of any one of the single pressure wave (SW.x)-related parameters SW.meanP and SW.dP,

wherein pressure differences between different of the pressure stability levels (n−1; n) (SW.x.PSL.PD) are determined, each of the pressure stability levels having definable time durations (SW.x.PSL.TD), relating to the time duration of the pressure stability levels (SW.x.PSL), incorporating a definable number of single pressure waves,

wherein the pressure stability levels (SW.x.PSL) of definable time durations (SW.x.PSL.TD) and with beginning and ending pressure differences (SW.x.PSL.PD) for each pressure stability level (SW.x.PSL) together creating a baseline pressure indicator (BPi) plot, the beginning pressure difference being defined as the difference between a present and a previous pressure stability level and the ending pressure difference being defined as the difference between a present and a next pressure stability level,

wherein information about stability of baseline pressure of the pressure sensor being a function of at least one of:

a) combinations of pressure differences between different of the pressure stability levels (SW.x.PSL), calculated from the same type of single pressure wave (SW.x)-related parameters, the pressure differences being outside or inside a second type of selectable set thresholds, reflecting deviations from nominal reference pressure differences, and

b) relationships between different and simultaneous pressure stability levels (n−1; n) calculated form different types of single pressure wave (SW.x)-related parameters, the relationships being outside or inside a third type of selectable set thresholds, reflecting deviations from nominal reference relationships, and

wherein parameters of a) and/or b) outside the respective thresholds define instability of baseline pressure of the pressure sensor, and

wherein levels of the mean pressure related to baseline pressure instability are corrected as a function of the pressure difference between pressure stability levels (SW.x.PSL.PD), the corrections being selectable according to defined criteria, and

wherein the corrected mean pressures are presented.

A feature of the disclosure includes a system for correcting mean pressure alterations caused by instability of baseline pressure of a pressure sensor applied for sampling of pressure signals originating from locations inside a human body or body cavity, the system comprising:

a transfer means configured to transfer the pressure signals from the pressure sensor to a sampling unit;

a signal converter in communication with the sampling unit and configured to perform conversion of sampled pressure signals into pressure-related digital data with a time reference;

an identifier unit to receive the pressure-related digital data from the signal converter and identify therefrom single pressure waves related to cardiac beat-induced pressure waves;

a detector coupled to an output of the identifier unit and configured to detect single pressure wave (SW.x)-related parameters, being one or more of:

single wave mean pressure (SW.meanP), and

single wave amplitude (SW.dP); and

a computing device coupled to an output of the detector and configured to compute one or more delta single pressure wave (dSW.x)-related parameters, representing differences in single pressure wave (dSW.x)-related parameters being one or more of change in mean pressure (dSW.meanP) and change in amplitude (dSW.dP) between a consecutive number of single pressure waves (n−1;n),

wherein a calculation unit is coupled to the computing device and configured to calculate pressure stability levels (SW.x.PSL), each pressure stability level being created from consecutive single pressure waves having any one of the delta single pressure wave (dSW.x)-related parameters dSW.meanP and dSW.dP within a first set of thresholds, the first set of thresholds referring to defined pressure ranges of any one of the parameters dSW.meanP and dSW.dP, wherein each pressure stability level refers to an average of any one of the single pressure wave (SW.x)-related parameters SW.meanP and SW.dP,

wherein a determination unit is coupled to the calculation unit and configured to determine pressure differences between different pressure stability levels (n−1; n) (SW.x.PSL.PD),

wherein the pressure stability levels (SW.x.PSL) of definable time durations (SW.x.PSL.TD) relating to a time duration of the pressure stability levels (SW.x.PSL) and with beginning pressure difference and ending pressure differences (SW.x.PSL.PD) for each pressure stability level (SW.x.PSL), together creating a baseline pressure indicator (BPi) plot, the beginning pressure difference being defined as the difference between a present pressure stability level and a previous pressure stability level and the ending pressure difference being defined as a difference between a present pressure stability level and a next pressure stability level,

wherein information about stability of baseline pressure of the pressure sensor is a function of at least one of:

a) combinations of the pressure differences between different pressure stability levels (SW.x.PSL), calculated from a same type of single pressure wave (SW.x)-related parameters, the pressure differences being outside or inside a second set of thresholds, reflecting deviations from nominal reference pressure differences, and

b) relationships between different and simultaneous pressure stability levels (n−1; n) calculated from different types of single pressure wave (SW.x)-related parameters, the relationships being outside or inside a third set of thresholds, reflecting deviations from nominal reference relationships, and

wherein parameters of a) and/or b) outside the second set and/or the third set of thresholds define a baseline pressure instability of the pressure sensor, and

wherein a mean pressure correcting unit is coupled to the determination unit and configured to correct the mean pressure (SW.meanP) levels related to the baseline pressure instability as a function of the pressure differences between different pressure stability levels (SW.x.PSL.PD), the corrections being selectable according to predefined criteria, and

wherein a presentation means is coupled to the mean pressure correcting unit and configured to present the corrected mean pressure.

In a third aspect of the disclosure, means for detection of baseline pressure instability and pressure correlation between ICP and ABP measurements are described. Determination of correlation between mean ICP and mean ABP is used in the surveillance of patients with brain damage. The baseline pressure instability may erroneously alter correlation indices between mean ICP and ABP measurements.

A feature of the disclosure includes a method for assessing information about stability of baseline pressure and pressure correlation of at least one intracranial pressure (ICP) sensor applied for sampling of continuous ICP signals originating from inside a cranio-spinal cavity and at least one arterial blood pressure (ABP) sensor applied for sampling of continuous ABP signals originating from inside a blood-vessel compartment, samples of the ICP and ABP signals from the ICP and ABP sensors being obtainable at specific intervals, and being convertible into pressure-related digital data with a time reference,

the method comprising:

identifying from the digital data of the ICP and ABP sensors single ICP and ABP waves related to cardiac beat-induced pressure waves,

and for each of the ICP and ABP signals:

detection from the digital data of single pressure wave (SW.x)-related parameters selectable from one or more of mean pressure (SW.meanP) and amplitude (SW.dP),

in a first mode:

based on the detection, computation of delta single pressure wave (dSW.x)-related parameters between a selectable number of single pressure waves (n−1;n), representing differences in single pressure wave (dSW.x)-related parameters selectable from one or more of change in mean pressure (dSW.meanP) and change in amplitude (dSW.dP) between a selectable number of single pressure waves (n−1;n),

and

in a second mode:

from the digital data, computation of correlation between one or more of the single pressure wave (SW.x)-related parameters selected from one or more of mean pressure (SW.meanP) and amplitude (SW.dP) of the ICP and ABP sensors, and

determination of magnitude of correlation between single pressure wave parameters of the ICP and ABP sensors,

wherein further in the first mode:

calculation of pressure stability levels (SW.x.PSL) each pressure stability level being created from consecutive single pressure waves having any one of delta single pressure wave (dSW.x)-related parameters dSW.meanP and dSW.dP within a first type of selectable thresholds, the thresholds referring to defined pressure ranges of any one of the parameters dSW.meanP and dSW.dP, and wherein a pressure stability level refers to average of any one of the single pressure wave (SW.x)-related parameters SW.meanP and SW.dP, and

determination of pressure differences between different of the pressure stability levels (n−1;n) (SW.x.PSL.PD),

and

creation of a baseline pressure indicator (BPi) plot using the pressure stability levels (SW.x.PSL) of definable time durations (SW.x.PSL.TD) relating to the time duration of the pressure stability levels (SW.x.PSL) and with beginning and ending pressure differences (SW.x.PSL.PD) for each pressure stability level (SW.x.PSL), the beginning pressure difference being defined as the difference between a present and a previous pressure stability level and the ending pressure difference being defined as the difference between a present and a next pressure stability level,

the plots providing information about stability of baseline pressure of the pressure sensor and being a function of at least one of:

a) combinations of pressure differences between different of the pressure stability levels (SW.x.PSL), calculated from the same type of single pressure wave (SW.x)-related parameters, the pressure differences being outside or inside a second type of selectable set thresholds, reflecting deviations from nominal reference pressure differences, and

b) relationships between different and simultaneous pressure stability levels (n−1; n) calculated from different types of single pressure wave (SW.x)-related parameters, the relationships being outside or inside a third type of selectable set thresholds, reflecting deviations from nominal reference relationships, and

wherein parameters of a) and/or b) outside the thresholds define instability of baseline pressure of the pressure sensor,

wherein further in the second mode:

presentation of information about magnitude of correlation between single ICP and ABP wave (SW.x) related parameters,

and

wherein an output is given to indicate whether the information in the second mode about magnitude of correlation between single ICP and ABP wave related parameters is accompanied with baseline pressure instability as defined in of the first mode.

A feature of the disclosure includes a system for assessing information about stability of baseline pressure and pressure correlation of at least one intracranial pressure (ICP) sensor applied for sampling of continuous ICP signals originating from inside a cranio-spinal cavity and at least one arterial blood pressure (ABP) sensor applied for sampling of continuous ABP signals originating from inside a blood-vessel compartment,

samples of the ICP and ABP signals from the ICP and ABP sensors being obtainable at specific intervals, and being convertible into pressure-related digital data with a time reference,

the system comprising:
transfer means being coupled to the ICP and ABP sensors and being configured to transferring the respective ICP and ABP signals to a sampling unit,
a signal converter in communication with the sampling unit and configured to perform conversion of sampled ICP and ABP signals into pressure-related digital data with a time reference,
an identifier unit to receive the digital data from the signal converter and identify therefrom ICP and ABP single pressure waves related to cardiac beat-induced pressure waves,
a detector being coupled to the identifier unit and being configured to detect from the respective ICP and ABP single pressure waves, single pressure wave (SW.x)-related parameters, being one or more of mean pressure (SW.meanP) and amplitude (SW.dP),
a first computing device coupled to the detector and configured for determination of stability of baseline pressure and configured to compute from the detected parameters of ICP and ABP single pressure waves, delta single pressure wave (dSW.x)-related parameters between a selectable number of single pressure waves (n−1;n), representing differences in single pressure wave (SW.x)-related parameters, being one or more of change in mean pressure (dSW.meanP) and change in amplitude (dSW.dP) between a consecutive number of single pressure waves (n−1;n),
a second computing device coupled to the detector and configured for computation of correlation and magnitude between one or more of the single pressure wave (SW.x)-related parameters being one or more of: mean pressure (SW.meanP) and amplitude (SW.dP) of the ICP and ABP sensors,

wherein the first computing device further being configured to:
in a calculation stage calculate pressure stability levels (SW.x.PSL), each pressure stability level being created from consecutive single pressure waves having any one of delta single pressure wave (dSW.x)-related parameters dSW.meanP and dSW.dP within a first type of selectable thresholds, the first type of thresholds referring to defined pressure ranges of any one of the parameters dSW.meanP and dSW.dP, and wherein a pressure stability level refers to average of any one of the single pressure wave (SW.x)-related parameters SW.meanP and SW.dP, and
in a determination stage to determine pressure differences between different of the pressure stability levels (n−1; n) (SW.x.PSL.PD),
a presentation unit configured to create baseline pressure indicator (BPi) plots from pressure stability levels (SW.x.PSL) of definable time durations (SW.x.PSL.TD) and with beginning and ending pressure differences (SW.x.PSL.PD) for each pressure stability level (SW.x.PSL),

wherein the plots providing information about stability of baseline pressure of the pressure sensor and being a function of at least one of:

a) combinations of pressure differences between different of the pressure stability levels (SW.x.PSL), calculated from the same type of single pressure wave (SW.x)-related parameters, the pressure differences being outside or inside a second type of selectable set thresholds, reflecting deviations from nominal reference pressure differences, and

b) relationships between different and simultaneous pressure stability levels (n−1; n) calculated from different types of single pressure wave (SW.x)-related parameters, the relationships being outside or inside a third type of selectable set thresholds, reflecting deviations from nominal reference relationships, and

wherein the presentation unit in a first stage parameters of a) and/or b) outside the threshold define pressure sensor instability and related baseline pressure instability,

wherein an output from the second computing device is connected to a second stage of the presentation unit, the second stage configured to provide presentation of information about magnitude of correlation between single ICP and ABP wave (SW.x) related parameters,

and

wherein the presentation unit has a third stage connected to output from the first stage and second stage, the third stage being configured for providing an output whether information from second stage is accompanied with pressure instability as defined from first stage.

In a fourth aspect of the disclosure, means for assessing baseline pressure instability and correction of mean pressure utilizing remote processing are described.

A features of the disclosure includes a system for assessing stability of baseline pressure of a pressure sensor being in communication with a pressure transducer system and capable of measuring continuous pressure signals from inside a human body or body cavity, wherein the system comprises:

an extension unit;

a remote processing unit; and

an output unit,

wherein the extension unit comprises:
a first transfer unit configured to transfer continuous pressure signals from the pressure sensor to a sampling unit,
a signal converter in communication with the sampling unit and configured to perform conversion of sampled continuous pressure signals into pressure-related digital data with a time reference,
a decryption unit, configured to de-identify the pressure-related digital data for sensitive information, resulting in de-identified pressure-related digital data, and
a second transfer unit configured to transmit the de-identified pressure-related digital data to the remote processing unit,

wherein the remote processing unit comprises:
an analyzer unit configured to analyze the de-identified pressure-related digital data, and
a third transfer unit configured to transmit an analysis output from the analyzer unit to the output unit, wherein the output unit is configured to provide an output of the remote processing unit, the output presenting baseline pressure instability of a pressure sensor.

Furthermore, the analyzer unit of the remote processing unit comprises an identifier unit configured to receive the de-identified pressure-related digital data from the second transfer unit and identify single pressure waves related to cardiac beat-induced pressure waves from the de-identified pressure-related digital data,

a detector connected to an output of the identifier unit and configured to detect single pressure wave (SW.x)-related parameters from the single pressure waves, being at least one or more of mean pressure (SW.meanP) and amplitude (SW.dP); and

a computing device connected to an output of the detector and configured to compute one or more of delta single pressure wave (dSW.x)-related parameters representing differences in single pressure wave (dSW.x)-related parameters being one or more of change in mean pressure (dSW.meanP), and change in amplitude (dSW.dP), between a consecutive number (n−1;n) of single pressure waves (SW.x),

wherein a calculation unit is connected to an output of the computing device and configured to calculate pressure stability levels (SW.x.PSL), each pressure stability level being created from consecutive single pressure waves having any one of the delta single pressure wave (dSW.x)-related parameters dSW.meanP and dSW.dP within a first set of thresholds, the first set of thresholds referring to defined pressure ranges of any one of the parameters dSW.meanP and dSW.dP, wherein each pressure stability level (SW.x.PSL) refers to an average of any one of the single pressure wave (SW.x)-related parameters SW.meanP and SW.dP,

wherein a determination unit is connected to an output of the calculation unit and configured to determine pressure differences (SW.x.PSL.PD) between different pressure stability levels (n−1;n) (SW.x.PSL),

wherein the pressure stability levels (SW.x.PSL) have definable time durations (SW.x.PSL.TD) relating to a time duration of the pressure stability levels (SW.x.PSL),

wherein a presentation unit is connected to an output of the determination unit and configured to present baseline pressure indicator (BPi) plots, being created from pressure stability levels (SW.x.PSL) and with beginning pressure differences and ending pressure differences (SW.x.PSL.PD) for each pressure stability level (SW.x.PSL), the beginning pressure difference being defined as a difference between a present pressure stability level and a previous pressure stability level and the ending pressure difference being defined as a difference between a present pressure stability level and a next pressure stability level,

wherein the BPi plots provide information about stability of baseline pressure of the pressure sensor and are a function of at least one of:

i) combinations of the pressure differences between different pressure stability levels (n−1; n) (SW.x.PSL), calculated from a same type of single pressure wave (SW.x)-related parameters, the pressure differences being outside or inside a second set of thresholds, reflecting deviations from nominal reference pressure differences, and

ii) relationships between different and simultaneous pressure stability levels (n−1; n) (SW.x.PSL) calculated from different types of single pressure wave (SW.x)-related parameters, the relationships being outside or inside a third set of thresholds, reflecting deviations from nominal reference relationships, and

wherein the presentation unit is configured to indicate if parameters of i) and/or ii) are outside the second set and/or the third set of thresholds and thereby define instability of baseline pressure of the pressure sensor.

Moreover, the system may incorporate a mean pressure correction unit connected to an output of the analyzer unit of the remote processing system and being configured to correct mean pressure (SW.meanP) levels related to baseline pressure instability as a function of the pressure differences between different of the pressure stability levels (SW.x.PSL.PD), the correction of mean pressure being selectable according to defined criteria, and wherein the correction unit provides for a presentation of corrected mean pressure.

A feature of the disclosure includes a method for assessing stability of baseline pressure of a pressure sensor in communication with a pressure transducer system, the pressure sensor being capable of measuring continuous pressure signals from inside a human body or body cavity,

the method comprising:

receiving, from the pressure sensor, continuous pressure signals measured from inside a human body or a body cavity;

sampling, by one or more computing devices, the continuous pressure signals to sampled continuous pressure signals;

converting, by the one or more computing devices, the sampled continuous pressure signals into pressure-related digital data with a time reference;

de-identifying, by the one or more computing devices, the pressure-related digital data for sensitive information, resulting in de-identified pressure-related digital data;

transmitting, by the one or more computing devices, the de-identified pressure-related digital data for remote processing, the remote processing comprising an analysis of the de-identified pressure-related digital data and an identification of a baseline pressure instability of the pressure sensor;

transmitting, by the one or more computing devices, information about the baseline pressure instability; and

providing, by the one or more computing devices, an output of the remote processing, the output presenting the baseline pressure instability of the pressure sensor.

Furthermore, the analysis of remote processing comprises:

identifying, from the de-identified pressure-related digital data, single pressure waves related to cardiac beat-induced pressure waves,

detecting, from the single pressure waves, single pressure wave (SW.x)-related parameters, selectable from one or more of mean pressure (SW.meanP) and amplitude (SW.dP),

computing one or more of delta single pressure wave (dSW.x)-related parameters, representing differences in single pressure wave (dSW.x)-related parameters and comprising one or more of a change in mean pressure (dSW.meanP), and a change in pressure amplitude (dSW.dP), between a consecutive number of single pressure waves (n−1;n),

calculating pressure stability levels (SW.x.PSL), each pressure stability level being created from consecutive single pressure waves having any one of the delta single pressure wave (dSW.x)-related parameters dSW.meanP and dSW.dP within a first set of thresholds, the first set of thresholds referring to defined pressure ranges of any one of the parameters dSW.meanP and dSW.dP, and wherein each pressure stability level refers to an average of any one of the single pressure wave (SW.x)-related parameters SW.meanP and SW.dP,

determining pressure differences (SW.x.PSL.PD) between different pressure stability levels (n−1;n) (SW.x.PSL), wherein the pressure stability levels (SW.x.PSL) have definable time durations (SW.x.PSL.TD) relating to a time duration of the pressure stability levels (SW.x.PSL), and

presenting baseline pressure indicator (BPi) plots being created from pressure stability levels (SW.x.PSL) and with beginning pressure difference and ending pressure differences (SW.x.PSL.PD) for each pressure stability level (SW.x.PSL), the beginning pressure difference being defined as the difference between a present pressure stability level and a previous pressure stability level, and the ending pressure difference being defined as the difference between a present pressure stability level and a next pressure stability level,

wherein the BPi plots provide information about the stability of baseline pressure of the pressure sensor and are a function of at least one of:

i) combinations of the pressure differences between different pressure stability levels (SW.x.PSL), calculated from a same type of single pressure wave (SW.x)-related parameters, the pressure differences being outside or inside a second set of thresholds, reflecting deviations from nominal reference pressure differences, and

ii) relationships between different and simultaneous pressure stability levels (n−1; n) (SW.x.PSL) calculated from different types of single pressure wave (SW.x)-related parameters, the relationships being outside or inside a third set of thresholds, reflecting deviations from nominal reference relationships, and

wherein the presenting of the BPi plots comprises indicating if parameters of i) and/or ii) are outside the second set and/or the third set of thresholds and thereby defining the baseline pressure instability of the pressure sensor.

The method may, in a correction step, enable the single wave mean pressure (SW.meanP) to undergo correction as a function of the pressure differences between the different pressure stability levels (SW.x.PSL.PD), the correction being selectable according to defined criteria, and wherein corrected mean pressures based on the correction are presented.

Further features and advantages, as well as the structure and operation of various embodiments, are described in detail below with reference to the accompanying drawings. It is noted that the specific embodiments described herein are not intended to be limiting. Such embodiments are presented herein for illustrative purposes only. Additional embodiments will be apparent to persons skilled in the relevant art(s) based on the teachings contained herein.

### BRIEF DESCRIPTION OF THE DRAWINGS

FIG. 1 illustrates Table 2, which shows a distribution of SW.MeanP/SW.dP ratios, according to embodiments of the present disclosure.

FIG. 2 illustrates Table 3, which shows a distribution of SW.MeanP/SW.dP ratios for different levels of SW.MeanP, according to embodiments of the present disclosure.

FIG. 3 illustrates Table 4, which shows a distribution of SW.MeanP/SW.dP ratios for different levels of SW.MeanP. SW.MeanP/SW.dP ratios, according to embodiments of the present disclosure.

FIG. 4 illustrates Table 5, which shows a distribution of dSW.MeanP/dSW.dP ratios, according to embodiments of the present disclosure.

FIG. 5 illustrates Table 6, which shows a distribution of dSW.MeanP/dSW.dP ratios depending on dSW.MeanP levels, according to embodiments of the present disclosure.

FIG. 6 illustrates Table 7, which shows a distribution of dSW.MeanP/dSW.dP ratios depending on dSW.dP levels, according to embodiments of the present disclosure.

FIG. 7a shows trend plots of mean ICP (ICP.SW.MeanP) and mean ICP wave amplitude (ICP.SW.dP) measured from a Camino ICP sensor placed in the right frontal horn. FIG. 7b shows trend plots of mean ICP (ICP.SW.MeanP) and mean ICP wave amplitude (ICP.SW.dP) measured simultaneously from a Codman ICP sensor placed in the right frontal horn nearby the Camino ICP sensor.

FIG. 8a shows single intracranial pressure waves (SWs) plotted over time, and with a trend plot of mean pressure (ICP.SW.meanP) presented as a line. FIGS. 8b-c illustrate the determination of delta single wave (dSW.x) parameters between two subsequent single pressure waves (SWₙ versus SWₙ₋₁). FIG. 8d shows the plotting of a dSW.meanP/dSW.dP ratio over time for one continuous ICP signal. The vertical spikes demonstrate dSW.meanP/dSW.dP ratios of high magnitudes.

FIGS. 9a-d show trend plots of mean intracranial pressure (ICP.SW.meanP) and amplitude (ICP.SW.dP) and pressure stability levels of the respective single wave parameters (ICP.SW.meanP.PSL; ICP.SW.dP.PSL). It is illustrated how changing selectable first type of thresholds impact the calculation of pressure stability levels (SW.x.PSL).

FIGS. 10a-d show trend plots of mean intracranial pressure (ICP. SW.meanP) and amplitude (ICP.SW.dP) and pressure stability levels for mean pressure (ICP.SW.meanP.PSL) and amplitude (ICP.SW.dP.PSL). It is illustrated how changing selectable first type of thresholds impact the calculation of pressure stability levels (SW.x.PSL).

FIGS. 11a-c provide schematic illustrations of different types of pressure stability levels (SW.x.PSL), and show different combinations of pressure stability levels for mean pressure (ICP.SW.meanP.PSL) and amplitude (ICP.SW.dP.PSL).

FIGS. 12a-c show the creation of a baseline pressure indicator (BPi) plot for mean intracranial pressure (ICP. SW.meanP) from creation of pressure stability levels (ICP.SW.meanP.PSL) and pressure difference between pressure stability levels (ICP.SW.meanP.PSL.PD).

FIGS. 13a-c show the creation of a baseline pressure indicator (BPi) plot for mean intracranial pressure (ICP.SW.meanP) from pressure stability levels (ICP.SW.meanP.PSL) and pressure difference between pressure stability levels (ICP.SW.meanP.PSL.PD).

FIGS. 14a-c show the creation of a baseline pressure indicator (BPi) plot for mean intracranial pressure (ICP.SW.meanP) from pressure stability levels (ICP.SW.meanP.PSL) and pressure difference between pressure stability levels (ICP.SW.meanP.PSL.PD).

FIG. 15 illustrates Table 10, which shows combinations of ICP.SW.meanP.PSL.PD and ICP.SW.meanP.PSL.TD from a cohort of 601 observations, according to embodiments of the present disclosure.

FIGS. 16a-g show different examples of baseline pressure indicator (BPi) plots. FIGS. 16a-b show trend plots of mean ICP (ICP. SW.MeanP) and mean ICP wave amplitude (ICP.SW.dP) and the corresponding BPi plots from simultaneous ICP measurements from two different ICP sensors. FIG. 16c-d present only the BPi plots. FIGS. 16e-f shows trend plots of mean ICP (ICP.SW.MeanP) and mean ICP wave amplitude (ICP.SW.dP) and corresponding BPi plots from one single ICP measurement. FIG. 16g shows another example of measurement from one single ICP sensor, showing trend plots of mean intracranial pressure (ICP.SW.meanP) and amplitude (ICP.SW.dP), and the baseline pressure indicator (BPi) plots for mean pressure (ICP.SW.meanP) superimposed on the trend plot of mean pressure (ICP.SW.meanP), and the baseline pressure indicator (BPi) plot for amplitude (ICP.SW.dP) superimposed on the trend plot of amplitude (ICP.SW.dP).

FIG. 17 illustrates a method for assessing baseline pressure instability of a pressure sensor applied for sampling of continuous pressure signals, according to embodiments of the present disclosure.

FIG. 18 illustrates a system for assessing stability of baseline pressure of a pressure sensor applied for sampling of continuous pressure signals, according to embodiments of the present disclosure.

FIG. 19 illustrates a pressure analyzing system configured to assess ICP, according to embodiments of the present disclosure.

FIG. 20 illustrates an apparatus comprising a pressure sensor and a pressure analyzer unit communicating with the pressure sensor, configured to assess ICP, according to embodiments of the present disclosure.

FIG. 21 illustrates a pressure analyzing system configured to assess ABP, according to embodiments of the present disclosure.

FIG. 22 illustrates an apparatus comprising a pressure sensor in communication with a pressure analyzer unit, configured to assess ABP, according to embodiments of the present disclosure.

FIG. 23 illustrates a pressure analyzing system configured to assess CPP, according to embodiments of the present disclosure.

FIG. 24 illustrates an apparatus in a pressure analyzing system to assess cerebral perfusion pressure (CPP) in a human, according to embodiments of the present disclosure.

FIG. 25 illustrates a method for correcting mean pressure alterations caused by instability of baseline pressure of a pressure sensor applied for sampling of continuous pressure signals, according to embodiments of the present disclosure.

FIG. 26 illustrates a system for correcting mean pressure alterations caused by instability of baseline pressure of a pressure sensor applied for sampling of continuous pressure signals, according to embodiments of the present disclosure.

FIGS. 27a-b illustrate the correction of mean intracranial pressure (ICP.SW.meanP) from the baseline pressure indicator (BPi) plot, according to embodiments of the present disclosure. FIG. 27a shows the trend plot of non-corrected mean intracranial pressure (ICP.SW.meanP), while FIG. 27b shows the trend plot of corrected mean intracranial pressure (ICP.SW.meanP_{CORR}) in addition to the trend plot of non-corrected mean intracranial pressure (ICP.SW.meanP).

FIGS. 28a-b illustrate the combined plotting over time of (FIG. 28a) baseline pressure indicator (BPi) plot and (FIG. 28b) pressure correlation index, according to embodiments of the present disclosure.

FIG. 29 illustrates a method for assessing information about stability of baseline pressure and pressure correlation of at least one intracranial pressure (ICP) sensor applied for sampling of continuous ICP signals originating from inside a cranio-spinal cavity and at least one arterial blood pressure (ABP) sensor applied for sampling of continuous ABP signals originating from inside a blood-vessel compartment, according to embodiments of the present disclosure.

FIG. 30 illustrates a system for assessing information about stability of baseline pressure and pressure correlation of at least one intracranial pressure (ICP) sensor applied for sampling of continuous ICP signals originating from inside a cranio-spinal cavity and at least one arterial blood pressure (ABP) sensor applied for sampling of continuous ABP signals originating from inside a blood-vessel compartment, according to embodiments of the present disclosure.

FIG. 31 illustrates a system for assessing stability of baseline pressure of a pressure sensor applied for sampling of continuous pressure signals utilizing a remote processing system, according to embodiments of the present disclosure.

FIG. 32 illustrates a method for assessing baseline pressure instability of a pressure sensor applied for sampling of continuous pressure signals utilizing remote processing, according to embodiments of the present disclosure.

FIG. 33 illustrates a diagram of a pressure sensor and remote monitoring of data, according to embodiments of the present disclosure.

FIG. 34 illustrates a diagram of multiple pressures sensors connected to multiple systems and remote monitoring, according to embodiments of the present disclosure.

FIG. 35 illustrates a block diagram of example components of a computer system, according to embodiments of the present disclosure.

Embodiments of the present disclosure will be described with reference to the accompanying drawings.

### DETAILED DESCRIPTION

Although specific configurations and arrangements are discussed, it should be understood that this is done for illustrative purposes only. A person skilled in the pertinent art will recognize that other configurations and arrangements can be used without departing from the spirit and scope of the present disclosure. It will be apparent to a person skilled in the pertinent art that this disclosure can also be employed in a variety of other applications.

It is noted that references in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases do not necessarily refer to the same embodiment. Further, when a particular feature, structure or characteristic is described in connection with an embodiment, it would be within the knowledge of one skilled in the art to effect such feature, structure or characteristic in connection with other embodiments whether or not explicitly described.

The present disclosure provides systems, methods, and devices, for the assessment of baseline pressure instability of pressure sensors, and correction of mean pressure readings that have been altered by baseline pressure instability.

An overview of abbreviations used in this document is provided in Appendix A.

Invasive intracranial pressure (ICP) monitoring has an important role in the diagnosis and surveillance of patients with various types of brain damage or brain disease. For surveillance of patients with brain damage, e.g., due to trauma, stroke or as a complication to brain surgery, usually the ICP is measured together with arterial blood pressure (ABP). The so-called cerebral perfusion pressure (CPP) is computed according to this formula: mean CPP=mean ABP—mean ICP, and is an important parameter for patient surveillance. The common treatment goals are to keep ICP <20 mmHg and CPP >50-60 mmHg. This is done to avoid compromised blood flow to the brain, which is the main source of energy delivery to brain cells. Since the cranium is rigid without ability to expand (e.g., after about 2 years age), any disease process increasing the volume of intracranial components may cause increased ICP, which may hamper blood flow to the brain. In this context, monitoring of ICP and ABP is crucial.

A wide range of commercial pressure sensors are available, some of which are listed in Table 1. Even though the present disclosure primarily relates to measurements of ICP and ABP, these pressures do not represent a limitation of the present disclosure since assessment of baseline pressure instability is relevant whenever an absolute pressure is measured in humans.

TABLE 1
Examples of pressures and pressures sensors that may be used for
baseline pressure instability measurements.

| Sensor | | | |
|---|---|---|---|
| category | Pressure | Name of sensor | Manufacturer |
| Solid | ICP | Codman Microsensor ICP | Integra LifeSciences, Plainsboro |
| | | | NJ, USA |
| Solid | ICP | Raumedic Neuro Vent P | Raumedic AG, Münchberg, GE |
| Solid | ICP | Raumedic NeuroDur sensor | Raumedic AG, Münchberg, GE |
| | (Epidural) | | |
| Solid | ICP | Pressio ICP | Sophysa, Orsay, France |
| Solid | ICP | Camino ICP | Natus Medical Inc., WI, USA |
| Fiberoptic | ICP | Camino ICP | Natus Medical Inc., WI, USA |
| Fluid-based | ICP/ABP | Truwave pressure | Edwards Life sciences LLC, |
| | | transducers | Irvine, CA, USA |
| Fluid-based | ICP/ABP | B Braun single channel | B Braun AG, GE |
| | | invasive blood pressure | |
| | | transducer | |
| Fluid-based | ICP/ABP | Edwards Invasive blood | Edwards Life sciences LLC, |
| | | pressure transducer | Irvine, CA, USA |
| Air-pouch | ICP | Spiegelberg | Spiegelberg-Aesculap, GE |
| | | intraparenchymal probe 3PN | |

As indicated in Table 1, measurements of invasive ICP can be done using solid ICP sensors or by fluid-filled catheters. In the latter situation, the sensor element may be outside the body, and a catheter system enables contact between CSF and the pressure sensor. Similarly, invasive ABP monitoring implies that a catheter is placed within a blood vessel, and the ABP measured against a baseline pressure, which is the atmospheric pressure. Usually pressures in the human body is measured as millimeter mercury (mmHg) but may as well be measured as Pascal (Pa), or even centimeter of water (cm H₂O). In some embodiments, pressure measurements described herein are measured in mmHg, though this should not be construed as a limitation of the disclosure.

Human pressure measurements differentiate between static and pulsatile pressures. The static pressure is the absolute pressure difference against a baseline pressure. The pressure sensor is zeroed against the atmospheric pressure and pressure scores are, e.g., mean ICP and mean ABP. The baseline pressure may also be denoted reference pressure or set pressure. The pulsatile pressures, on the other hand, refer to the pressure changes occurring during the cardiac cycle. The notation pulse refers to the cardiac contractions, which is the input for the arterial pulse and the pulse pressure measured in other organs, e.g., the intracranial compartment. Assessment of the pulsatile ICP includes continuous sampling of pressure signals, such as at a frequency above 30-50 Hz, while assessment of static ICP may not.

Presently, ICP is most commonly measured by an invasive procedure, that is, the pressure sensor is placed within the scull during a surgical procedure. Solid pressure sensors are implanted within the cavity from which the pressure is measured, and pressure signals are wire-based transferred to a pressure transducer and conveyed to a monitor to display the measured pressures. The sensor may as well be permanently implanted into the cavity, for example within the epidural space, or it may be implanted together with a shunt drainage system for drainage of CSF and simultaneously measuring CSFP. Some commercial systems allow the pressure signals to be transferred by wire-less means to a pressure transducer, and to a monitor for display of the measured pressures. Furthermore, using miniature ICP sensor systems with transfer of pressure signals by wireless means, the ICP measurements may be performed for long periods of weeks and even months. The role of pressure sensor instability is even more important when measuring pressures with these systems.

The current practice of invasive monitoring of human pressures, e.g., ICP and ABP, is pressure measurements against a baseline (or reference) pressure value. Hence, the absolute pressure is being measured. The zero pressure at the start of a pressure measurement corresponds to the atmospheric pressure (0 mmHg). Some pressure monitoring systems allow for measuring the atmospheric zero pressure during a monitoring. Therefore, before a solid ICP sensor is implanted within a cranio-spinal cavity, it is zeroed against the atmospheric pressure. The ICP level displayed on the monitor represents the difference between pressure level within the intracranial compartment and the sensor zero point. The ICP being measured can be expressed according to equation (1):

P_{M}=P_{C}+P₀ Eq. (1)

The measured pressure (P_{M}) is the sum of the pressure within the cavity (P_{C}) and the baseline pressure (P₀). When the baseline pressure (P₀) is the atmospheric pressure, it is assumed that the atmospheric pressure is about zero mmHg. This practice is based on the concept that the baseline pressure (or reference pressure) of a pressure sensor is stable and not deviating extensively during ongoing pressure measurements. On the other hand, if the pressure sensor and the related baseline pressure are instable the ICP, ABP and CPP also become altered, not because of physiological changes but due to other causes such as pressure sensor instability. Accordingly, even though the atmospheric pressure does not change, inherent properties of the pressure sensor and measurement technology may affect the baseline pressure, causing the baseline pressure (P₀) to vary spontaneously during ongoing in vivo measurements, and resulting in baseline pressure instability (BPI). Previously, the prevalent idea among health care personnel is that baseline pressure instability of pressure sensors occurs very seldom. Therefore, this this topic has received minimal interest. However, baseline pressure instability is an important and frequent phenomena that heavily affects interpretation of pressure measurements and therefore calls for attention.

For the sake of clarity, the term baseline pressure (P₀) has also been denoted the set pressure, calibration pressure or reference pressure. Other terms may also be in use. There is presently no consensus on the preferred notation. In some embodiments, the term baseline pressure may be used herein, though this notation represents no limitation with the disclosure, and other terms such as the reference pressure might as well have been used.

To better explain formula (1), one theoretical example is given. If the cavity is an intracranial compartment such as the brain parenchyma, the ventricular fluid or the epidural space, the measured ICP (P_{M}) represents the sum of the pressure within the intracranial compartment (P_{C}) and the baseline pressure (P₀). If the measured mean ICP (=P_{M}) of an individual is 18 mmHg, a change in baseline pressure (=P₀) from 0 to 22 mmHg, will cause the measured or displayed mean ICP (=P_{M}) to reveal 40 mmHg (P_{M}=P_{C}+P₀=18 mmHg+22 mmHg=40 mmHg). Notably, the change in P_{REF} from 0 to 22 mmHg do not refer to a change in atmospheric pressure, but to alterations in P_{REF} of other reasons. It should also be noted that in addition to impacting mean ICP, changes in baseline pressure (=P₀) may erroneously affect mean ICP derived parameters such as cerebral perfusion pressure (CPP) and pressure-reactive index (PRx). If the end-user is not notified by change in P_{REF}, wrong actions may be taken to correct for changes in P_{M}.

There has been considerable focus on drift of baseline or reference pressure of pressure sensors used for e.g., ICP measurements. It is generally acknowledged that baseline pressure of pressure sensors may gradually change during monitoring over longer periods such as 1-4 weeks, though the pressure change is minor (<2-3 mmHg). However, this phenomenon is not examined in vivo, but with the pressure sensor placed in a fluid solution for several days. It has been focused on impact of temperature changes and changes in atmospheric pressure. Another method has been to check the baseline pressure of a pressure sensor after removal from a human. For example, the baseline pressure of an ICP sensor has been checked in atmospheric pressure after it has been removed from the intracranial cavity of the patient. Typically, pressure sensor drift is characterized by a change in baseline pressure, as compared to baseline pressure before insertion in the patient. The literature data suggest that the magnitude of drift of commercial ICP sensors is small, usually in the range 1-3 mmHg. For this reason, manufacturers of pressure sensors test the sensor's ability to drift in the laboratory; the specifications of the particular sensor usually details the pressure sensor's tendency to drift. However, despite these efforts from manufacturers, today's technologies do not provide the opportunity to measure temporary drift or shift in baseline pressure in vivo during ongoing pressure measurements. Notably, the present disclosure is not limited to drift of pressure sensors, but rather address pressure sensor instability causing sudden jumps and high-magnitude transient changes in baseline pressure.

The literature has addressed to a very limited degree to which extent and magnitude the baseline pressure (P₀) may vary during ongoing pressure measurements. Spontaneous changes in baseline pressure could be one explanation behind wrong ICP measurements, which occur rather frequent in the clinical setting. However, none of the currently used pressure monitoring systems incorporate means for detecting pressure sensor instability and related baseline pressure instability or providing issuance of alert if pressure sensors and related baseline pressure instability occurs. No methods have been established for determining baseline pressure instability from pressure sensors. Therefore, during pressure measurements users may not warned about the occurrence of baseline pressure instability, even though instability of baseline pressure (P₀) would impact the commonly used pressure scores, such as mean ICP, mean ABP, mean CPP, or the pressure-derived index PRx.

Given the minor interest in baseline pressure instability of pressure sensors, there is limited knowledge what causes spontaneous changes in baseline pressure. Possible causes are external factors such as electrostatic discharges, and sensor-specific causes including any of the technical components of a pressure sensor system (sensor, cable, transducer, display). Moreover, human factors may as well impact the baseline pressure erroneously. For example, wrong zeroing during implantation may be one cause of erroneous baseline pressure. Regarding ICP, the Codman and Camino ICP sensors are zeroed only prior to implantation, while no zeroing can be done after implantation. The Raumedic sensor has the ability for post-implantation electrical zeroing but has not the ability for a true in-vivo (atmospheric pressure) check of the catheter sensor. In addition, damage to the sensor during implantation or at any other point of time, may be caused by human factors and may result in pressure sensor instability and related baseline pressure instability. The same result may be caused by wrong positioning of the pressure sensor that may cause instability of the pressure sensor. The present disclosure is not limited to the possible cause(s) of baseline pressure instability, but addresses how to determine presence of baseline pressure instability of pressure sensors. Moreover, the disclosure addresses how erroneous pressure readings caused by baseline pressure instability may be corrected.

Taken together, while it is well established that pressure sensors may become instable for many reasons, current pressure measurement systems lack means to assess pressure sensor instability and related baseline pressure instability. There is an urgent need to develop means for assessing this issue as well as solutions to correct erroneous pressure readings. The present disclosure addresses these technical issues.

The following publications will be referred to herein, according to this list:
Reference 1: Eide P K, Bakken A, The baseline pressure of intracranial pressure (ICP) sensors can be altered by electrostatic discharges, BioMedical Engineering OnLine 2011, 10: 75.
Reference 2: Eide P K, Holm S, Sorteberg W, Simultaneous monitoring of static and dynamic intracranial pressure parameters from two separate sensors in patients with cerebral bleeds: Comparison of findings, BioMedical Engineering OnLine, 2012, 11: 66.
Reference 3: Eide P K, Sorteberg A, Meling T R, Sorteberg W, Baseline pressure (BPEs) extensively influence intracranial pressure scores: results of a prospective observational study, BioMedical Engineering OnLine 2014, 13: 7.
Reference 4: Eide P K, Sorteberg W, An intracranial pressure-derived index monitored simultaneously from two separate sensors in patients with cerebral bleeds: comparison of findings, Biomedical Engineering OnLine, 2013, 12: 14.
Reference 5: Eide P K, Sorteberg A, Meling T R, Sorteberg W, The effect of baseline pressure errors on an intracranial pressure-derived index: results of a prospective observational study, BioMedical Engineering OnLine, 2014, 13: 99.

In bench test experiments, it has been found that electrostatic discharges may alter the baseline pressure (or reference pressure) of pressure sensors configured to measure ICP (Reference 1). The Codman micro ICP sensors and Raumedic ICP sensors (Neurovent and NeuroDur) were highly sensitive to electrostatic discharges. In an experimental laboratory setup, a test person was charged, and an electrostatic discharge delivered to the pressure sensors. This caused both abrupt changes in baseline or reference pressure, with pressure changes in the range of 5-30 mmHg. Furthermore, electrostatic discharges caused major drifts in baseline pressure occurring over short time. Evidence was given that leakage current from pressure sensors caused drift of baseline pressure. These observations in the laboratory showed that inherent pressure sensor properties could permanently change the baseline pressure.

To examine in vivo whether baseline pressure from implantable ICP sensors changes during ongoing pressure measurements, pressure measurements performed simultaneously from two separate ICP sensors within a cranial compartment were examined. It was found that in some situations the static pressure (mean ICP) differed substantially even though the pulsatile ICP (mean ICP wave amplitude, MWA) was close to identical. The observations were evident for different types of ICP sensors, and were interpreted as erroneous alterations in baseline pressure that change the static ICP, while keeping the pulsatile ICP unaltered (Reference 2).

In order to explore how frequent baseline pressure errors occur, a prospective and observational study examined to which extent static ICP differs between two separate ICP sensors placed nearby within the brain. Substantial differences in mean ICP despite close to identical MWA measurements were observed in a proportion of pressure ICP recordings (Reference 3). In this latter study, marked differences in static ICP (mean ICP) without accompanying changes in pulsatile ICP (mean ICP wave amplitude, MWA) were referred to as baseline pressure errors (BPEs). This study defined BPEs as significantly different mean ICP between two ICP sensors despite close to identical ICP waveforms. Three different categories of baseline pressure errors were described:

i) Baseline pressure error caused by erroneous calibration of the pressure sensor typically results in lasting alterations in P_{REF}, which may be referred to as baseline pressure error (BPE) Type I.

ii) In another situation, baseline pressure instability may result in sudden change of P_{REF}, causing baseline pressure to stay at another level more or less permanently. This resulting error was denoted baseline pressure error Type II. The impact of Type II depends on both magnitude and duration of pressure change. A sudden change in P_{REF} is of a certain magnitude and lasts for some time to have impact on pressure measurements. iii) Finally, instability of baseline pressure may cause gradual deviation of P_{REF}; resulting in P_{REF} reaching another permanent level. This latter type of error was denoted baseline pressure error type III. While Type II might occur abrupt, e.g., during less than 1 second, the Type III was assumed to occur over several seconds, and even minutes. It is important to note that the previously described baseline pressure errors are deciphered from comparing measurements from two different ICP sensors. The study disclosed that pressure sensor instability and related baseline pressure instability are a common phenomenon during pressure monitoring.

In other studies, erroneous alterations in baseline pressure were shown to also affect ICP-derived indices (References 4 and 5). These studies showed that mean ICP-derived indices would also be affected by BPEs.

These reports suggested that transient changes in baseline or reference pressure occur during ingoing pressure measurements, but gave no technical solutions to the problems. It was proposed that BPEs might be determined by relating mean ICP and MWA during ongoing monitoring from one ICP sensor, namely that a sudden change in mean ICP not accompanied by a change in the ICP wave amplitude might provide an indication of the occurrence of a BPE.

Based on these reports, one strategy to measure baseline pressure instability would be to measure pressure from two separate pressure sensors. This would, however, neither be possible due to risk, nor feasible due to cost of pressure sensors. Another strategy might be to temporarily remove the sensor and check baseline pressure against atmospheric pressure. This can neither be done since removing and replacing sensor imposes risk and discomfort to the individuals undergoing pressure monitoring. A third strategy would be to relate the static and pulsatile pressures when both the static and pulsatile pressures are measured from one pressure sensor, based on a concept that baseline pressure errors result from major change in static pressure despite minor change in pulsatile pressure. This third alternative has, however, previously not been described, but might be considered one strategy. Therefore, a series of experiments were performed to explore how measurements of static and pulsatile pressure might be utilized to provide information about changes in baseline pressure. The aim was to establish an automatic procedure for identification of BPEs type 1 to 3. Experiments and testing were implemented. This exercise turned out to be difficult because biological measures such as static and pulsatile pressures are in constant change. In humans, this is related to body movement, respiration, blood pressure variations and physical activity. It may be impossible to know whether a change in the relationship between static and pulsatile ICP is related to physiological or technological effects. Particularly noise may cause short-lasting alterations in the relationship between static and pulsatile pressure, though not being representative of baseline pressure error.

In a first series of experiments, it was examined whether the relationship between levels on static pressure (mean ICP) and pulsatile amplitude pressure (mean ICP wave amplitude, MWA) could be indicative of occurrence of baseline pressure instability. However, these experiments showed that this relationship is highly variable. In fact, very different relationships may provide the same result. In another series of experiments, the relationship between difference in static mean pressure at single wave level (dSW.MeanP) was examined; it was assumed that difference in amplitude of single waves (dSW.dP) could be used to identify instability of baseline pressure. It was found that also the dSW.MeanP/dSW.dP relationship changes constantly during pressure measurements. A wide distribution in changes of each parameter could provide similar output. Accordingly, a search for certain combinations of dSW.MeanP/dSW.dP being indicative of baseline pressure errors was not successful. These experiments demonstrated that an automatic determination of BPEs in software imposes some fundamental challenges because the relationships between static and pulsatile pressure scores change constantly. These aspects are commented on after some addressing some aspects of single pressure wave (SW) analysis.

Measurements of SWs are needed for determination of pulsatile pressures and may also be used for determination of static mean pressure. The individual single pressure waves are created from the cardiac beat contractions and correspond to the pulse pressure. Various single pressure wave attributes are described in the international patent application WO 2006/009467 A2. Therefore, for the sake of clarity, some established single wave parameters are commented on, in particular single wave mean pressure (SW.meanP), single wave amplitude (SW.dP), single wave rise time (SW.RT) and single wave rise time coefficient (SW.RTC).

A cardiac-beat induced single (pulse) pressure wave is typically characterized by its beginning diastolic minimum pressure, systolic maximum pressure, and its ending diastolic minimum pressure. The mean single wave pressure (SW.meanP) represents a static pressure being absolute mean single wave pressure relative to a baseline pressure that is usually the atmospheric pressure. The mean single wave pressure (SW.meanP) may represent average of pressure samples divided by number of samples either during a rise time phase of the single pressure wave (SW.RT) or during an entire wave duration of the single pressure wave. The amplitude of the single pressure wave (SW.dP) is represented by differences in pressure between starting diastolic minimum pressures systolic maximum and systolic maximum pressure. Further, the single wave rise time coefficient (SW.RTC) is determined as the coefficient of single wave amplitude (SW.dP) over single wave rise time (SW.RT) according to the formula SW.RTC=SW.dP/SW.RT.

Furthermore, the patent WO 2006009467 A2 describes differences in single wave parameters between single waves may be denoted delta single wave (dSW.x) parameters. Some established dSW.x parameters are dSW.MeanP (=SWₙ.MeanP−SWₙ₋₁.MeanP), dSW.dP (=SWₙ.dP−SWₙ₋₁.dP), dSW.RT (=SWₙ.RT−SWₙ₋₁.RT), and dSW.RTC(=SWₙ.RTC−SWₙ₋₁.RTC). For example, a change in mean pressure (dSW.meanP) between single pressure waves represents change in absolute pressure between the single pressure waves, while a change in amplitude (dSW.dP) represents a change in amplitudes between single pressure waves.

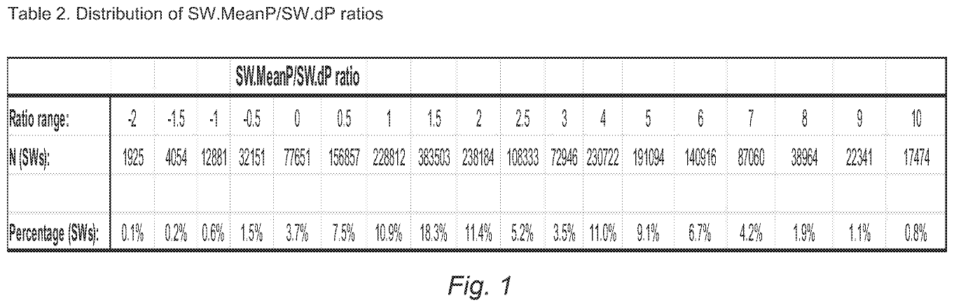

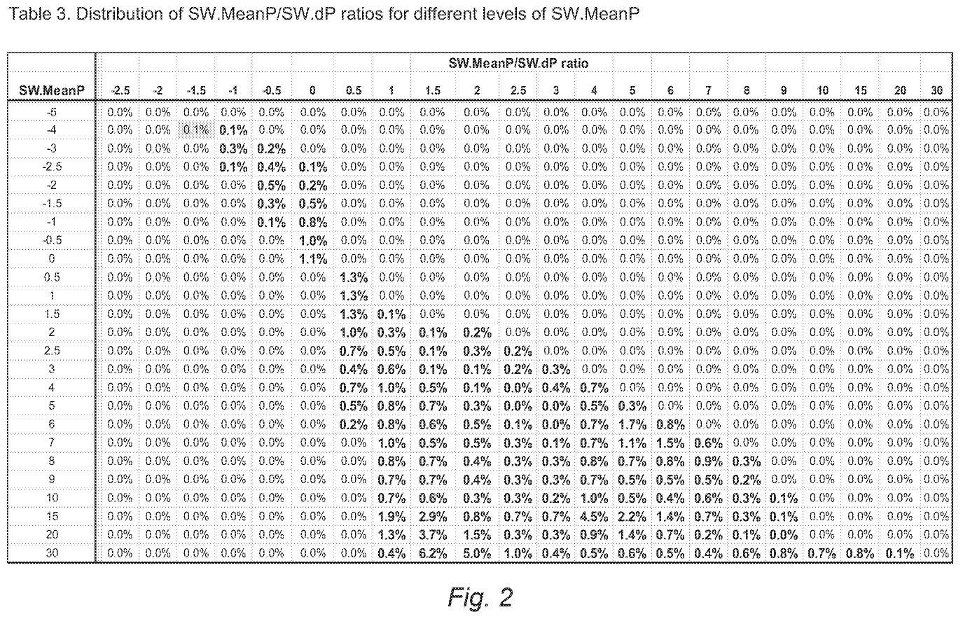

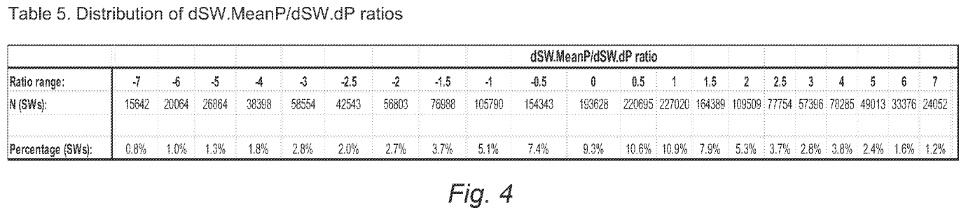

In some embodiments, baseline pressure changes from empirical observations of single wave (SW.x) parameters and delta single wave (dSW.x) parameters may be identified. To illustrate this, reference is made to a data material including 2,092,753 SWs from continuous invasive ICP measurements in individuals undergoing surveillance for intracranial bleeds. The ICP was recorded from the frontal brain region. Hence, the material is representative for continuous ICP measurements. Analysis of these observations shows how both single wave (SW.x) parameters and delta single wave (dSW.x) parameters distributed within certain thresholds. FIGS. 1-3 show Tables 2 to 4, respectively, which focus on SW.MeanP, SW.dP and SW.MeanP/SW.dP ratio. FIGS. 4-7 show Tables 5 to 7, respectively, which focus on dSW.MeanP, dSW.dP and the dSW.MeanP/dSW.dP ratio.

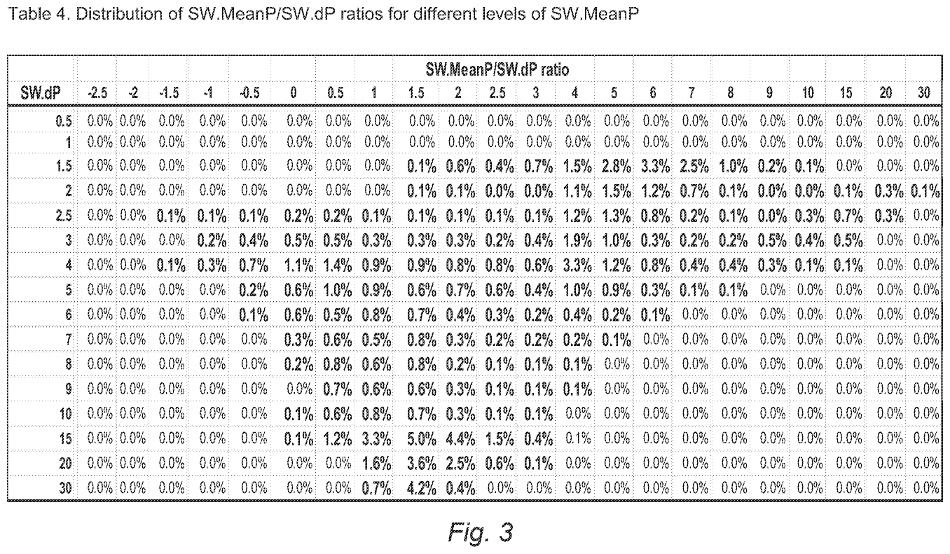

Table 2 of FIG. 1 shows part of a distribution of SW.MeanP/SW.dP ratios within the cohort of 2,092,753 single waves. The tabular presentation of the entire sample of 2,092,753 single waves revealed that 94% of ratio-observations were between −7 and +7, while 96% of ratio-observations were between −8 and +8. Table 3 of FIG. 2 shows a distribution of SW.MeanP/SW.dP ratio for different SW.MeanP levels, also based on the aforementioned cohort of 2,092,753 single waves. Total 94% of observations were within the cells with grey background. A SW.MeanP/SW.dP ratio of ≥7 was observed in 3.6% of observations when SW.MeanP was <10 mmHg, and in 5.2% of observations when SW.MeanP was <20 mmHg. A SW.MeanP/SW.dP ratio of ≥10 when SW.MeanP was <20 mmHg was not observed in this cohort. Table 4 of FIG. 3 shows the distribution of SW.MeanP/SW.dP ratio for different SW.dP levels in the cohort of 2,092,753 single waves. Total 99% of observations were within the cells with grey background. A SW.MeanP/SW.dP ratio of ≥7 was observed in 0.1% of observations when SW.dP was ≥5 mmHg. The observations presented in Tables 2 to 4 shown in FIGS. 1-3 illustrate the probability for the occurrence of certain SW.MeanP/SW.dP ratios for given thresholds of SW.MeanP and SW.dP. The empirical observations of Tables 2 to 4 in FIGS. 1-3 represent no limitation how criteria may be established for the identification of baseline pressure instability.

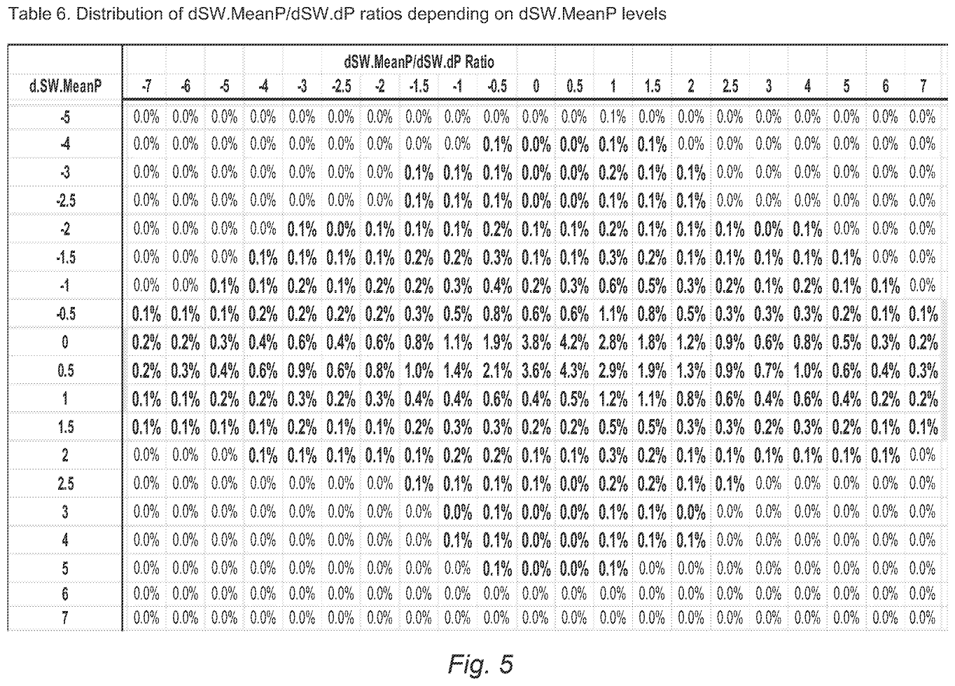

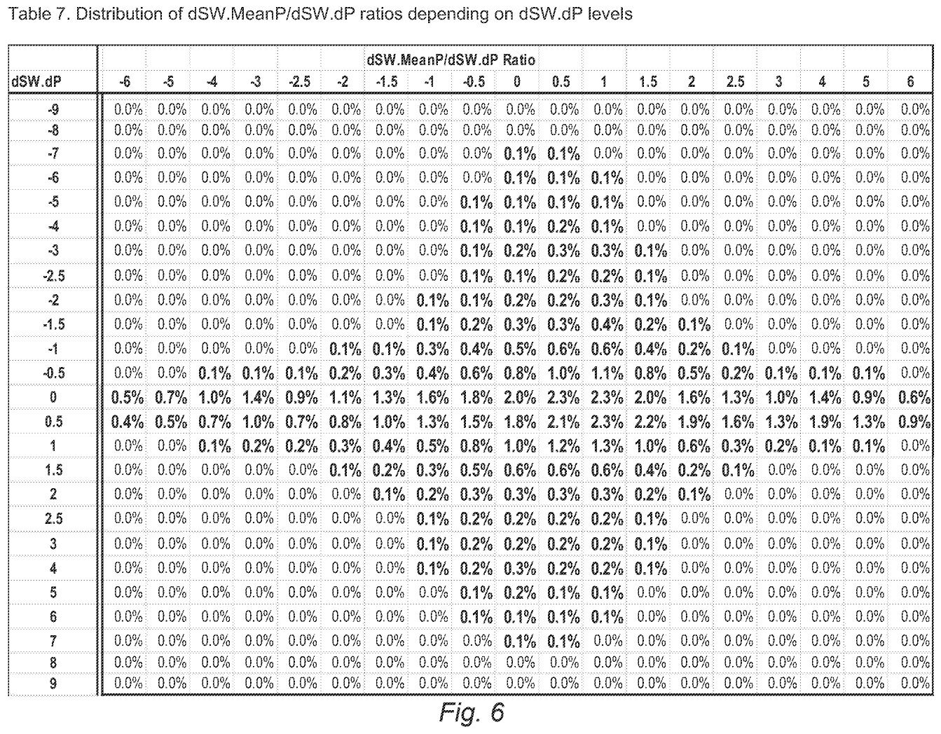

FIGS. 4-6 show Tables 5 to 7, respectively, which present information about the delta single wave (dSW.x) parameters dSW.MeanP versus dSW.dP. Table 5 in FIG. 4 shows part of a distribution of dSW.MeanP/dSW.dP ratios within the cohort of 2,092,753 single waves. The occurrences presented in Table 5 (shown in FIG. 4) revealed that 93% of ratio-observations were between −10 and +10. Table 6 of FIG. 5 shows how the dSW.MeanP/dSW.dP ratio distributes for different dSW.MeanP levels. Total 93% of observations were within the cells with grey background. A dSW.MeanP/dSW.dP ratio ≥7 when dSW.MeanP was ≥2 was not observed in this cohort. Each cell provides the percentage of occurrences. Table 7 of FIG. 6 shows how the dSW.MeanP/dSW.dP ratio distributes for different dSW.dP levels. Total 97% of observations were within the cells with grey background. A dSW.MeanP/dSW.dP ratio ≥2 when dSW.MeanP was ≥2 was observed in 0.1% within this cohort.

The tabular presentations demonstrate that there is a wide range of variation concerning SW.MeanP/SW.dP ratios and dSW.MeanP/dSW.dP ratios. In addition, automatic detection of dSW.MeanP/dSW.dP ratios has been created to establish information about baseline pressure changes. On this basis, the previous suggestions of determining the relationship between static and pulsatile pressure were found not to be useful for measuring baseline pressure instability.

After exploring a wide range of strategies to relate static and pulsatile pressure, it may be beneficial to develop other solutions for accurate assessment of baseline pressure instability of pressure sensors. Accordingly, this present disclosure provides technical solutions how to assess baseline pressure instability of pressure sensors used in humans. Moreover, the disclosure addresses how information about baseline pressure instability may be used for correction of static pressures.

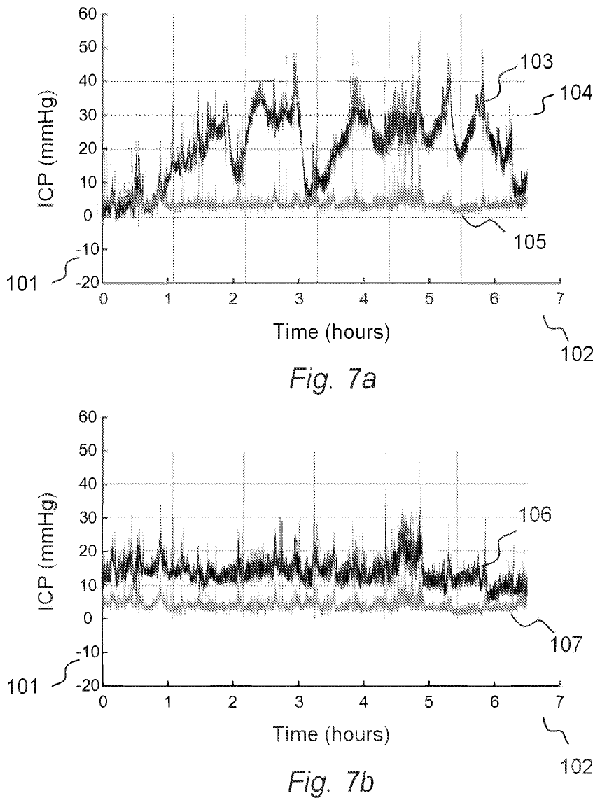

FIGS. 7a-b illustrates some important technical problems with today's practice of measuring static pressures; this case illustrates ICP monitoring. The ICP is usually measured from one single pressure sensor, commonly placed in the brain parenchyma or within the fluid of the ventricular system. In a few instances, the ICP has been measured from two different ICP sensors placed nearby within the brain. These special cases are particularly interesting as they allow for comparisons between different ICP sensors. It would be expected that measurements from two different ICP sensors placed nearby should provide similar ICP scores, given that the ICP sensors are placed so close that no pressure gradients exist. FIGS. 7a-b presents the ICP measurements from two different ICP sensors in a patient who had two ICP sensors nearby in the right frontal lobe of the brain, namely a Camino ICP sensor (FIG. 7a) and a Codman ICP sensor (FIG. 7b). At the point of time when the ICP measurement was done, the patient was sedated and on artificial ventilation. In that situation, the ICP measurements are crucial for surveillance of the patient. Typically, the aim is to keep mean ICP below 20 mmHg. If ICP raises above 20 mmHg, actions may be performed, which include providing medication to reduce ICP, drainage of cerebrospinal fluid, or even surgical procedures. In this case, the mean ICP measured from the two ICP sensors were very different even though they measured ICP from the same site in the brain. In other words, one or both ICP sensors gave erroneous ICP scores.

In FIG. 7a-b, the y-axis 101 presents ICP in mmHg, and the x-axis 102 the time shown in hours. This ICP recording lasted about 6.5 hours. The Camino ICP sensor (FIG. 7a) consistently showed a very high mean ICP score, as depicted in the trend plot of mean ICP 103. The average of mean ICP was 20.6 mmHg, but the horizontal dotted line 104 at 30 mmHg shows that mean ICP was above 30 mmHg at several occasions. The trend plot of (mean ICP wave amplitude) MWA 105 was more stable and had an average of 4.3 mmHg. In comparison, the mean ICP measured simultaneously from the Codman ICP sensor from the same site as the Camino ICP sensor was very different, as revealed by the trend plot of mean ICP 106 (FIG. 7b). The average of mean ICP measured from the Codman ICP sensor was 14.1 mmHg. Since mean ICP above 20 mmHg as the threshold for intervention, this patient could have been given very different treatment while being in the exact same sate. The trend plot of MWA for the Codman sensor 107 illustrates that MWA measured by the Codman ICP sensor (average 4.5 mmHg) was, however, close to identical to the MWA of the Camino ICP sensor (4.3 mmHg) for the two recordings. Several questions arise from this observation, including which mean ICP is correct, that of the Camino (FIG. 7a) or that of the Codman (FIG. 7b)/There are currently no measures to check which ICP measurement is correct. Even though the MWA is close to identical for the two measurements, the mean ICP may differ extensively. This observation among others was a motivation to develop technical solutions to solve these issues.

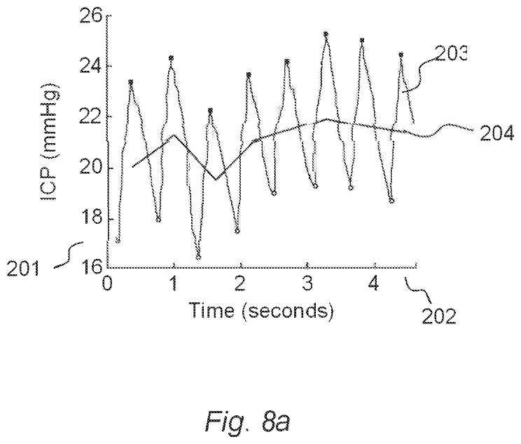

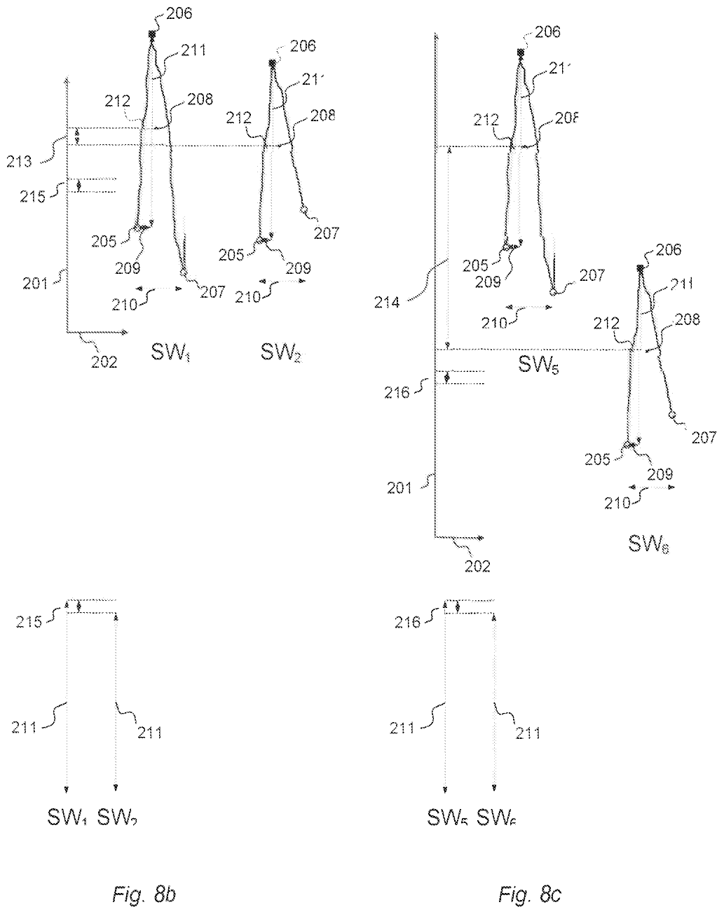

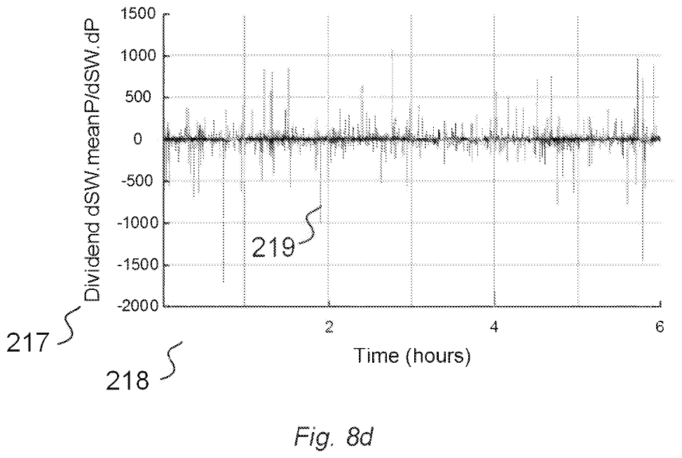

The single pressure wave (SW.x)-related parameters and delta single pressure wave delta (dSW.x) parameters are briefly commented on with reference to FIG. 8a-b. FIG. 8a illustrates that a pressure signal is described by two dimensions, namely by pressure level 201 and time 202. The individual single waves 203 are created from the cardiac beat contractions and correspond to the pulse pressure. Plotting of mean single wave pressure (SW.MeanP) 204 over time is shown. During the pressure monitoring, both the single pressure waves 203 and the mean single wave pressure 204 fluctuate.

With reference to FIGS. 8b-c, a cardiac-beat induced single (pulse) pressure wave 203 is typically characterized by its beginning diastolic minimum pressure 205, systolic maximum pressure 206, and its ending diastolic minimum pressure 207. The mean single wave pressure (SW.meanP) 208 represents a static pressure being absolute mean single wave pressure relative to a reference pressure that is usually the atmospheric pressure. The mean single wave pressure (SW.meanP) 208 may represent average of pressure samples divided by number of samples either during a rise time phase of the single pressure wave (SW.RT) 209 or during an entire wave duration of the single pressure wave (SW.WD) 210. While the rise time (SW.RT) 209 refers to the time elapsed from the beginning diastolic minimum pressure 205 to the systolic maximum pressure 206, the wave duration (SW.WD) 210 refers to the time elapsed from starting diastolic minimum pressure 205 to ending diastolic minimum pressure 207. When mean single wave pressure (SW.meanP) 208 is the average of pressure samples divided by number of samples during an entire wave duration of the single pressure wave (SW.WD) 210, it is similar to the area under curve for the single pressure wave (SW. AUC). The amplitude of the single pressure wave (SW.dP) 211 is represented by differences in pressure between starting diastolic minimum pressures systolic maximum 205 and systolic maximum pressure 206. Further, the single wave rise time coefficient (SW.RTC) 212 is determined as the coefficient of single wave amplitude (SW.dP) 211 over single wave rise time (SW.RT) 209 according to the formula SW.RTC=SW.dP/SW.RT.

FIG. 8b and FIG. 8c illustrate that single pressure waves may be comparable even though mean single wave pressure changes markedly. The change in mean single wave pressure (dSW.meanP) 213 between single waves 1 (SW₁) and 2 (SW₂) is modest (FIG. 8b), while the change in mean single wave pressure (dSW.meanP) 214 between single waves 5 (SW₅) and 6 (SW₆) is marked (FIG. 8c). In comparison, the change in amplitude (dSW.dP) 215 between single waves 1 (SW₁) and 2 (SW₂) and change in amplitude (dSW.dP) 216 between single waves 5 (SW₅) and 6 (SW₆) are comparable. A change in mean pressure (dSW.meanP) between a consecutive number of single pressure waves represents change in absolute pressure between the single pressure waves. For example, delta mean pressure (dSW₂₋₁.meanP) 213 represents the difference SW₂.meanP of SW₂ minus SW₁.meanP of SW₁ (FIG. 8b). In comparison, delta mean pressure (dSW₆₋₅.meanP) 214 represents the difference SW₆.meanP of SW₆ minus SW₅.meanP of SW₅ (FIG. 7c). Delta amplitude (dSW₂₋₁.dP) 215 represents the difference SW₂.dP of SW₂ minus SW₁.dP of SW₁ (FIG. 8b), and delta amplitude (dSW₆₋₅.dP) 216 represents the difference SW₆.dP of SW₆ minus SW₅.dP of SW₅ (FIG. 8c).

The delta single wave parameters may be determined for all single wave parameters. For example, a change in amplitude (dSW.dP) 215, 216 between a selectable number of single pressure waves (n−1; n) represents change in internal signal relative pressure between the single pressure waves. Differences may be determined between subsequent single waves (SWₙ₋₁.x versus SWₙ.x) or between any selected single waves in a series of multiple single waves (SWₙ₋₁.x versus SWₙ.x). The single waves that are compared represent no limitation of the disclosure. FIG. 8b and FIG. 8c illustrate some single wave (SW.x) and delta single wave (dSW.x) parameters, which also are listed in Table 8.

TABLE 8
Important SW.x and dSW.x parameters used for this disclosure.

| SW-parameters | dSW-parameters |
|---|---|
| SW.MeanP | dSW.MeanP (= SWn.MeanP − SWn-l.MeanP) |
| SW.dP | dSW.dP (= SWn.dP − SWn − i.dP) |
| SW.RT | dSW.RT (= SWn.RT − SWn − i.RT) |
| SW.RTC | dSW.RTC (= SWn.RTC − SWn − i.RTC) |
| SW.WD | dSW.WD (= SWn.WD − SWn − i.WD) |
| SW.AUC | dSW.AUC (= SWn.AUC − SWn − i.AUC) |

From Reference 3), it was suggested to determine how mean ICP and mean ICP wave amplitude relates during ongoing monitoring, i.e. a sudden change in mean ICP not accompanied by a change in the ICP wave amplitude should alert the clinician to a technical rather than a biological problem. From this, one strategy would be to plot the change in mean pressure (dSW.meanP) together with change in amplitude (dSW.dP). This option was explored in experiments, which is shortly commented on. With reference to FIG. 8b, the determination of the dividend between dSW.meanP 213 and dSW.dP 215 between the single pressure waves SW₂ and SW₁ is described in brief. For the first single wave (SW₁), FIG. 8b illustrates the mean pressure 208 and the amplitude 211, and similarly for the second single wave (SW₂), the mean pressure 208 and amplitude 211. It is illustrated that along the pressure scale 201 the change in mean pressure dSW.meanP 213 and change in amplitude dSW.dP 215 between single waves SW₁ and SW₂. The dividend between dSW.meanP 213 dSW.dP 215 is determined between change in mean pressure 213 and change in amplitude 215. FIG. 8d shows a plot of dSW.meanP/dSW.dP values 217 against time 218. In this example, the dSW.meanP/dSW.dP values 217 are determined from consecutive SWs. Each vertical spike 219 shows over time the magnitude of dSW.meanP/dSW.dP. For example, dSW.meanP/dSW.dP values outside thresholds of +/−100 are far from thresholds established according to empirical data (Tables 6 and 7 of FIGS. 5 and 6, respectively) and may represent baseline pressure instabilities. The plot shown in FIG. 8d shows a recording with a high proportion of dSW.meanP/dSW.dP values of large magnitudes. According to experiments, this kind of plot might not be useful for presenting instability of baseline pressure. This is because plots like this vary extensively between pressure recordings. While some recordings have limited frequency of dSW.meanP/dSW.dP of large values, other pressure recordings like that illustrated in FIG. 8d have higher frequency of dSW.meanP/dSW.dP values with larger magnitude. However, while the plot shown in FIG. 8d includes the time dimension 218, this plot does not show whether the dSW.meanP/dSW.dP value 217 results in a lasting change in the SW.meanP/SW.dP relationship. Therefore, plotting of relationship between static and pulsatile pressure as dividend between change in single wave mean pressure (dSW.meanP) and change in single wave amplitude (dSW.dP) might not be useful to visualize instability of baseline pressure.

From these experiments, it became clear that determining the relationship between change in mean pressure and pressure amplitude could not be used provide information about baseline pressure. Other technical solutions were implemented that led to performing different experiments. Since the report (Reference 3), this issue has not been addressed in the literature. Hence, reviewing the existing literature indicates that issues related to baseline pressure instability are considered less important. Technical solutions based on experiments and implementation of test software were developed. Test studies of numerous real continuous pressure recordings were done to test the technical solutions.

Novel steps that resulted from the experimental studies are now commented on in more detail. One step includes calculation of pressure stability levels (SW.x.PSL) and determination of pressure differences between different pressure stability levels (SW.x.PSL.PD), which together form basis for creating of baseline pressure indicator (BPi) plots. The pressure stability levels have definable duration (SW.x.PSL.TD) relating to the time duration of the pressure stability levels (SW.x.PSL). This time duration also refers to how many observations of delta single wave (dSW.x) parameters that are included in a pressure stability level (SW.x.PSL). Accordingly, a SW.x.PSL.TD may either refer to the time elapsed from the beginning to the ending part of a pressure stability level, or it may refer to the number of observations of delta single wave (dSW.x) parameters included in a pressure stability level. In test software, number of observations of delta single wave (dSW.x) parameters for defining duration of a pressure stability level (SW.x.PSL.TD) were applied. Additional steps were developed to assess pressure sensor instability and related instability of baseline pressure from the baseline pressure indicator (BPi) plots. According to the disclosure, the indication of baseline pressure instability could be used for correction of mean pressure measurements.

The notation “indicator plot” is used because the baseline pressure itself may hardly be determined exactly in vivo without removing the pressure sensor and measure against a zero pressure (atmospheric pressure). Moreover, in some situations it may be difficult to define whether apparent baseline pressure instability is related to physiological alterations or alternatively being related to technical flaws or functional instability of the pressure sensor. However, based on the presently described steps, the information provided by baseline pressure indicator plots may with high probability define baseline pressure instability of a pressure sensor, independent of the cause. The baseline pressure indicator plot provides for continuous scoring of baseline pressure instability. It may be implemented as a baseline pressure instability detector in software or integrated in a system or apparatus for pressure monitoring. Alternatively, it may be integrated in presently used pressure sensors (second use). Analysis may be done “on the fly” or pressure measurements may be checked after monitoring has been ended. Different kinds of analyses are possible, for example as “background analysis” checking previous monitoring.

In some cases, it might not be feasible to create a baseline pressure indicator plot simply from plotting the single wave parameters. It would be impossible to define the average pressure value and its duration. What should be the criteria for which pressure values that should be included in the plot? Furthermore, implementation in test software and analysis of empirical observations were implemented to establish the steps of calculating pressure stability levels (SW.x.PSL) and determining pressure differences (SW.x.PSL.PD), which together create the baseline pressure indicator plots that incorporate information about stability of baseline pressure.

Test software was implemented to test calculations of pressure stability levels and determination of pressure differences between the pressure stability levels. Three different types of novel thresholds (threshold types one, two and three) were determined, based on assessing pressure measurements. The software had to be tested on real continuous pressure recordings to see its behavior and impact of threshold setting. Hence, empirical observations were implemented. Moreover, it was needed to apply test software to pressure measurements with verified instability of baseline pressure. Finally, automation of methods and systems for providing information about stability of baseline pressure of a pressure sensor made the manual inspection of pressure readings unnecessary.

Current systems and methods do not create baseline pressure indicator plots from pressure stability levels and pressure differences between different pressure stability levels established to assess baseline pressure instability of a pressure sensor. Neither do they use second and third types of thresholds to define instability of baseline pressure from baseline indicator plots. In the following paragraphs, the steps of determining baseline pressure instability of pressure sensors are described in detail.

In the following, calculation of pressure stability levels (SW.x.PSL) are described in more detail. These pressure stability levels are one important ingredient of the baseline pressure indicator (BPi) plots. The pressure stability levels are calculated from single pressure wave (SW.x)-related parameters, incorporating a consecutive number of single pressure waves with delta single pressure wave (dSW.x)-related parameters within a first type of selectable thresholds. As already commented on, delta single pressure wave (dSW.x)-related parameters are computed from differences in single pressure wave (dSW.x)-related parameters. In the first experiments, change in mean pressure (dSW.meanP), change in amplitude (dSW.dP), change in rise time (dSW.RT) and change in rise time coefficient (dSW.RTC) were the focus, even though the type of delta single wave (dSW.x)-related parameter (see FIGS. 7A-7B) does not represent a limitation of the disclosure.

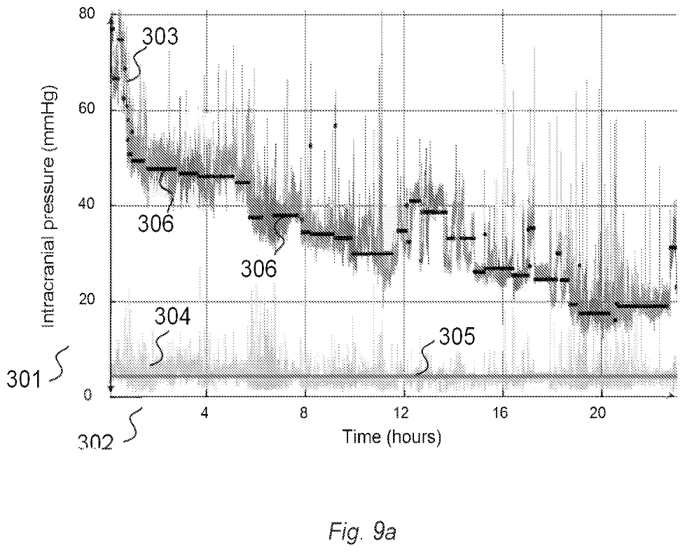

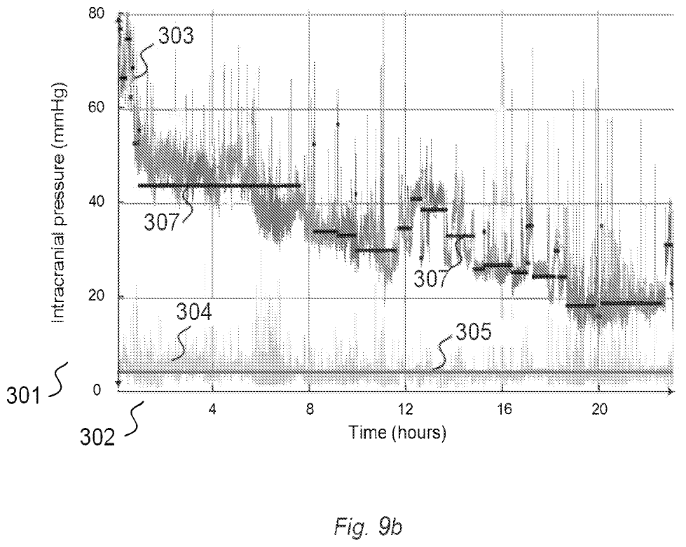

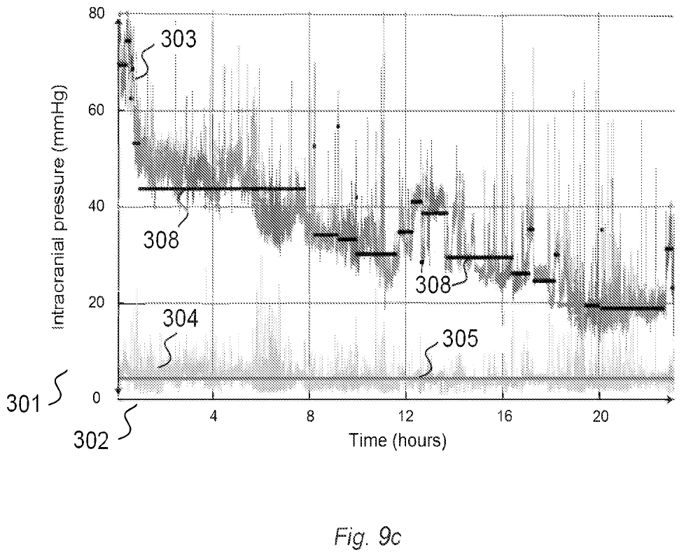

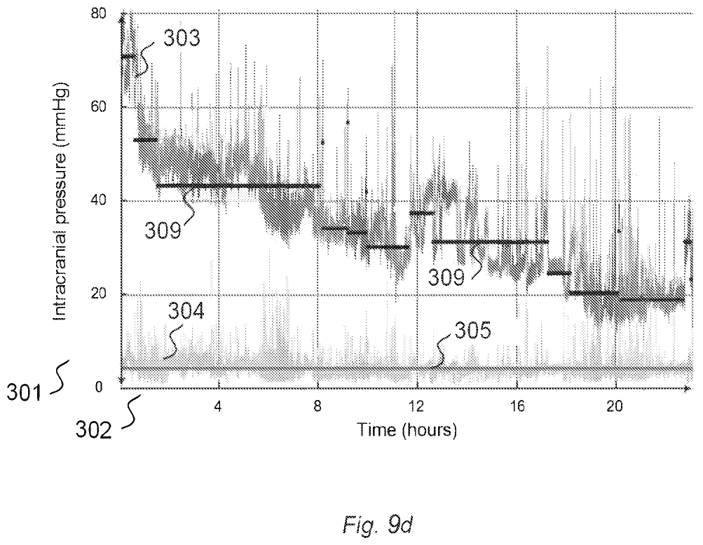

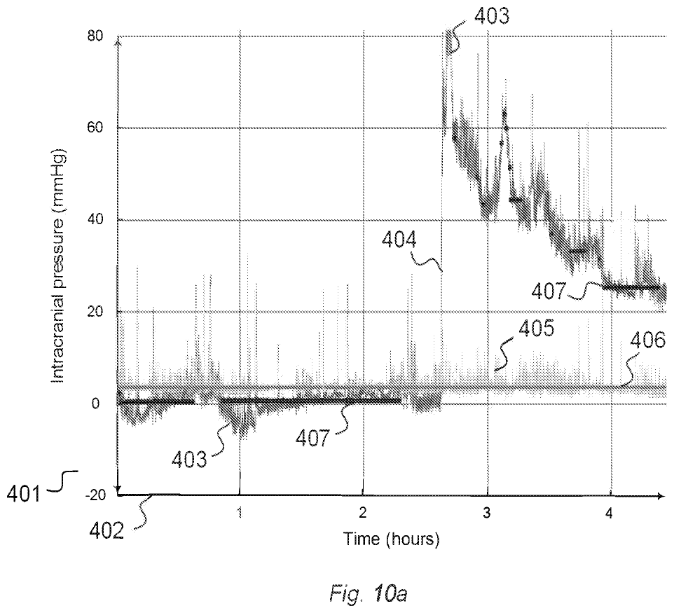

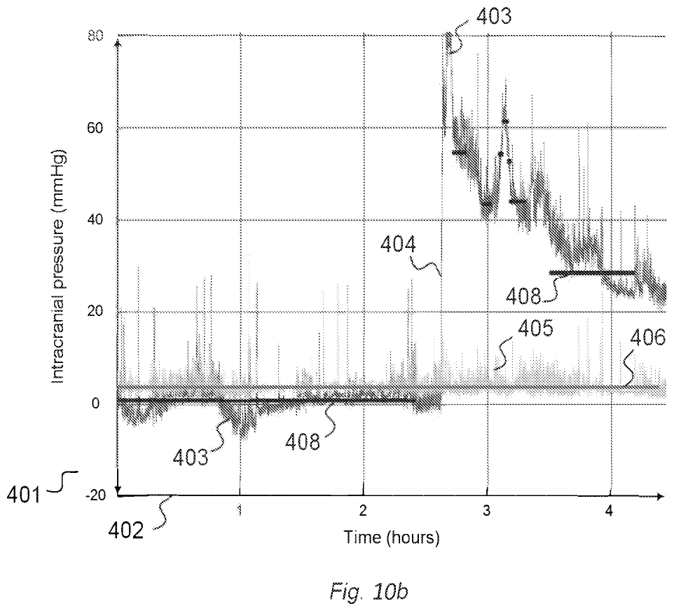

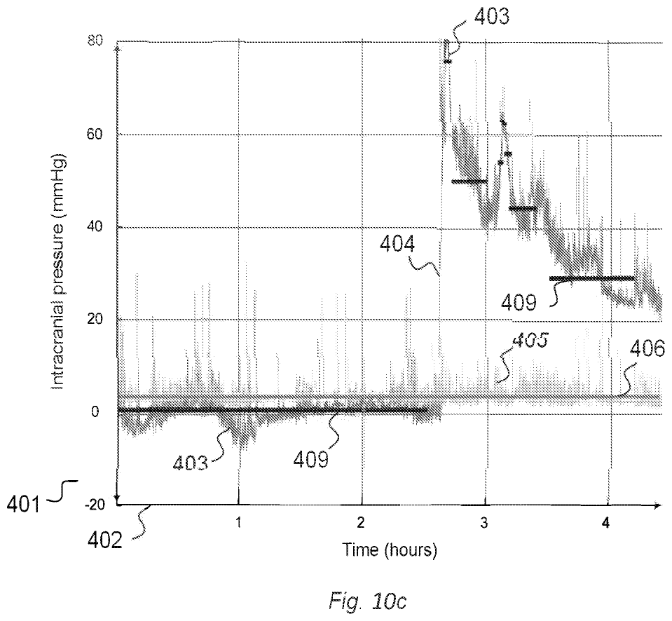

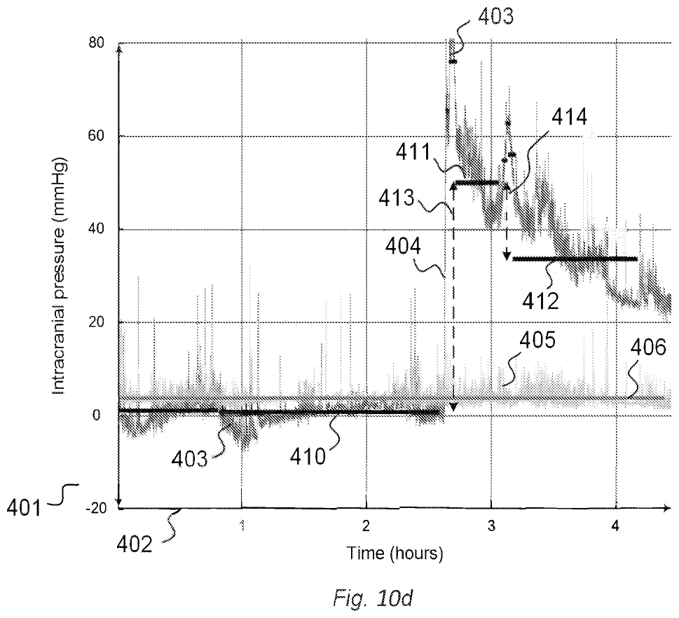

The methodology for calculating pressure stability levels (SW.x.PSL) incorporates the following: Single waves (SWs) with delta single wave dSW.x) related parameters within selectable thresholds are included in the given pressure stability level. The single pressure waves included in the pressure stability level may be based on either of the respective delta single pressure wave parameters dSW.meanP, dSW.dP and dSW.RTC. A pressure stability level (SW.x.PSL) refers to the average value of either of the single pressure wave parameters SW.meanP, SW.dP and SW.RTC, thus referring to the parameters SW.meanP.PSL, SW.dP.PSL and SW.RTC.PSL, respectively. Moreover, a defined pressure stability level includes single waves with dSW.x parameters within a First type of selectable thresholds. There is no limitation how the selectable thresholds are defined. For example, concerning a pressure stability level of mean ICP (ICP.SW.meanP.PSL), consecutive single ICP waves are added to the same pressure stability level as long as dSW.meanP is within ranges of a first type of thresholds. When dSW.meanP deviates from the first type of thresholds, a next pressure stability level is created incorporating the subsequent single waves having dSW.meanP within the first type of thresholds. Thereby, in this example, a pressure stability level represents the average value of ICP.SW.meanP of the pressure stability level. In an automatized method, the first type of threshold may be adjustable during ongoing monitoring. To further illustrate how the First type of thresholds impact pressure stability levels, results of implementation in test software are illustrated in FIGS. 9a-9d and 10a-10d. Pressure stability levels are calculated automatically for the single wave parameters mean ICP (SW.meanP) and amplitude (SW.dP). For sake of clarity, it is particularly focused on pressure stability levels for SW.meanP, i.e. SW.meanP.PSL.

FIGS. 9a-9d and 10a-10d illustrate how the pressure stability levels (SW.x.PSL) depend on the first type of thresholds that are used.

FIG. 9a-9d presents an ICP recording with a possible spontaneous change in baseline pressure, and illustrates how the pressure stability levels are somewhat modified depending on the thresholds of dSW.meanP. The y-axis shows the ICP 301 and the x-axis the time 302. The mean ICP (SW.meanP) 303 is plotted against time and shows a gradual reduction over time. Moreover, the amplitude (SW.dP) 304 is plotted against time; the pressure stability level for single wave amplitude (SW.dP.PSL) 305 was stable and unchanged during the entire measurement period. With reference to FIG. 9a, the first type of thresholds for dSW.meanP included in a pressure stability level (SW.meanP.PSL) were:

1) dSW.meanP <4 mmHg,

2) number of included samples (i.e. dSW.meanP observations) per pressure stability level (i.e. minimum dSW.meanP observations) >40, and

3) merging nearby pressure stability levels (SW.meanP.PSLₙ₋₁ versus SW.meanP.PSLn) with a pressure difference <4 mmHg (i.e. SW.meanP.PSL.PD <4.0 mmHg). The latter refers to a criterion where nearby pressure stability levels are merged into one pressure stability level if pressure difference between pressure stability levels (SW.x.PSL.PD) is within selectable thresholds. Applying the above-mentioned criteria gave pressure stability levels (SW.meanP.PSL) illustrated by black horizontal lines 306. As can be seen, using these first type of thresholds, a number of pressure stability levels 306 were identified.

In FIG. 9b, a first type of threshold in test software defining the dSW.meanP observations to be included in a pressure stability level group is dSW.meanP <5 mmHg, and the pressure-difference threshold for merging nearby pressure stability levels (SW.x.PSL.PD) is 5 mmHg. In other words, one pressure stability level (SW.meanP.PSL) 307 includes minimum 40 samples of dSW.meanP with value <5 mmHg. Since nearby pressure stability levels 307 with a pressure difference (SW.meanP.PSL.PD)<5 mmHg were merged, the duration of pressure stability levels (SW.x.PSL.TD) was of somewhat longer duration (SW.meanP.PSL.TD) than with thresholds used in FIG. 9a.

In FIG. 9c, the first type of thresholds were changed as compared to illustrations of FIGS. 9a-b. The pressure stability levels 308 of FIG. 9c were calculated from inclusion of dSW.meanP values of <6 mmHg, and the threshold for merging nearby pressure stability levels (SW.x.PSL.PD) is 6 mmHg, i.e. some Thereby, one pressure stability level (SW.meanP.PSL) 308 includes minimum 40 samples of dSW.meanP with value <6 mmHg, and with merge of nearby pressure stability levels with a pressure difference <6 mmHg. In comparison, for the pressure stability levels 309 shown in FIG. 9d, the threshold for dSW.meanP is 8 mmHg, and the threshold for merging nearby pressure stability levels is 8 mmHg. Accordingly, one pressure stability level (SW.meanP.PSL) 309 includes minimum 40 samples of dSW.meanP with value <8 mmHg. In addition, nearby pressure stability levels with a pressure difference <8 mmHg were merged.

Taken together, the pressure stability levels 306, 307, 308 and 309 of FIGS. 9a-d differ somewhat depending on the selectable first type of thresholds used. Both the magnitude of pressure stability level (SW.x.PSL) and duration of each pressure stability level (SW.x.PSL.TD), i.e. the definable duration of the pressure stability level, depend on the first type of thresholds used. In general, the more strict criteria used, a lower number of pressure stability levels are created. In the first test experiments, the following first type of thresholds were found to be most useful for calculation of pressure stability levels for mean intracranial pressure (ICP.SW.meanP.PSL):

1) ICP.dSW.meanP <5 mmHg (ranges 3-5 mmHg),

2) ICP.dSW.meanP.PSL.PD <5 mmHg for merge of nearby pressure stability levels (ranges 3-5 mmHg), and

3) minimum samples within a pressure stability level >40 (ranges 30-50).

While these specific thresholds represent no limitation of the disclosure, they were useful for testing the ability of this step to assess baseline pressure instability of a pressure sensor used in humans. Moreover, while FIGS. 9a-d, show pressure stability levels of the parameters SW.meanP and SW.dP, comparable pressure stability levels may be presented for other SW.x parameters, such as SW.RTC. In some cases, the exact threshold levels depend on the pressure in question. For example, thresholds are different for intracranial pressure (ICP) and arterial blood pressure (ABP).

To further illustrate the use of first type of thresholds for creation of pressure stability levels, another example is given in FIG. 10a-d. The test software was applied to an ICP recording incorporating a sudden shift in mean pressure (ICP.SW.meanP). Why such a sudden change in mean pressure occurs cannot be determined with certainty, but most likely refers to a baseline pressure shift caused by instability of the pressure sensor. Hence, a sudden change in baseline (or reference) pressure of the ICP sensor due to technical reasons may be seen as a sudden change in mean ICP. The impact of applying different selectable first type of thresholds for calculation of pressure stability levels is illustrated. Referring to FIG. 10a-d, the y-axis shows the ICP 401 and the x-axis the time 402. The mean ICP (SW.meanP) 403 is plotted against time. The occurrence of a baseline pressure shift 404 is shown by a straight line on the mean ICP (SW.meanP) plot. The single wave amplitude (SW.dP) 405 is plotted against time and the single wave amplitude pressure stability level (SW.dP.PSL) 406 is stable and unchanged during the entire measurement period, but is not further commented on in this context.

In FIGS. 10a-d, different first type of thresholds were used to determine which dSW.meanP values that were included in a pressure stability level (SW.meanP.PSL), also indicating merge of nearby pressure stability levels (SW.meanP.PSLₙ₋₁ versus SW.meanP.PSLn). For all plots in FIGS. 10a-d, the minimum number of samples (dSW.meanP observations) within a pressure stability level was kept unchanged, that is, >40. In FIG. 10a, the threshold for dSW.meanP is 3 mmHg, and the threshold for merging nearby pressure stability levels 3 mmHg. Thereby, one pressure stability level (SW.meanP.PSL) 407 includes minimum 40 samples of dSW.meanP with value <3 mmHg, and wherein nearby pressure stability levels with a pressure difference <3 mmHg were merged. In FIG. 4b, the threshold for dSW.meanP was 4 mmHg, and the threshold for merging nearby pressure stability levels was 4 mmHg. Hence, one pressure stability level (SW.meanP.PSL) 408 includes minimum 40 samples of dSW.meanP with value <4 mmHg. Nearby pressure stability levels with a pressure difference (dSW.meanP.PD)<4 mmHg were merged. In FIG. 10c, the threshold for dSW.meanP is 5 mmHg, and the threshold for merging nearby pressure stability levels is 5 mmHg. Thereby, one pressure stability level (SW.meanP.PSL) 409 includes minimum 40 samples of dSW.meanP with value <5 mmHg, and nearby pressure stability levels with a pressure difference <5 mmHg were merged. Finally, for the pressure stability levels shown in FIG. 10d, the threshold for dSW.meanP is 6 mmHg and the threshold for merging nearby pressure stability levels 6 mmHg. In FIG. 1d, three pressure stability levels are marked separately, namely 410 occurring before the shift 404, and 411 and 412 occurring after 404. Also in this example, including a first type of threshold with dSW.mean P <4 mmHg or <5 mmHg, seemed most useful, though the exact threshold used is selectable.

A pressure stability level is built up of a consecutive number of single waves with delta single wave parameters (dSW.x) within a first type of selectable thresholds. The time duration of a pressure stability level (SW.x.PSL.TD) is defined by the number of included delta single wave parameters (dSW.x). In test software, a number of observations of delta single wave (dSW.x) parameters were applied for defining duration of a pressure stability level (SW.x.PSL.TD). This way of defining duration of a pressure stability level represents no limitation of the disclosure. As illustrated in FIG. 10a-d, the time duration of a pressure stability level (SW.x.PSL.TD) varies substantially, depending how many delta single wave (dSW.x) observations that are included in a pressure stability level, as exemplified for the pressure stability levels 407, 408, 409, 410, 411, and 412. Moreover, since consecutive pressure stability levels may be merged into one if the pressure difference between consecutive pressure stability levels is within selectable thresholds, the pressure stability level in FIG. 10a-d of single wave amplitude (SW.dP.PSL) 305 is at the same level, and also at the same level 406 in FIG. 10a-d.

A baseline pressure indicator (BPi) plot is created from pressure stability levels (SW.xPSL) and with beginning and ending pressure differences (SW.x.PSL.PD) for each pressure stability level (SW.x.PSL). Hence, for one individual pressure stability level, there is a pressure difference both at the start and at the end of one individual pressure stability level. The beginning pressure difference is defined as the difference between a present and a previous pressure stability level and the ending pressure difference is defined as the difference between a present and a next pressure stability level. To further illustrate, in FIG. 10d, three pressure stability levels are indicated, namely 410, 411 and 412. For pressure stability level (SW.x.PSL) 411, is shown the beginning pressure difference (beginning SW.x.PSL.PD) 413 and the ending pressure difference (ending SW.x.PSL.PD) 414.

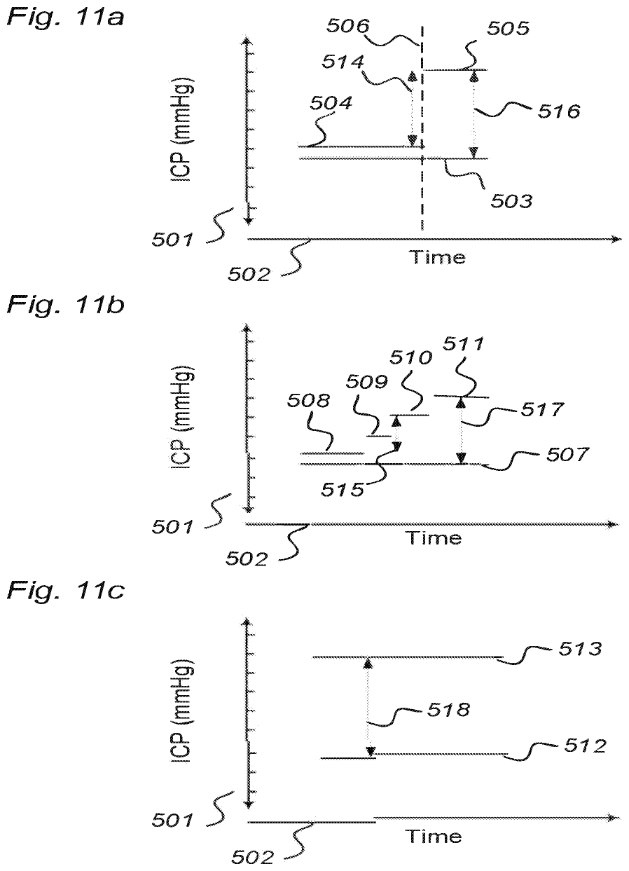

Examples of creations of baseline pressure indicator (BPi) plots are illustrated in FIGS. 11a-c, 12a-c, 13a-c and 14a-c. Information from the baseline pressure indicator (BPi) plots defines baseline pressure instability of a pressure sensor by incorporating further steps, namely a) secondary type of threshold and b) third type of thresholds. This latter aspect is illustrated schematically in FIG. 11a-c. In some embodiments, first, second, and third types of thresholds may be referred to herein as first, second, third sets of thresholds, respectively.

FIG. 11a-c shows schematically three different examples of pressure stability levels (SW.x.PSL) for different single wave parameters (SW.x), and illustrate assessment of a) individual pressure stability levels calculated from the same type of single pressure wave (SW.x)-related parameters, and b) pressure stability levels from different and simultaneous pressure stability levels calculated from different single pressure wave (SW.x)-related parameters. The disclosure refers to Second and Third types of thresholds, respectively. The pressure stability levels shown in FIG. 11a-c refer to ICP.SW.meanP and ICP.SW.dP, though this should not limit the disclosure, as any single wave parameters may be compared. The ICP in mmHg is plotted on the y-axis 501 and the time of recording on the x-axis 502. With reference to FIG. 11a, the pressure stability level 503 for single wave amplitude (SW.dP.PSL) was stable and unchanged during the entire measurement period. With regard to the single wave parameter SW.meanP, there is a marked change between a first pressure stability level 504 and a second pressure stability level 505. The point of time 506 where an abrupt change occurs in pressure stability level (SW.meanP.PSL) is indicated. It should be noted that both the SW.meanP and SW.dP are derived from the same pressure sensor and the same continuous pressure signal. In other words, at a certain point of time 506, there is a sudden change in single wave mean pressure (SW.meanP) that is not accompanied with a change in single wave amplitude. In FIG. 11b, the pressure stability level for single wave amplitude (SW.dP.PSL) 507 also is stable during the measurement period. For single wave mean pressure (SW.meanP), on the other hand, four pressure stability levels (SW.meanP.PSL) 508, 509, 510, and 511 are shown, Hence, there is a gradual change in pressure stability level for mean pressure (SW.meanP.PSL) 508, 509, 510, and 511 even though this is not accompanied with a change in stability pressure level for single wave amplitude (SW.dP.PSL) 507. Again, both mean pressure (SW.meanP) and amplitude (SW.dP) are derived from the same pressure sensor and the same continuous pressure signal. Another situation is presented in FIG. 11c; both the pressure stability level of single wave amplitude (SW.dP.SPL) 512 and mean pressure (SW.meanP.PSL) 513 are stable.

The Second type of thresholds refer to assessing individual pressure stability levels calculated from the same type of single pressure wave (SW.x)-related parameters. Therefore, applying second type of thresholds enable defining baseline pressure instability when measuring only one single wave-related parameter, e.g., only measuring mean ICP. In FIG. 11a, the second type of thresholds define which threshold for change in pressure (ICP.SW.meanP.PSL.PD) 514 between pressure stability levels 504 and 505 that are outside or inside defined threshold ranges. Similarly, in FIG. 11b, the second type of thresholds define which threshold for change in pressure (ICP.SW.meanP.PSL.PD) 515 between pressure stability levels 509 and 510 (or between any other pressure stability level of SW.meanP) that are outside or inside defined threshold ranges. For example, the combinations of pressure differences between the pressure stability levels (SW.meanP.PSL) 504 and 505, indicated as 514, and between 510 and 508, indicated as 515, are outside or inside the second type of thresholds depending how pressure differences deviate from nominal reference pressure differences. Combinations of pressure differences outside the second type of thresholds may determine whether a baseline indicator plot defines instability of baseline pressure of a pressure sensor. In this regard, it should be noted that the second type of thresholds refer to pressure differences (SW.x.PSL.PD) between pressure stability levels of definable time durations (SW.x.PSL.TD). As such, the second type of thresholds incorporate a time dimension in addition to a pressure difference dimension.

Nearby pressure stability levels (SW.x.PSL) may be merged into one pressure stability level (SW.x.PSL) if pressure differences between pressure stability levels (SW.x.PSL.PD) are within selectable ranges of the second type of selectable thresholds. It was illustrated in FIGS. 9a-d and 10a-d that selectable thresholds for pressure difference between pressure stability levels (SW.x.PSL.PD) determine whether pressure stability levels may be merged. In some cases, the latter heavily impacts the time duration of a pressure stability level (SW.x.PSL.TD). In addition to the pressure difference per se, another set of criteria define that pressure differences (SW.x.PSL.PD) are only determined for pressure stability levels incorporating a minimum number of observations. This is because it may not be useful to determine pressure differences for pressure stability levels of short durations. For example, FIG. 6a shows many pressure stability levels, and it might not be preferable to determine pressure difference between all these pressure stability levels. Likewise, in FIG. 10c, there are several pressure stability levels 409 after the pressure shift 404.

On the other hand, the Third type of thresholds refer to relationships between different and simultaneous pressure stability levels calculated from different types of single pressure wave (SW.x)-related parameters. This aspect is illustrated in FIG. 11a, where the relationship 516 between pressure stability level of mean ICP (ICP.SW.meanP.PSL) 505 and of amplitude (ICP.SW.dP.PSL) 503 is indicated. Here, the relationship between pressure stability level of mean ICP (ICP.SW.meanP.PSL) 505 and of amplitude (ICP.SW.dP.PSL) 503, indicated by 516 differs from the relationship between (ICP.SW.meanP.PSL) 504 and of amplitude (ICP.SW.dP.PSL) 503. The relationships may be outside or inside the third type of thresholds if the relationships deviate from nominal reference relationships. In FIG. 11b, the relationship 517 between different and simultaneous pressure stability level of mean ICP (ICP.SW.meanP.PSL) 511 and of amplitude (ICP.SW.dP.PSL) 507 is indicated. Finally, in FIG. 11c, the relationship 518 between pressure stability level of mean ICP (ICP.SW.meanP.PSL) 513 and of amplitude (ICP.SW.dP.PSL) 512 is indicated. Whether or not the relationship 518 (or 516 and 517) is outside or inside the third type of thresholds can be defined by its deviation from nominal reference relationships. Accordingly, relationships outside the third type of thresholds determine whether baseline pressure indicator (BPi) plots define instability of baseline pressure of the pressure sensor.

It should be noted that while FIGS. 11a-d provide schematic illustrations of various pressure stability levels, examples from real ICP measurements are referred to in this description. For example, the schematic illustrations in FIGS. 11a-c are all shown in FIG. 10d. While the pressure stability level for single wave amplitude (SW.dP.PSL) 406 remained stable and unchanged, the change in pressure stability levels of mean pressure (SW.meanP.PSL) from 410 to 411 illustrates the situation in FIG. 11a, and the change in SW.meanP.PSL from 411 to 412 illustrates the situation in FIG. 11b. If SW.meanP.PSL of 412 remained stable and was compared with 406, it would be illustrative of the situation shown in FIG. 11c.

An overview of some parameters used to calculate pressure stability levels and determine pressure differences between pressure stability levels are summarized in Table 9.

TABLE 9
Some examples of parameters used for various pressures

| | | | Pressure stability | Pressure stability |
|---|---|---|---|---|
| Single | Delta single | Pressure | level pressure | level time |
| wave | wave | stability level | difference | duration |
| SW.meanP | dSW.meanP | SW.meanP.PSL | SW.meanP.PSL.PD | SW.meanP.PSL.TD |
| SW.dP | dSW.dP | SW.dP.PSL | SW.dP.PSL.PD | SW.dP.PSL.TD |
| SW.RTC | dSW.RTC | SW.RTC.PSL | SW.RTC.PSL.PD | SW.RTC.PSL.TD |

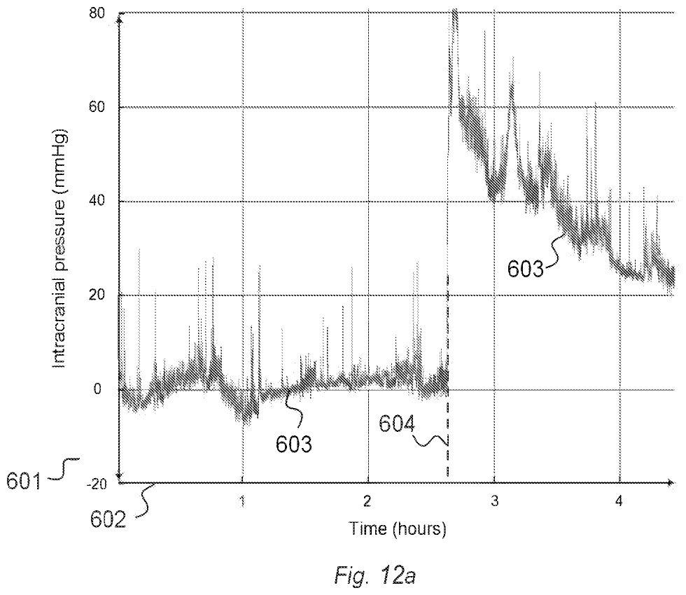

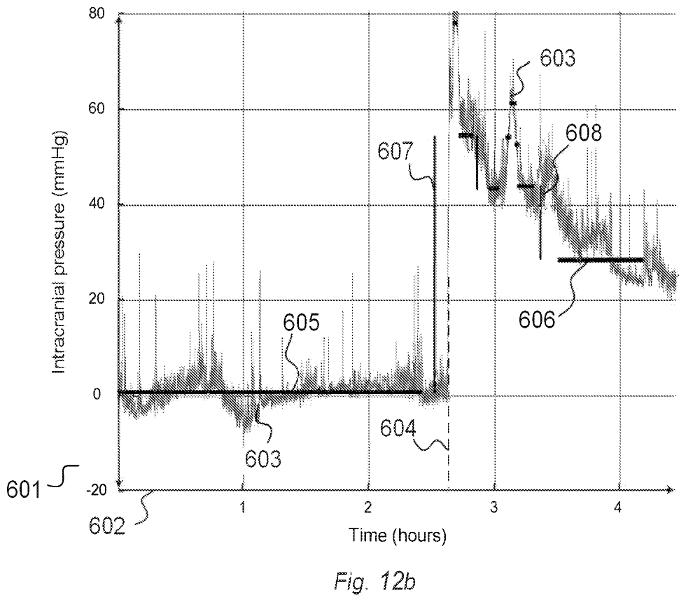

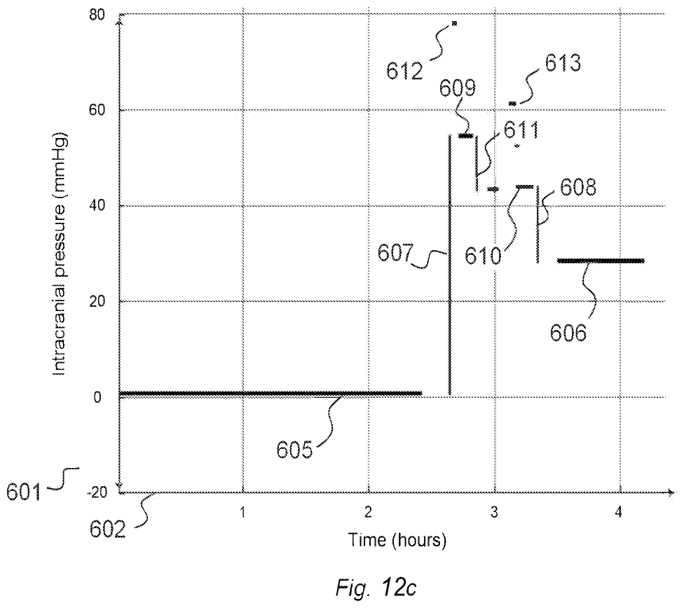

The step of creating baseline pressure indicator (BPi) plots is now further commented on with reference to FIGS. 12a-c, 13a-c, 14a-c, and 16a-g. These figures are retrieved from test software wherein this step was implemented. In particular, it was beneficial to use test software to establish the first, second and third types of thresholds, and to validate the applicability of the steps. Moreover, test software was implemented to compare baseline pressure indicator plots for different types of pressure.

In FIG. 12a the y-axis shows the ICP 601 and the x-axis time 602, and single wave mean pressure (SW.meanP) 603 is plotted as a trend plot. Notably, this example refers to only one single wave related parameter, namely mean ICP (SW.meanP). At one time point 604, a sudden change in mean pressure 603 occurred. Most likely this is because of a sudden shift in the baseline pressure of the pressure sensor. Since the pressure sensor is placed within the brain of the individual, it is impossible to verify with certainty whether or not a baseline shift occurred. In this context, a baseline pressure indicator plot provides information about stability of baseline pressure. In FIG. 12b, the baseline pressure indicator plot 605 is shown. The plot is created from pressure stability levels (SW.meanP.PSL), here illustrated by 605 and 606, and by pressure stability level pressure difference (SW.meanP.PSL.PD), here illustrated by 607 and 608. In FIG. 12c, only the baseline pressure indicator plot is visualized, and incorporates the pressure stability levels (SW.meanP.PSL) 605, 609, 610 and 606, and the pressure stability level pressure differences (SW.meanP.PSL.PD) 607, 608 and 611. In this way, the continuous pressure recording is decomposed into a baseline pressure indicator (or a straight line plot), created from the pressure stability levels 605, 606, 609, 610, and pressure differences between pressure stability levels 607, 608, 611. As already commented on, the pressure stability levels are calculated from a definable number of single pressure waves having delta single pressure wave (dSW.meanP)-related parameters within a first type of selectable thresholds. FIG. 12c shows two additional pressure stability levels 612, 613 that are not included in the baseline pressure indicator plot because time durations of these pressure stability level (SW.meanP.PSL.TD) were outside another set of a first type of selectable thresholds. The baseline pressure indicator plot shown in FIG. 12c may be displayed on an output screen, either alone as in FIG. 12c, or in combination with the trend plot of the single wave parameter as in FIG. 12b. For example, it may be superimposed on the trend plot, demonstrated by different colors. The type or character of presentation represents no limitation with the disclosure.

When monitoring a single pressure wave-related parameter, here exemplified by mean ICP (SW.meanP), the second type of thresholds determine whether the baseline pressure indicator (BPi) plot, defines a presence of baseline pressure instability of a pressure sensor. Pressure differences (SW.meanP.PSL.PD) such as 607, 608, and 611 being outside the second type of thresholds deviate from nominal reference pressure differences determine whether the baseline pressure indicator plot defines baseline pressure instability. The disclosure incorporates alerts if the pressure differences (SW.meanP.PSL.PD) 607, 608 or 611 are outside the thresholds. This is because baseline pressure instability may result in erroneous interpretation of pressure measurements and wrongly patient management. Alerts may be at least one of: a warning color of at least one part of the baseline pressure indicator plot, a warning noise, and a descriptive information.

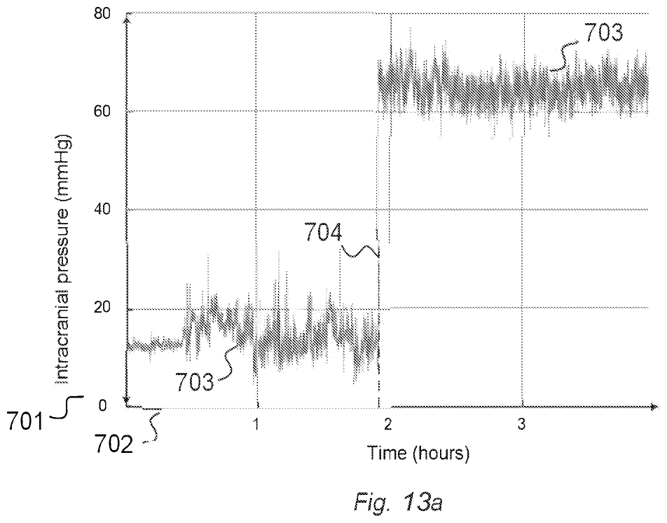

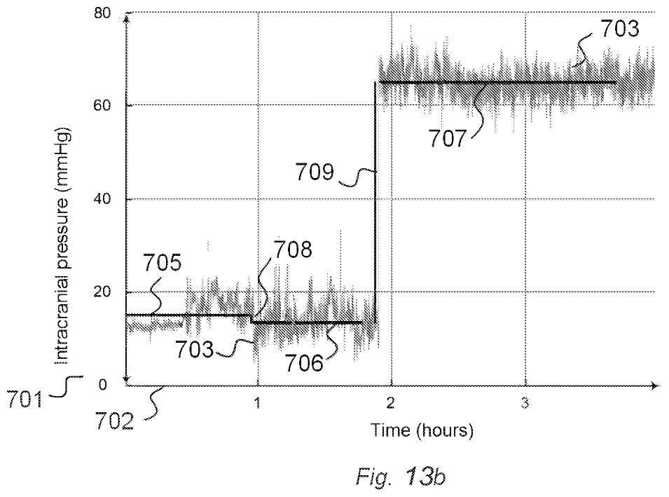

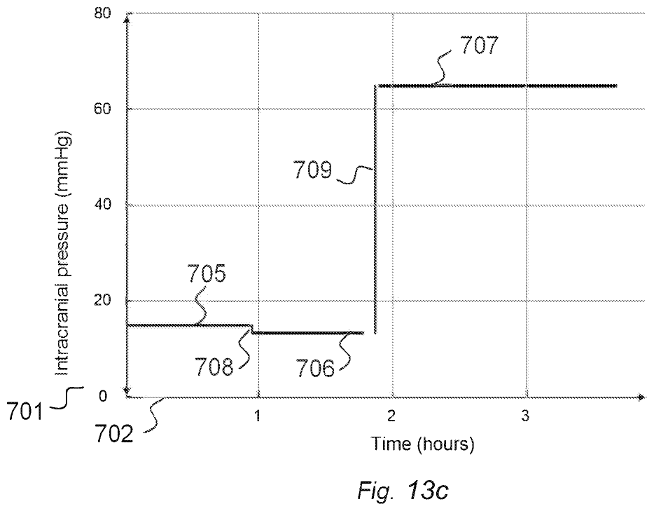

FIGS. 13a-c provide additional examples of how a baseline pressure indicator plot incorporates information about baseline pressure instability. In FIG. 13a, the y axis includes the intracranial pressure scale 701, and the x-axis the time 702. The trend plot of single wave pressure (ICP.SW.meanP) 703 shows a sudden change at time point 704. FIG. 13b from test software shows a baseline pressure indicator plot that is created from the pressure stability levels (ICP.SW.meanP.PSL) 705, 706, 707, and the pressure stability level pressure difference 708, 709. According to empirical observations, the pressure difference (ICP.SW.meanP.PSL.PD) 708 is within second type of thresholds whereas the pressure difference (ICP.SW.meanP.PSL.PD) 709 is outside the second type of thresholds. Therefore, the second type of threshold determines that the baseline pressure indicator plot define baseline pressure instability of the pressure sensor, which can be denoted a baseline pressure error. In FIG. 13c, test software reveals the baseline indicator plot as a straight line only, thereby illustrating how an ICP recording may be presented to provide information not otherwise obtained.

With reference to FIG. 13a, it might be argued that a baseline pressure indicator plot is not needed to indicate a shift in mean pressure. However, the sudden shift in mean pressure shown in FIG. 13a illustrates a very marked pressure change. More commonly, the baseline shifts are less evident. The pressure measurement shown in FIG. 13a is therefore included for illustration purpose.

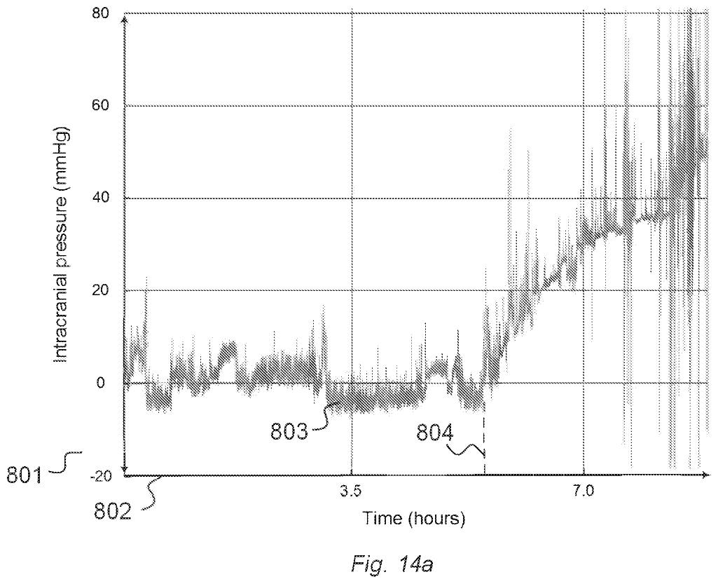

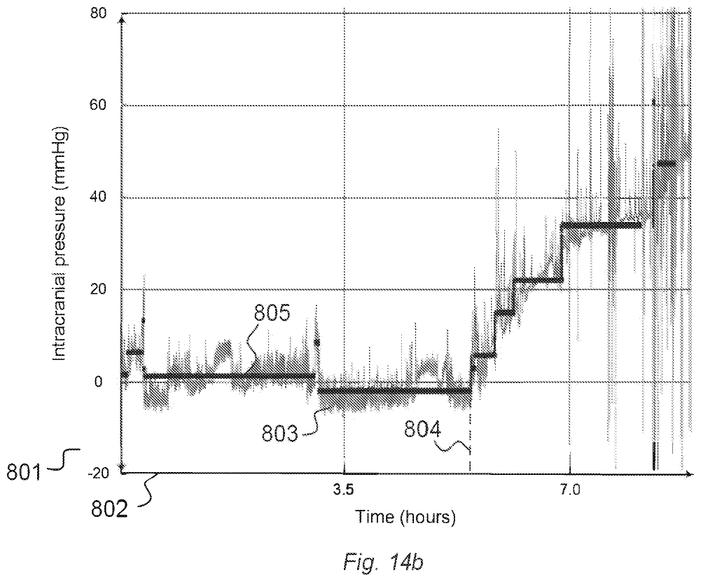

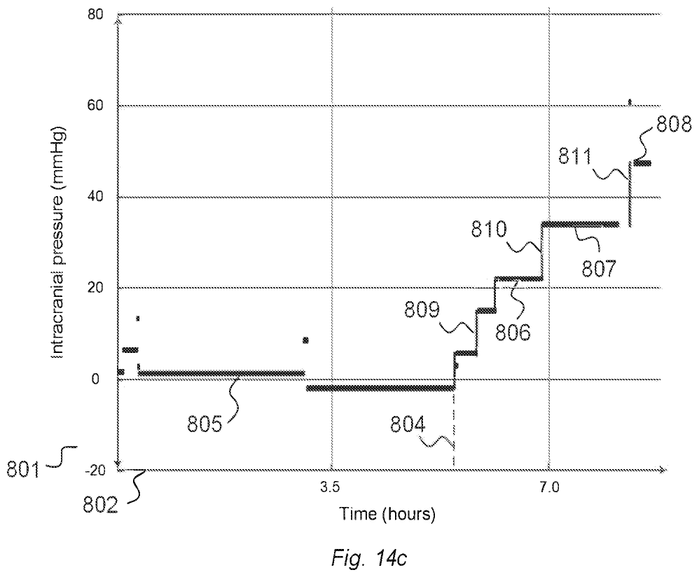

During for example ICP monitoring, it may be questionable whether a change in mean ICP is caused by technical factors or be a result of physiological or pathological changes. One example is given in FIGS. 14a-c, which illustrate the creation of another baseline pressure indicator plot. As shown in FIG. 14a, in a graph with intracranial pressure on the y-scale 801 and time on the x-scale 802, the single wave mean pressure 803 is plotted against time. At time 804 the mean pressure is starting to rise. This rise could either be physiological due to a real increase of ICP, or it might be due to pressure sensor instability and related baseline pressure instability. In FIG. 14b is shown both the mean pressure trend plot 803 and the baseline pressure indicator plot 805. In FIG. 14c, only the baseline pressure indicator plot from test software is presented, which is created by pressure stability levels (ICP.SW.meanP.PSL), some of which are 805, 806, 807 and 808, and the pressure differences (ICP.SW.meanP.PSL.PD) 809, 810 and 811. Whether or not the changes in pressure stability levels 806, 807 and 808 after time 804 refer to physiological or technical causes may be defined by the empirical observations, using the second type of thresholds. In this regard, the second type of thresholds define whether the pressure differences (ICP.SW.meanP.PSL.PD) such as 809, 810 and 811 are inside or outside selectable ranges. In this particular recording, the pressure differences 810 and 811 were outside nominal reference pressure differences. Thereby, the baseline pressure indicator plots defined baseline pressure instability of the pressure sensor.

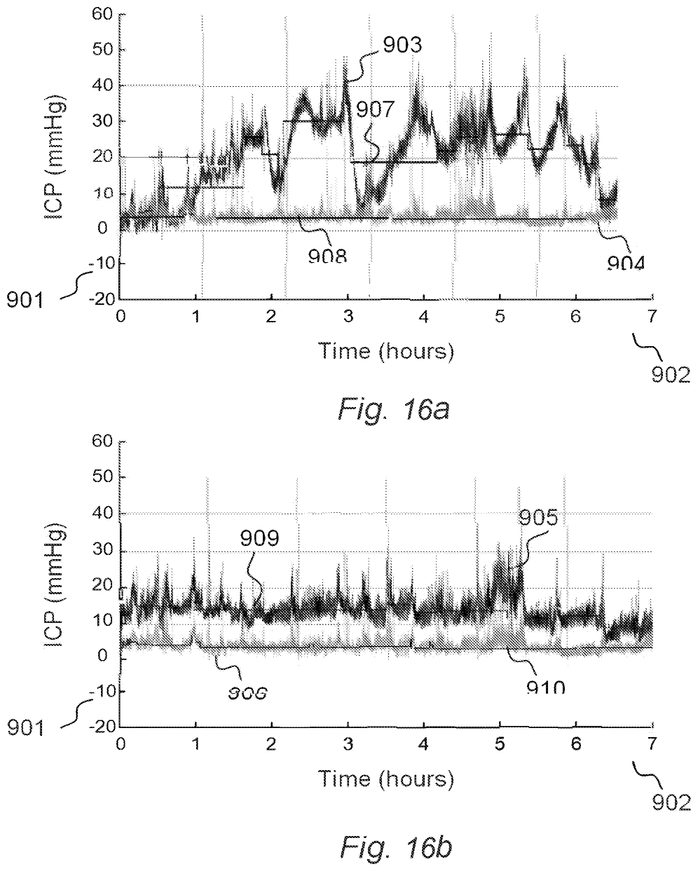

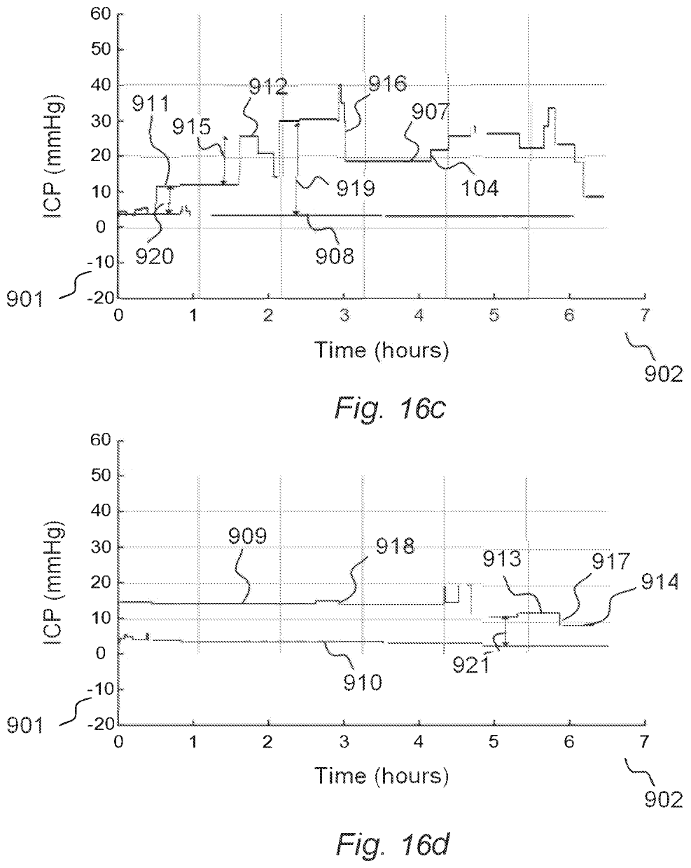

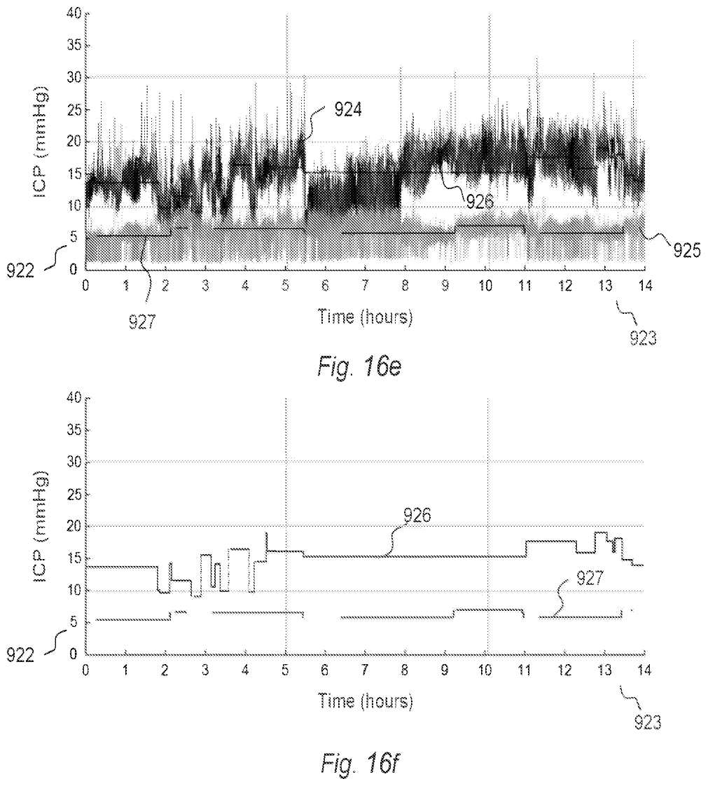

FIGS. 12a-c, 13a-c and 14a-c refer to only one single pressure wave-related parameter to describe in detail the creation of baseline pressure indicator plots. It will be commented on in FIGS. 16a-g that creation of different baseline pressure indicator plots for different single wave (SW.x)-related parameters allows for comparison of pressure stability levels from different single wave parameters. This enables application of the aforementioned third type of threshold. Hence, relationships between e.g., ICP.SW.meanP.PSL and ICP.SW.dP.PSL being outside or inside a third type of selectable set thresholds, reflecting deviations from nominal reference relationships, represents information from baseline pressure indicator plots to define baseline pressure instability of a pressure sensor.

With regard to the first, second and third types of selectable thresholds, the threshold levels depend on several factors, such as type of pressure and type of single wave (SW.x)-related parameters creating the pressure stability levels (SW.x.PSL). For sake of clarity, the present description refers to some selected thresholds for ICP, though the ICP no limitation with the disclosure. The steps described here can be applied to any kind of pressure measured in vivo in humans. Defining the first, second and third types of thresholds were based on utilizing test software and empirical observations. As commented on, for establishment of pressure stability levels, a first type of threshold for ICP.dSW.meanP was used for initial steps, the threshold ranging between 3 to 4 mmHg, 4 to 5 mmHg, 5 to 6 mmHg, and 6 to 7 mmHg. For time duration (SW.meanP.PSL.TD), the thresholds ≥40 dSW or ≥50 dSW observations in the first test experiments were mostly used.

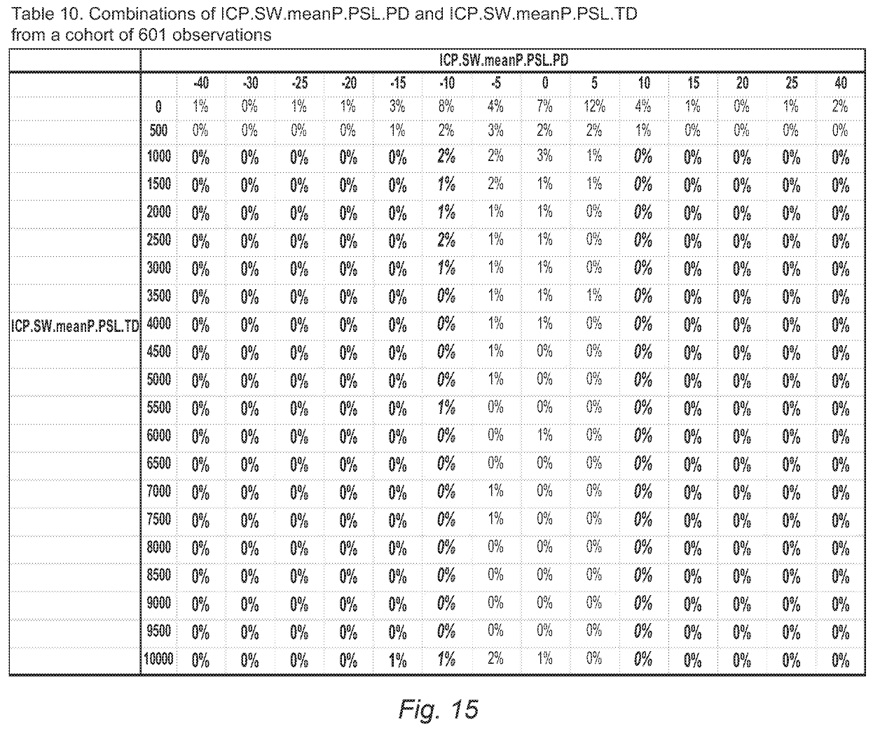

To define the second type of thresholds, several approaches were used, including examinations of pressure differences (SW.meanP.PSL.PD) versus time duration. For example, after a pressure difference (SW.x.PSL.PD), it may be beneficial to identify how long the pressure stability level lasts, or how many observations of delta single wave (dSW.x) parameters are included (SW.x.PSL.TD). Table 10 in FIG. 15 presents the overview of 601 observations of pressure differences (SW.meanP.PSL.PD). While the bold percentages constitute 5% of observations (i.e. corresponding to SW.meanP.PSL.PD≥15/<−15 mmHg and SW.meanP.PSL.TD>1000 single waves). With a heart rate of 60, 1000 single waves correspond to about 16.7 minutes. If also including italic percentages, the observations include 15% of the observations (SW.meanP.PSL.PD≥10/<−10 mmHg and SW.meanP.PSL.TD>1000 single waves). The disclosure is, however, not limited by the exact thresholds for what may be considered pressure differences (SW.meanP.PSL.PD) within or outside the second type of thresholds. The thresholds depend on pressure type and on location from where pressures are measured.

Regarding thresholds for SW.x.PSL.PD and SW.x.PSL.TD, empirical data are determined for specific types of pressures, e.g., for ICP or ABP, the normal combinations of SW.x.PSL.PD and SW.x.PSL.TD are determined using tabular data presentations for large amounts of data. The ICP.SW.x.PSL.PD and ICP.SW.x.PSL.TD combinations outside 95% confidence interval may be defined as being outside threshold.

Moreover, for second type of thresholds, ICP.SW.meanP.PSL.PD>10 mmHg/<−10 mmHg was used in initial experiments to define pressure differences being outside or inside the second type of thresholds, reflecting deviations from nominal reference pressure differences.

To further illustrate another approach for assessment of the second type of thresholds, Table 11 presents some parameters from one individual (TestRecording 13). The entire recording of this individual ICP measurement is presented. A first type of thresholds included: 1) ICP.dSW.meanP <4 mmHg, and 2) ICP.SW.meanP.PSL.TD, ≥50 (i.e. a minimum of 50 dSW.meanP observations was used for establishing a pressure stability level).

TABLE 11
Parameters from TestRecording 13.

| StartIndx | Endindx | ICP.SW.meanP.PSL.TD | ICP.SW.meanP.PSL | ICP.SW.meanP.PSL.PD |
|---|---|---|---|---|
| (=SW_{N}) | (=SW_{N}) | (N) | (mmHg) | (mmHg) |
| 39 | 4365 | 4231 | 16.2 | 0.0 |
| 4388 | 5499 | 1110 | 24.1 | 7.9 |
| 5499 | 7628 | 2102 | 28.9 | 4.7 |
| 7628 | 7976 | 347 | 17.2 | −11.7 |
| 7976 | 8655 | 678 | 30.2 | 13.0 |
| 8655 | 9191 | 535 | 34.3 | 4.1 |
| 9191 | 25402 | 15295 | 15.2 | −19.1 |
| 25474 | 25718 | 206 | 15.8 | 0.6 |
| 25729 | 25849 | 119 | 12.8 | 3.0 |
| 25849 | 33767 | 7553 | 11.3 | −1.5 |
| 33803 | 35117 | 1045 | 12.7 | 1.4 |
| 35161 | 35223 | 61 | 18.9 | 6.2 |
| 35257 | 35332 | 74 | 12.3 | −6.6 |
| 35430 | 35528 | 97 | 7.1 | −5.2 |
| 35545 | 42306 | 6416 | 11.4 | 4.3 |
| 42359 | 54902 | 12003 | 12.5 | 1.2 |
| 54904 | 60166 | 5249 | 18.2 | 5.6 |
| 60215 | 64115 | 3897 | 23.1 | 5.0 |
| 64149 | 68234 | 4040 | 18.4 | −4.8 |
| 68240 | 83941 | 15194 | 46.9 | −1.4 |
| 85120 | 85935 | 1531 | 10.0 | −6.9 |
| 85936 | 87572 | 1551 | 10.5 | 0.5 |

The output of TestRecording13 (Table 11) is useful for assessing the second type of thresholds. In Table 11, there are three instances where ICP.SW.meanP.PSL.PD>10 mmHg, namely for pressure stability levels (ICP.SW.meanP.PSL) with Start/End indices 7628-7976, 7976-8655, and 9191-25402. In some embodiments, second type of thresholds outside these thresholds suggest baseline pressure instability of a pressure sensor. The specific values referred to represent, however, no limitation with the disclosure.

Table 12 presents the single wave parameter mean ICP (SW.meanP) from the ICP recording of another individual (TestRecording 44). As can be seen, the pressure recording is divided into pressure stability levels (ICP.SW.meanP.PSL). The time duration is given by the number of dSW.meanP observations in the column ICP.SW.meanP.PSL.TD, which includes single waves from StartIndex to EndIndex. The average of mean ICP for each pressure stability level is presented in column ICP.SW.meanP.PSL. Moreover, the pressure difference (ICP.SW.meanP.PSL.PD) from one pressure stability level to the next is given in the column ICP.SW.meanP.PSL.PD.

TABLE 12
Parameters from TestRecording 44.

| StartIndx | Endindx | ICP.SW.meanP.PSL.TD | ICP.SW.meanP.PSL | ICP.SW.meanP.PSL.PD |
|---|---|---|---|---|
| (= SW_{N}) | (= SW_{N}) | (N) | (mmHg) | (mmHg) |
| 141 | 2455 | 1923 | 10.3 | 0.0 |
| 2711 | 3309 | 199 | 3.1 | −7.2 |
| 3515 | 8682 | 4640 | 12.2 | 9.0 |
| 9000 | 16511 | 4325 | 10.1 | −2.1 |
| 16756 | 24292 | 6620 | 16.7 | 6.6 |
| 24874 | 30784 | 5804 | 15.2 | −1.5 |
| 30784 | 30903 | 118 | 21.9 | 6.7 |
| 30934 | 31108 | 173 | 27.8 | 6.0 |
| 31110 | 31177 | 66 | 36.3 | 8.5 |
| 31251 | 31643 | 204 | 32.1 | −4.2 |
| 31573 | 31714 | 137 | 28.4 | −3.7 |
| 31770 | 37195 | 5063 | 13.7 | −14.7 |
| 37199 | 37599 | 366 | 22.8 | 9.1 |
| 37599 | 37654 | 54 | 28.7 | 5.9 |
| 37664 | 37826 | 127 | 21.9 | −6.8 |
| 37866 | 38088 | 202 | 28.5 | 6.6 |
| 38088 | 38172 | 83 | 36.5 | 8.0 |
| 38328 | 38405 | 76 | 23.6 | −13.0 |
| 38407 | 38794 | 386 | 14.6 | −9.0 |
| 38794 | 38994 | 199 | 26.8 | 12.2 |
| 38994 | 39417 | 401 | 30.1 | 3.3 |
| 39418 | 40167 | 692 | 15.4 | −14.7 |
| 40167 | 40658 | 481 | 26.5 | 11.0 |
| 40663 | 41007 | 343 | 14.6 | −11.9 |
| 41007 | 41154 | 146 | 19.5 | 5.0 |
| 41162 | 47160 | 5744 | 13.7 | −5.9 |
| 47160 | 47231 | 70 | 28.4 | 14.7 |
| 47231 | 47512 | 270 | 18.8 | −9.6 |
| 47519 | 49140 | 1284 | 10.4 | −8.4 |
| 49145 | 49726 | 246 | 2.9 | −7.5 |

The analysis output of TestRecording 44 (Table 12) also illustrates how the second type of thresholds can be assessed. In Table 12, there are seven instances where ICP.SW.meanP.PSL.PD>10 mmHg, namely for pressure stability levels (ICP.SW.meanP.PSL) with Start/End indices 31770-37195, 38328-38405, 38794-38994, 39418-40167, 40167-40658, 40663-41007, and 47160-47231. The significance of a ICP.SW.meanP.PSL.PD>10 mmHg depends on its duration (ICP.SW.meanP.PSL.TD), which also define whether the second type of thresholds are inside or outside defined ranges to indicate pressure sensor instability. The data presented here highlight that thresholds need to be defined from empirical observations. A threshold of ICP.SW.meanP.PSL.PD>10 mmHg represents no limitation with the disclosure.

Also regarding the third type of thresholds, empirical observations were needed to define which thresholds that are inside or outside defined ranges reflecting deviations from nominal reference thresholds. For this purpose, it may be useful to assess a large amount of observations, utilizing different approaches. In Table 13, the distribution of pressure stability levels for single wave amplitude (ICP.SW.dP.PSL) versus mean pressure (ICP.SW.meanP.PSL) is presented. This matrix was based on pressure recordings incorporating 1,307,520 single waves. The bold percentages constitute 39% of observations. These cells can be indicated as abnormal and indicative of instability of baseline pressure.

TABLE 13
Distribution of pressure stability levels for single wave amplitude
(ICP.SW.dP.PSL) versus mean pressure (ICP.SW.meanP.PSL), based on a cohort of
1,307,520 single waves.

| | | ICP.SW.dP.PSL (mmHg) | | | | | | | | | |
|---|---|---|---|---|---|---|---|---|---|---|---|
| | | 1 | 2 | 3 | 4 | 5 | 6 | 7 | 8 | 9 | 10 |
| ICP.SW.meanP.PSL | −15 | 0% | 0% | 0% | 1% | 0% | 0% | 0% | 0% | 0% | 0% |
| (mmHg) | −10 | 0% | 0% | 0% | 1% | 0% | 0% | 0% | 0% | 0% | 0% |
| | −5 | 0% | 0% | 0% | 2% | 0% | 0% | 2% | 0% | 0% | 0% |
| | 0 | 0% | 0% | 1% | 4% | 2% | 1% | 11% | 0% | 2% | 0% |
| | 5 | 2% | 0% | 3% | 4% | 0% | 3% | 5% | 1% | 2% | 0% |
| | 10 | 0% | 0% | 6% | 3% | 0% | 0% | 1% | 0% | 3% | 0% |
| | 15 | 0% | 3% | 8% | 2% | 0% | 0% | 0% | 0% | 0% | 0% |
| | 20 | 0% | 1% | 1% | 1% | 0% | 0% | 0% | 0% | 0% | 0% |
| | 25 | 0% | 0% | 1% | 1% | 0% | 0% | 0% | 0% | 0% | 0% |
| | 30 | 0% | 0% | 0% | 3% | 0% | 0% | 0% | 0% | 0% | 0% |
| | 35 | 0% | 0% | 0% | 2% | 0% | 0% | 0% | 0% | 0% | 0% |
| | 40 | 1% | 0% | 0% | 11% | 0% | 0% | 0% | 0% | 0% | 0% |

Information derived from Table 13 is that pressure stability level (ICP.SW.dP.PSL) 3-4 mmHg combined with ICP.SW.meanP.PSL <−5 mmHg or ≥25 mmHg is extremely rare.

The foregoing paragraphs have discussed in detail creation of baseline pressure indicator plots applying a first type of thresholds, and define whether baseline pressure indicator plots indicate pressure sensor instability and related baseline pressure instability applying a second and third type of thresholds. In FIGS. 16a-g the methodology is applied to ICP recordings in humans to further illustrate its applicability.

With reference to the ICP trend plots shown in FIGS. 7a-b, it is now illustrated in FIGS. 16a-b how the disclosure may be applied to assess pressure sensor instability. The simultaneous ICP recordings are useful for showing how the disclosure may be used. For more details, see description for FIGS. 7a-b. In short, the y-axis 901 shows ICP in mmHg and the x-axis 902 the time in hours. In FIG. 16a (see also FIG. 7a) is shown the trend plots of mean ICP 903 and MWA 904, measured by the Camino ICP sensor. In FIG. 16b (see also FIG. 7b) is shown the trend plots of mean ICP 905 and MWA 906, measured by the Codman ICP sensor, placed nearby the Camino ICP sensor. The test software was applied to the recordings. The first type of thresholds were as follows: 1) dSW.meanP <4 mmHg for 50 consecutive dSW.meanP observations, 2) dSW.dP<1 mmHg for 25 consecutive dSW.dP observations. 3) Nearby baseline pressure stability levels (SW.meanP.PSL) with dSW.meanP <4 mmHg were merged. The resulting baseline pressure indicator (BPi) plots for mean ICP 907 and MWA 908 of the Camino ICP are presented in FIG. 16a, and the baseline pressure indicator (BPi) plots for mean ICP 909 and MWA 910 of the Codman ICP are presented in FIG. 16b. The baseline pressure indicator plots superimposed on trend plots of ICP scores may be somewhat hard to visualize. Therefore, the test software provides the option to subtract trend plots for better visualization. This is further shown in FIGS. 16c-d.

With reference to FIGS. 16c-d, the baseline indicator plot for mean ICP 907 of the Camino ICP sensor differs markedly from BPi plot of mean ICP 909 of Codman ICP. As can be seen for mean ICP of Camino ICP (FIG. 9c), the BPi plot consists of several baseline stability levels (SW.meanP.PSL), i.e. horizontal parts of BPi plot, as indicated in 911 and 912. Each pressure stability level (SW.meanP.PSL) has different time duration (SW.meanP.PSL.TD). Likewise, for the Codman ICP sensor (FIG. 16d), the BPi plot of mean ICP has several pressure stability levels of variable duration, as exemplified in 913 and 914. While the BPi plots of MWA are comparable for the Camino ICP 908 (FIG. 16c) and Codman ICP 910 (FIG. 16d), the BPi plots of mean ICP 907, 909 differs extensively. When assessing which of the ICP sensors that present with baseline pressure instability and related baseline pressure instability, the second and third type of thresholds of this disclosure are applied.

The second type of thresholds assess differences in pressure stability levels calculated from the same type of single pressure wave (SW.x)-related parameters, such as BPi for mean ICP of Camino pressure sensor 907 (FIG. 16c). One set of the second type of threshold define that pressure differences between pressure stability levels (SW.x.PSL.PD) of certain time durations should be within ranges, reflecting nominal reference pressure differences. For example, the SW.meanP.PSL.PD 915 between pressure stability levels 911 and 912 are >10 mmHg, which is outside the second type of thresholds for this particular pressure and single wave parameter. The SW.meanP.PSL.PD 916 is another example of a SW.meanP.PSL.PD score being outside a second type of threshold. On the other hand, the SW.meanP.PSL.PD of Codman sensor 917 (FIG. 16d) is <5 mmHg and thereby being inside a second type of threshold. Yet another example of SW.meanP.PSL.PD being inside the second type of threshold is shown in 918. The exact levels for the second type of thresholds depend on several factors such as the single wave parameters; the levels referred to here represent no limitation of the disclosure. Hence, for SW.dP.PSL.PD of Camino MWA 908 (FIG. 16c) and Codman MWA 910 (FIG. 16d), no SW.dP.PSL.PD observations were outside the second type of thresholds, thus not reflecting deviations from nominal reference pressure differences for this particular single wave (SW.x)-related parameter.

Moreover, baseline pressure indicator plots may also define baseline pressure instability of a pressure sensor applying a third type of thresholds. The third type of thresholds refer to different types of single pressure wave (SW.x)-related parameters of a baseline pressure indicator plot. With reference to BPi plots of mean ICP 907 and MWA 908 of Camino sensor (FIG. 16c), the third type of criteria define how BPi plots of mean ICP 907 and MWA 908 relate. Examples of the third type of thresholds are given. The relationship between different and simultaneous pressure stability levels, here between SW.meanP.PSL and SW.dP.PSL, may be the dividend SW.meanP.PSL/SW.meanP.PSL. The relationship should not be outside or inside the third type of selectable set thresholds, reflecting deviations from nominal reference relationships. One example of a relationship (here dividend) outside the third type of thresholds is given in 919, while another example is given in 920, which is inside the third type of thresholds (FIG. 16c). The relationship SW.meanP.PSL/SW.meanP.PSL of FIG. 16d was inside the third type of thresholds, as indicated in 921. Other third type of thresholds relate to comparisons of change in pressure stability levels, for example SW.meanP.PSL.PD 915 versus SW.dP.PSL.PD 908. Accordingly, a relationship between different and simultaneous pressure stability levels (n−1; n) (here exemplified by the dividend SW.meanP.PSL.PD/SW.dP.PSL.PD) should be outside the third type of thresholds for a baseline pressure indicator plot to define instability of baseline pressure of a pressure sensor.

Baseline pressure indicator (BPi) plots with parts of the plot or the entire plot being outside the second or third types of thresholds may be presented with different warning colors, warning noise or descriptive information to alert about deviation from normative values. In addition, data presentations may be given. Table 14a provides quantitative data of the BPi plot of mean ICP of the Camino ICP sensor 907 (FIG. 16c). Table 14b provides quantitative data of the BPi plot of MWA of the Camino ICP sensor 908 (FIG. 16d).

TABLE 14A

| SW.MeanP.PSL | SW.MeanP.PSL.TD | | | |
|---|---|---|---|---|
| (mmHg) | (min) | Start Indx | End Indx | SWCount |
| 3.5 | 27.9 | 0 | 2338 | 2070 |
| 8.0 | 0.6 | 2693 | 2742 | 48 |
| 3.8 | 14.9 | 2757 | 3943 | 1104 |
| 12.5 | 21.8 | 3943 | 6004 | 1613 |
| 19.2 | 1.4 | 6031 | 6136 | 104 |
| 16.9 | 2.6 | 6157 | 6355 | 194 |
| 19.4 | 3.0 | 6407 | 6632 | 222 |
| 24.0 | 0.8 | 6639 | 6785 | 60 |
| 19.5 | 4.6 | 6875 | 7272 | 341 |
| 28.6 | 0.4 | 7419 | 7450 | 30 |
| 25.9 | 8.9 | 7497 | 8596 | 663 |
| 30.3 | 2.3 | 8643 | 8812 | 168 |
| 25.2 | 1.2 | 8817 | 8906 | 88 |
| 19.7 | 1.8 | 8912 | 9049 | 136 |
| 14.8 | 5.2 | 9049 | 9777 | 386 |
| 18.9 | 0.9 | 9835 | 9902 | 66 |
| 23.7 | 5.9 | 9902 | 10342 | 439 |
| 33.9 | 15.1 | 10342 | 11489 | 1119 |
| 30.1 | 22.7 | 11489 | 13378 | 1683 |
| 28.6 | 0.9 | 13731 | 13806 | 64 |
| 24.1 | 1.0 | 13837 | 13912 | 74 |
| 16.5 | 3.1 | 13912 | 14140 | 227 |
| 9.9 | 19.4 | 14140 | 15948 | 1434 |
| 17.7 | 4.3 | 15948 | 16287 | 321 |
| 25.7 | 22.7 | 16288 | 18742 | 1679 |
| 22.6 | 12.8 | 18742 | 20007 | 945 |
| 27.6 | 1.1 | 20172 | 20508 | 78 |
| 23.9 | 11.1 | 21247 | 23439 | 824 |
| 31.3 | 1.3 | 23881 | 23980 | 98 |
| 35.5 | 0.8 | 24159 | 24319 | 62 |
| 29.9 | 0.4 | 24347 | 24381 | 33 |
| 25.5 | 2.7 | 24381 | 24585 | 203 |
| 23.6 | 18.6 | 24612 | 26347 | 1380 |
| 36.2 | 0.6 | 26380 | 26424 | 43 |
| 22.7 | 12.9 | 26692 | 27806 | 959 |
| 17.0 | 3.9 | 27836 | 28434 | 291 |
| 8.6 | 6.7 | 28674 | 29517 | 500 |

TABLE 14B

| SW.dP.PSL | SW.dP.PSL.TD | | | |
|---|---|---|---|---|
| (mmHg) | (min) | Start Indx | End Indx | Count |
| 3.2 | 0.5 | 0 | 133 | 130 |
| 4.4 | 0.9 | 133 | 536 | 226 |
| 5.1 | 0.2 | 556 | 595 | 38 |
| 3.6 | 0.4 | 998 | 1106 | 106 |
| 4.5 | 0.2 | 1106 | 1164 | 45 |
| 5.0 | 0.6 | 1164 | 1514 | 142 |
| 5.5 | 0.7 | 1545 | 1879 | 170 |
| 8.0 | 0.1 | 1977 | 2001 | 23 |
| 5.5 | 0.1 | 2178 | 2199 | 20 |
| 3.9 | 3.0 | 2218 | 3872 | 737 |
| 4.6 | 0.3 | 3874 | 3976 | 71 |
| 6.1 | 0.1 | 4201 | 4228 | 26 |
| 3.6 | 4.6 | 4229 | 11503 | 1148 |
| 3.6 | 4.7 | 11658 | 15834 | 1159 |
| 3.2 | 0.1 | 15883 | 15904 | 20 |
| 4.7 | 0.3 | 15974 | 16039 | 63 |
| 3.4 | 7.1 | 16224 | 29360 | 1769 |

Tables 15a and 15b provide quantitative data of the BPi plot of mean ICP 909 and MWA 910 of the Codman ICP sensor (FIG. 16c).

TABLE 15A

| SW.MeanP.PSL | SW.MeanP.PSL.TD | | | |
|---|---|---|---|---|
| (mmHg) | (min) | Start Indx | End Indx | SWCount |
| 13.1 | 10.4 | 0 | 708 | 705 |
| 18.9 | 2.6 | 708 | 946 | 175 |
| 15.0 | 15.7 | 995 | 2215 | 1064 |
| 12.0 | 2.4 | 2215 | 2378 | 162 |
| 15.0 | 17.8 | 2693 | 3995 | 1207 |
| 19.8 | 2.1 | 3995 | 4139 | 143 |
| 23.3 | 0.5 | 4139 | 4171 | 31 |
| 14.0 | 128.4 | 4203 | 14879 | 8693 |
| 15.5 | 18.6 | 15017 | 16453 | 1258 |
| 13.2 | 16.7 | 16453 | 17693 | 1128 |
| 13.8 | 26.5 | 18161 | 21656 | 1793 |
| 11.8 | 16.8 | 22677 | 23840 | 1135 |
| 8.8 | 1.2 | 24151 | 24231 | 79 |
| 15.0 | 0.5 | 24453 | 24486 | 32 |
| 12.4 | 25.1 | 24632 | 26881 | 1697 |
| 9.1 | 34.0 | 27118 | 29995 | 2304 |

TABLE 15B

| SW.dP.PSL | SW.dP.PSL.TD | | | |
|---|---|---|---|---|
| (mmHg) | (min) | Start Indx | End Indx | Count |
| 3.6 | 0.6 | 10 | 191 | 126 |
| 5.3 | 0.1 | 192 | 224 | 31 |
| 4.3 | 0.4 | 276 | 410 | 95 |
| 6.1 | 0.1 | 429 | 450 | 20 |
| 4.5 | 0.4 | 450 | 564 | 83 |
| 6.0 | 0.1 | 585 | 611 | 25 |
| 5.2 | 0.3 | 643 | 988 | 57 |
| 4.6 | 1.4 | 1037 | 1657 | 296 |
| 4.6 | 0.1 | 1792 | 1825 | 32 |
| 4.3 | 3.6 | 1896 | 3924 | 792 |
| 4.2 | 0.1 | 3935 | 3963 | 27 |
| 5.3 | 0.1 | 4002 | 4026 | 23 |
| 7.1 | 0.1 | 4028 | 4051 | 22 |
| 5.1 | 0.3 | 4286 | 4495 | 68 |
| 3.8 | 5.3 | 4511 | 10592 | 1159 |
| 4.6 | 0.1 | 10805 | 10829 | 23 |
| 3.7 | 3.2 | 11219 | 13399 | 689 |
| 4.0 | 2.5 | 13434 | 16249 | 554 |
| 5.9 | 0.1 | 16278 | 16302 | 23 |
| 3.4 | 1.4 | 16453 | 17307 | 304 |
| 5.2 | 0.4 | 17338 | 17360 | 21 |
| 4.3 | 0.3 | 17387 | 17500 | 61 |
| 3.3 | 5.9 | 17596 | 24565 | 1292 |
| 3.7 | 4.0 | 25818 | 29835 | 873 |

Applying the second and third types of thresholds, it becomes evident that the BPi plot of the Camino sensor (FIG. 16c) is outside thresholds, indicating pressure sensor instability and related baseline pressure instability.

Usually, ICP is measured from only one ICP sensor. One example of using the test-software is given in FIGS. 16e-f, which is now commented on. The y axis 922 shows the ICP in mmHg and the x-axis 923 the time in hours. FIG. 16e shows trend plots of mean ICP 924 and MWA 925. Furthermore, the BPi of mean ICP 926 is presented, as well as the BPi of MWA 927. Since the BPi shown together with the trend plots may be difficult to visualize, in FIG. 16f only the BPi plots are shown. For creation of pressure stability levels, the first type of thresholds were: dSW.meanP<3 mmHg; SW.meanP.PSL.TD>50 single waves; dSW.dP<1 mmHg; SW.meanP.PSL.TD>25 single waves. The second type of thresholds were: SW.meanP.PSL.PD<10 mmHg, and SW.dP.PSL.PD<5 mmHg. The third type of threshold was SW.meanP.PSL/SW.dP.PSL >4 to be out of range. With these criteria, the baseline pressure indicator plots of mean ICP 926 or MWA 927 did not define instability of baseline pressure of the ICP sensor.

The quantitative data concerning mean ICP for the recording including is presented in Table 16.

TABLE 16
showing different levels of SW.MeanP.PSL.

| SW.MeanP.PSL | SW.MeanP.PSL.TD | | | |
|---|---|---|---|---|
| (mmHg) | (min) | Start Indx | End Indx | SWCount |
| 6.9 | 1.2 | 15 | 98 | 82 |
| 13.7 | 93.2 | 138 | 7297 | 6318 |
| 10.2 | 2.2 | 7297 | 7508 | 147 |
| 9.8 | 10.6 | 7537 | 8488 | 721 |
| 14.3 | 1.8 | 8534 | 8657 | 121 |
| 11.7 | 20.3 | 8791 | 10584 | 1378 |
| 9.2 | 6.9 | 10956 | 11566 | 465 |
| 15.5 | 9.2 | 11607 | 12557 | 626 |
| 10.7 | 2.8 | 12723 | 12921 | 187 |
| 14.2 | 3.7 | 12996 | 13459 | 248 |
| 10.0 | 11.1 | 13491 | 14330 | 754 |
| 16.5 | 19.7 | 14602 | 16371 | 1334 |
| 9.9 | 4.6 | 16389 | 16838 | 309 |
| 14.5 | 12.4 | 17042 | 18039 | 841 |
| 18.9 | 0.6 | 18048 | 18092 | 43 |
| 16.2 | 9.9 | 18230 | 18987 | 673 |
| 16.1 | 28.4 | 19128 | 21705 | 1923 |
| 15.3 | 270.7 | 21836 | 43757 | 18346 |
| 17.7 | 64.5 | 43807 | 48764 | 4370 |
| 15.9 | 24.6 | 48764 | 50611 | 1665 |
| 19.0 | 15.3 | 50635 | 51775 | 1034 |
| 17.7 | 8.6 | 51775 | 52361 | 581 |
| 16.0 | 2.8 | 52361 | 52549 | 187 |
| 18.1 | 10.1 | 52549 | 53259 | 687 |
| 14.9 | 13.9 | 53263 | 54241 | 940 |
| 14.0 | 13.4 | 54242 | 55320 | 906 |

Another example from the test software is presented in FIG. 16g. This further shows comparisons of baseline pressure indicator plots of the single wave (SW.x)-related parameters mean ICP and MWA. Intracranial pressure is shown on the y-axis 928 and time on the x axis 929. The mean pressure (SW.meanP) 930 is plotted against time 929, illustrating the trend plot of SW.meanP 930. In addition, the pressure stability level of mean ICP (SW.meanP.PSL) 931 is visualized with the same time reference 929. In addition, the single wave amplitude (SW.dP) 932 is plotted against time 929, including pressure stability level of single wave amplitude (SW.dP.PSL) 933. It may be noted that the pressure stability levels differ markedly for the single wave parameters mean pressure (SW.meanP) and amplitude (SW.dP). The pressure stability level pressure difference 934 may be caused by several factors, such as different position of the patient being monitored (position-dependent physiological changes in mean pressure) and instability of the pressure sensor (technical flaws of the pressure sensor). To assess presence of baseline pressure instability, empirical data regarding thresholds for SW.x.PSL, SW.x.PSL.PD and SWx.PSL.TD are needed. For example, from empirical data it may be determined whether the differences in pressures between pressure stability levels (SW.meanP.PSL.PD) 934 are outside or inside the second type of thresholds. In this particular case, the change in pressure stability level for mean ICP (SW.meanP.PSL.PD) 934 was outside the second type of thresholds, i.e. the baseline pressure indicator plot of mean ICP defines instability of baseline pressure of the pressure sensor. With regard to the third type of thresholds, the relationship between the different and simultaneous pressure stability levels of mean ICP (SW.meanP.PSL) 931 and of ICP wave amplitude (SW.dP.PSL) 933 were inside a third type of selectable set thresholds, not reflecting deviation from nominal reference relationships. For FIG. 16g, the BPi plot of mean ICP, not the BPI plot of ICP wave amplitude, defined instability of baseline pressure of the pressure sensor since only pressure differences between different pressure stability of mean ICP (SW.meanP.PSL) were outside the second type of selectable set thresholds.

As already commented on, the term baseline pressure indicator plot may be used because definite proof for the presence of baseline pressure alterations during in vivo pressure measurements might not be obtained. The information from parameters related to the second and third types of thresholds indicate, not prove, whether baseline pressure indicator plots define baseline pressure instability. Accordingly, the disclosure does not claim proof, but rather indication, of baseline pressure instability.

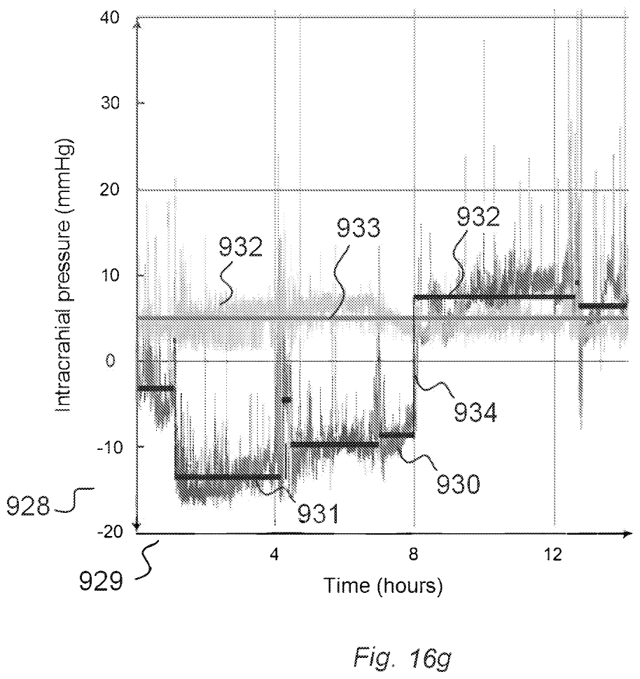

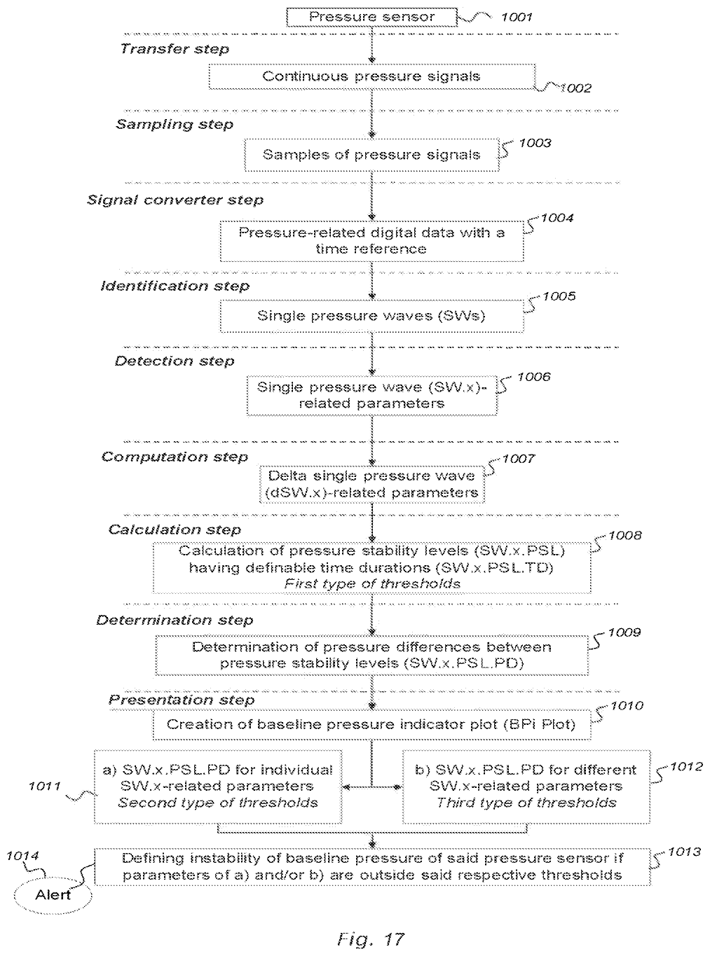

Aspects of the disclosure provide means for determining baseline pressure indicator plots, and issuance of an alert depending on magnitude and duration of baseline pressure instability. FIG. 17 illustrates a method for assessing baseline pressure instability of a pressure sensor applied for sampling of continuous pressure signals, according to embodiments of the present disclosure.

FIG. 17 illustrates aspects of the disclosure, including a method for assessing stability of baseline pressure of a pressure sensor 1001. The method may include receiving, from the pressure sensor 1001, continuous pressure signals 1002 measured from inside a human body or body cavity and sampling the continuous pressure signals to sampled continuous pressure signals. Samples of the pressure signals 1003 from the sensor may be obtained at specific intervals, and may be converted into pressure-related digital data with a time reference 1004.

The method further includes:

identifying single pressure waves 1005 related to cardiac beat-induced pressure waves from the digital data,

detecting single pressure wave (SW.x)-related parameters 1006 selectable from one or more of single wave mean pressure (SW.meanP) and single wave amplitude (SW.dP), and

computing one or more delta single pressure wave (dSW.x)-related parameters 1007, representing differences in single pressure wave (dSW.x)-related parameters selectable from one or more of change in mean pressure (dSW.meanP) and change in amplitude (dSW.dP) between a consecutive number of single pressure waves (n−1; n),

wherein calculation of pressure stability levels (SW.x.PSL) 1008 of the single pressure wave (SW.x)-related parameters 1006 is created from consecutive single pressure waves having any one of the delta single pressure wave (dSW.x)-related parameters dSW.meanP and dSW.dP 1007 within a first set of thresholds, the first set of thresholds referring to defined pressure ranges of any one of the parameters dSW.meanP and dSW.dP 1007, and wherein a pressure stability level 1008 refers to an average of any one of the single pressure wave (SW.x)-related parameters SW.meanP and SW.dP 1006,

wherein a determination is made of pressure differences between different pressure stability levels (n−1; n) (SW.x.PSL.PD) 1009,

the pressure stability levels (SW.x.PSL) 1008 having definable time durations (SW.x.PSL.TD) relating to a time duration of the pressure stability levels (SW.x.PSL),

and the pressure stability levels 1008 of variable time durations (SW.x.PSL.TD) and with beginning pressure differences and ending pressure differences (SW.x.PSL.PD) 1009 for each pressure stability level (SW.x.PSL) together creating a baseline pressure indicator (BPi) plot 1010, the beginning pressure difference being defined as a difference between a present pressure stability level pressure stability level and a previous pressure stability level and the ending pressure difference being defined as a difference between a present pressure stability level and a next pressure stability level,

the BPi plot providing information about stability of baseline pressure of the pressure sensor and being a function of at least one of:

a) combinations of the pressure differences between different pressure stability levels (SW.x.PSL) 1008, calculated from a same type of single pressure wave (SW.x)-related parameters, the pressure differences being outside or inside a second set of thresholds 1011, reflecting deviations from nominal reference pressure differences, and

b) relationships between different and simultaneous pressure stability levels (n−1; n) calculated from different types of single pressure wave (SW.x)-related parameters 1006, the relationships being outside or inside a third set of thresholds 1012, reflecting deviations from nominal reference relationships, and

wherein parameters of a) and/or b) outside the second set and/or the third set of thresholds thereby define instability of baseline pressure of the pressure sensor 1013.

A human body cavity may refer to a cranio-spinal cavity providing for measurement of intracranial pressure. It may also refer to a blood-vessel compartment providing for measurement of arterial blood pressure. Regarding intracranial pressure, it may be measured in a variety of ways: Most commonly, ICP is measured by implanting a wired-based sensor 1001 into the brain parenchyma. Another strategy is measuring cerebrospinal fluid pressure via a pressure sensor 1001 connected to a catheter placed in one of the CSF cavities of the intracranial compartment or via spinal puncture to the thecal sac. A less invasive method is epidural placement of a pressure sensor 1001, i.e. inside the scull but outside the dura mater of the brain. Commonly, specific pressure sensors 1001 are used for measuring of intracranial pressure (ICP), while arterial blood pressure (ABP) usually is measured via a fluid-based sensor 1001. Hence, a fluid-filled catheter is placed within the blood vessel, and an external pressure sensor 1001 is measuring the pressure within the vessel via the fluid filled catheter in communication with the sensor 1001.

The pressure sensor is configured for measuring of intracranial pressure (ICP) or arterial blood pressure (ABP) signals.

Pressure sensors 1001 may be implanted for a shortened or temporary time period or for an extended period of weeks or months. Wired-based sensors 1001 are implanted for a temporary period since these sensors usually penetrate the skin, which may represent a risk for infection. Other pressure sensors 1001 may be implanted for a longer period.

For example, miniature sensors 1001 may be implanted within the blood vessel or the intracranial compartment. Pressure signals 1002 obtainable by the pressure sensor 1001 are wirelessly transferred when the sensor 1001 is implanted for the extended period of weeks or months. In this disclosure, the term “transfer” is synonymous with “communicate.” In some embodiments, a miniature sensor 1001 having the ability for providing continuous pressure signals 1002 via wire-less means is located epidural. A receiver external to the head may retrieve the pressure signals. Such a miniature sensor 1001 may be implanted on a permanent basis.

In some embodiments, a pressure sensor 1001 is connected to a catheter for drainage of CSF, enabling measurement of CSF pressure. The catheter may be part of a shunt system for drainage of CSF. An external receiver may retrieve the continuous pressure signals 1002.

The pressure-related digital data with a time reference 1004 are obtained via a signal converter step, followed by identification of single pressure waves 1005. A variety of single pressure wave parameters 1006 may be detected.

The mean pressure represents a static pressure being mean single wave pressure (SW.meanP) 208 relative to a baseline pressure being atmospheric pressure. The mean pressure (SW.meanP) 208 may represent average of pressure samples divided by number of samples either during a rise time phase of the single pressure wave 209 or during an entire wave duration 210 of the single pressure wave 203. Amplitude (SW.dP) 211 of the single pressure wave 203 may represent differences in pressure between systolic maximum 206 and diastolic minimum pressures 205.

For determination of pressure stability levels 1008, the delta single pressure wave (dSW.x) related parameters 1007 are computed. With reference to FIGS. 7a-b, a change in mean pressure (dSW.meanP) 213, 214 between single pressure waves 203, for example between SWₙ₋₁ and SWₙ, represents change in absolute pressure between the single pressure waves 203. Moreover, a change in amplitude (dSW.dP) 215, 216 between the single pressure waves (n−1;n) represents change in internal signal relative pressure between the single pressure waves.

A pressure stability level 1008 refers to average value of any one of the single pressure wave parameters 1006 SW.meanP and SW.dP, and the single pressure waves 1005 included in the pressure stability level 1008 are based on any one of the respective delta single pressure wave parameters 1007 dSW.meanP and dSW.dP, the parameters being within selectable thresholds.

Pressure differences between pressure stability levels (SW.x.PSL.PD) 1009 refer to difference in average pressure of the pressure stability levels 1008.

Instability of baseline pressure 1013 refers to instability of reference pressure of the pressure sensor 1001, and reference pressure is an absolute pressure value. According to this disclosure, information about instability of baseline pressure is incorporated in the baseline pressure indicator plot 1010. The baseline pressure indicator (BPi) plot 1010 is created from the pressure stability level 1008, which refers to average value of either of the single pressure wave parameters SW.meanP and SW.dP 1006, and wherein the single pressure waves 1005 included in the pressure stability level 1008 are based on either of the respective delta single pressure wave parameters dSW.meanP and dSW.dP 1007, the parameters being within a first type of selectable thresholds. The baseline indicator plot 1010 is as well created from pressure differences between pressure stability levels 1009, which refer to difference in average pressure of the pressure stability levels 1008. Nearby pressure stability levels 1008 are merged into one pressure stability level if pressure difference between pressure stability levels 1009 is within selectable thresholds. Accordingly, information about instability of baseline pressure 1013 relies on information about pressure difference between different pressure stability levels 1009 of individual single wave parameter, such as SW.meanP 1006, incorporating a second type of thresholds, as indicated in 1011. Moreover, information about instability of baseline pressure 1013 relies on comparison of pressure stability levels 1009 of different types of single wave parameters 1006, such as comparing single wave parameters are SW.meanP and SW.dP, incorporating a third type of thresholds, as indicated in 1012.

The first type of selectable thresholds relate to pressure ranges of dSW.x 1008. The second type of selectable thresholds relate to pressure ranges of SW.x.PSL.PD of definable durations (SW.x.PSL.TD) 1011 calculated from the same type of single pressure wave (SW.x)-related parameters. A third type of selectable thresholds relates to ratios for combinations of pressure stability levels of different types of single pressure wave (SW.x)-related parameters 1012.

The first, second and third types of selectable set thresholds are created from previously established measurements and stored in a database.

The information about stability of baseline pressure may incorporate issuance of an alert 1014 in presence of at least one of: a) combinations of pressure differences 1009 between different of the pressure stability levels 1008 calculated from the same type of single pressure wave (SW.x)-related parameters, the pressure differences being outside a second type of selectable set thresholds 1011, reflecting deviations from nominal reference pressure differences, and b) relationships between different and simultaneous of the pressure stability levels (n−1; n) calculated from different types of single pressure wave (SW.x)-related parameters 1006 being outside a third type of selectable set thresholds 1012, reflecting deviations from nominal reference relationships. The alert 1014 being at least one of: a warning color of at least one part of the baseline indicator plot 1010 shown on an output monitor screen, warning noise by output means, and descriptive information provided by output means.

The second type of thresholds of a) 1011 and third type of thresholds of the b) 1012 are created from previously established measurements and stored in a database.

Aspects of the disclosure include a system for assessing stability of baseline pressure of a pressure sensor. This aspect is illustrated in FIG. 18. FIG. 18 illustrates a system for assessing stability of baseline pressure of a pressure sensor applied for sampling of continuous pressure signals, according to embodiments of the present disclosure.

More specifically, FIG. 18 illustrates a system 1101 for assessing stability of baseline pressure of a pressure sensor 1102 applied for sampling of continuous pressure signals 1103 originating from inside a human body or body cavity,

wherein the system 1101 comprises:
a pressure sensor 1102 configured to measure continuous pressure signals 1103 from the human body or body cavity at specific intervals,
transfer means 1104 configured to transfer the pressure signals 1103 from the pressure sensor 1102 to a sampling unit 1105,
a signal converter 1106 in communication with the sampling unit 1105 and configured to perform conversion of sampled pressure signals 1107, from the sampling unit 1105, into pressure-related digital data with a time reference 1108,
an identifier unit 1109 configured to receive the pressure-related digital data 1108 from the signal converter 1106 and identify therefrom single pressure waves 1110 related to cardiac beat-induced pressure waves,
a detector 1111 connected to an output of the identifier unit 1109 and configured to detect single pressure wave (SW.x)-related parameters 1112, being one or more of single wave mean pressure (SW.meanP) and single wave amplitude (SW.dP), and
a computing device 1113 connected to an output of the detector 1111 and configured to compute one or more of delta single pressure wave (dSW.x)-related parameters 1114 representing differences in single pressure wave (dSW.x)-related parameters 1114 being one or more of change in mean pressure (dSW.meanP) and change in amplitude (dSW.dP) between a consecutive number of single pressure waves (n−1;n) 1110,
wherein a calculation unit 1115 is connected to an output of the computing device 1113 and configured to calculate pressure stability levels (SW.x.PSL) 1116, each pressure stability level being created from consecutive single pressure waves 1110 having any one of delta single pressure wave (dSW.x)-related parameters dSW.meanP and dSW.dP 1114 within a first set of thresholds, the first set of

thresholds referring to defined pressure ranges of any one of the parameters dSW.meanP and dSW.dP 1114, and wherein each pressure stability level refers to an average of any one of the single pressure wave (SW.x)-related parameters SW.meanP and SW.dP 1112,
wherein a determination unit 1118 is connected to an output of the calculation unit 1115 and configured to determine pressure differences (SW.x.PSL.PD) 1119 between different of the pressure stability levels (n−1;n) (SW.x.PSL) 1116,
wherein the pressure stability levels (SW.x.PSL) 1116 have definable time durations (SW.x.PSL.TD) relating to a time duration of the pressure stability levels (SW.x.PSL) 1116,
wherein a presentation unit 1120 is connected to an output of the determination unit 1118 and configured to present baseline pressure indicator (BPi) plots 1121, being created from the pressure stability levels (SW.x.PSL) 1116 and with beginning pressure differences and ending pressure differences (SW.x.PSL.PD) 1119 for each pressure stability level (SW.x.PSL) 1116, the beginning pressure difference being defined as the difference between a present pressure stability level and a previous pressure stability level and the ending pressure difference being defined as a difference between a present pressure stability level and a next pressure stability level,
wherein the BPi plots provide information about stability of baseline pressure of the pressure sensor and are a function of at least one of:

a) combinations of the pressure differences between different of the pressure stability levels (SW.x.PSL) 1116 calculated from a same type of single pressure wave (SW.x)-related parameters, the pressure differences being outside or inside a second set of thresholds 1122, reflecting deviations from nominal reference pressure differences, and

b) relationships between different and simultaneous pressure stability levels (n−1; n) calculated from different types of single pressure wave (SW.x)-related parameters 1112, the relationships being outside or inside a third set of thresholds 1123, reflecting deviations from nominal reference relationships, and
wherein the presentation unit 1120 is configured to indicate if parameters of a) and/or b) are outside the second set and/or the third set of thresholds and thereby define instability of baseline pressure of the pressure sensor 1124.

The pressure sensor 1102 is configured to measure intracranial pressure (ICP) or arterial blood pressure (ABP) signals.

The body cavity may be a cranio-spinal cavity providing for measurement of intracranial pressure (ICP) or a blood-vessel compartment providing for measurement of arterial blood pressure (ABP). The pressure sensor(s) 1102 used may be configured for measuring of intracranial pressure (ICP) from the cranio-spinal cavity or arterial blood pressure (ABP) signals from the blood-vessel compartment. The pressure sensor 1102 may be implantable for a temporary period or an extended period of weeks or months. When using a pressure sensor 1102 implantable for an extended period of weeks or months, transfer means 1104 for continuous pressure signals may be of wireless type.

Regarding the identified single pressure waves 1110 and single pressure wave (SW.x)-related parameters 1112 further details are given in FIGS. 7a-b. Hence, mean pressure 204 detected by the detector represents a static pressure being mean single wave pressure (SW.meanP) 208 relative to a baseline pressure being atmospheric pressure. The mean pressure 208 may represent average of pressure samples divided by number of samples either during a rise time phase of the single pressure wave 205 or during an entire wave duration 110 of the single pressure wave 203. Further, the amplitude (SW.dP) 211 detected by the detector 1111 represents differences in pressure between systolic maximum 206 and diastolic minimum pressures 205.

Change in mean pressure (dSW.meanP) 213, 214 between selectable numbers of single pressure waves 203 computed by the computing device 1113 represents change in absolute pressure between the single pressure waves. The change in amplitude (dSW.dP) 215, 216 between selectable numbers of single pressure waves 203 represents change in internal signal relative pressure between the single pressure waves.

According to this system, a database 1125 of associations between the delta single pressure wave (dSW.x)-related parameters change in mean pressure (dSW.meanP) and change in amplitude (dSW.dP) may be created from previously established measurements, which may provide for empirical basis for defining thresholds for alerts.

The first type of selectable thresholds 1116 relate to pressure ranges of dSW.x. The second type of selectable thresholds 1122 relate to pressure ranges of SW.x.PSL.PD of definable durations (SW.x.PSL.TD) of the same type of single pressure wave (SW.x)-related parameters 1112. The third type of selectable thresholds 1123 relate to ratios for combinations of pressure stability levels (SW.x.PSL) 1116 of different types of single pressure wave (SW.x)-related parameters 1112.

Instability of baseline pressure 1124 refers to instability of reference pressure of the pressure sensor, and reference pressure is an absolute pressure value. In this context, information about instability of baseline pressure 1124 is incorporated in the baseline pressure indicator plot 1121. The baseline pressure indicator plot is created from pressure stability levels 1116, which refer to average value of either of the single pressure wave parameters SW.meanP and SW.dP 1112, and wherein the single pressure waves 1110 included in the pressure stability level 1116 are based on either of the respective delta single pressure wave parameters dSW.meanP and dSW.dP, the parameters being within selectable thresholds. The baseline pressure indicator plot 1121 is also created from the pressure differences between pressure stability levels 1119 refer to difference in average pressure of the pressure stability levels. Nearby pressure stability levels 1116 may be merged into one pressure stability level if pressure difference 1119 between pressure stability levels 1116 is within selectable ranges of the second type of selectable thresholds 1122. Therefore, information about stability of baseline pressure 1121 incorporates information of pressure difference 1119 between different pressure stability levels 1116 of a selectable number of the single wave parameters, such as the single wave parameters are SW.meanP and SW.dP.

The information about stability of baseline pressure may incorporate issuance of an alert 1126 by the presentation unit 1120 in presence of at least one of: a) the pressure difference between different stability levels 1119 is outside the second type of thresholds 1122, reflecting deviations from nominal reference pressure differences, or if b) relationship between pressure stability levels 1116 of different single pressure wave (SW.x)-related parameters 1112 being outside the third type of thresholds 1123, reflecting deviations from nominal reference relationships. The alert 1126 may be at least one of: a warning color of at least one part of the baseline pressure indicator plot 1121 shown on an output monitor screen of the presentation unit 1120, a warning noise from the presentation unit 1120, and a descriptive information displayed or printed by the presentation unit 1120.

Moreover, the second type of thresholds of the a) 1122, and third type of thresholds of the b) 1123 are created from previously established measurements and stored in a database 1125.

Aspects of the disclosure relate to usage of pressure sensors for ICP, ABP and CPP measurements for detection of baseline pressure instability. These pressure sensors are currently used for measurements of static and pulsatile pressures, while not for measurements of baseline pressure instability.

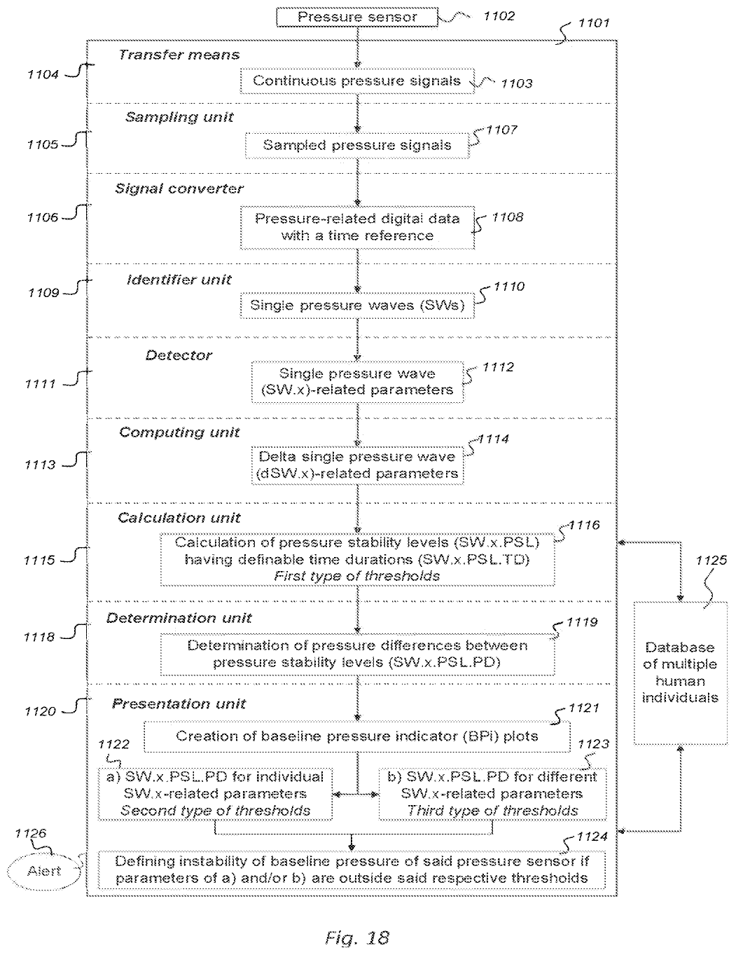

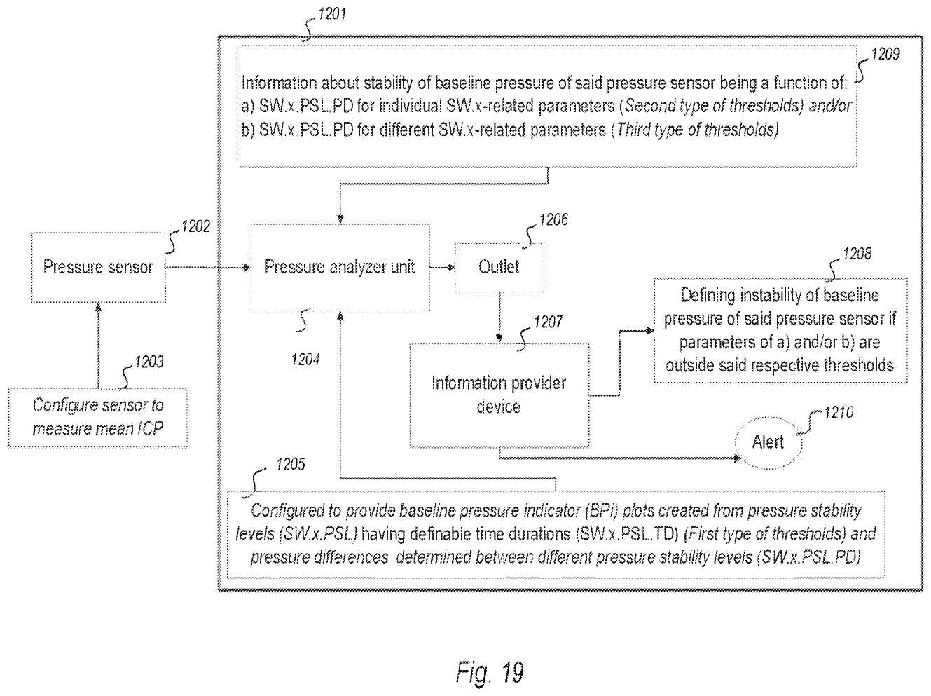

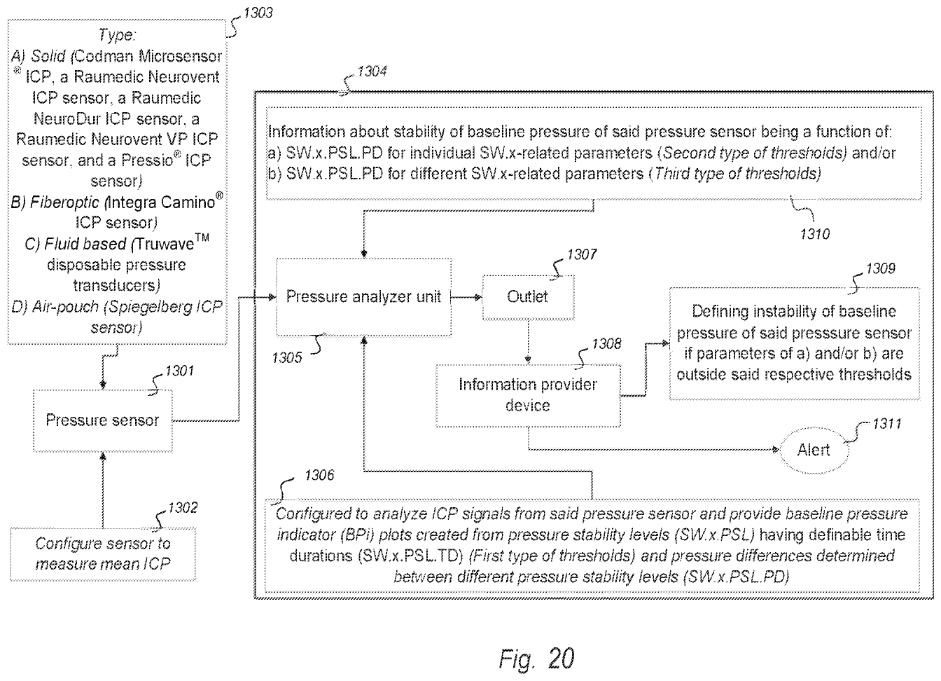

Aspects of the disclosure relate to a pressure analyzing system 1201, 1304 (shown in FIGS. 19 and 20, respectively) to assess intracranial pressure (ICP) in a human. FIG. 19 illustrates a pressure analyzing system configured to assess according to embodiments of the present disclosure. FIG. 20 illustrates an apparatus comprising a pressure sensor and a pressure analyzer unit communicating with the pressure sensor, configured to assess ICP, according to embodiments of the present disclosure The system 1201, 1304 comprises:
a pressure sensor 1202, 1301 that is insertable into a cranio-spinal cavity or in communication with fluid of the cranio-spinal cavity, the pressure sensor 1202, 1301 being configured to measure ICP signals 1203, 1302, which represent differences in pressure between atmospheric pressure and pressure inside the cranio-spinal cavity, and
a pressure analyzer unit 1204, 1305 in communication with the pressure sensor 1202, 1301, the pressure analyzer unit 1204, 1305 being configured to:

process and analyze the ICP signals from the pressure sensor 1202, 1301,

based on the processing and analyzing of the ICP signals, provide one or more baseline pressure indicator (BPi) plots 1205, 1306 created from pressure stability levels (SW.x.PSL) of predefined time durations (SW.x.PSL.TD), calculated from single pressure wave (SW.x)-related parameters from a definable number of single pressure waves having delta single pressure wave (dSW.x)-related parameters within a first set of thresholds 1205, 1306, the first set of thresholds referring to defined pressure ranges of any one of the parameters dSW.meanP and dSW.dP 1205, 1306 and beginning pressure differences and ending pressure differences for each pressure stability level (SW.x.PSL.PD) 1205, 1306, the beginning pressure difference being defined as a difference between a present pressure stability level and a previous pressure stability level and the ending pressure difference being defined as a difference between a present pressure stability level and a next pressure stability level,
wherein the pressure analyzer unit 1204, 1305 has an outlet 1206, 1307 and information provider device 1207, 1308 configured to provide information 1209, 1310 about the stability of baseline pressure of the pressure sensor from the baseline pressure indicator plot 1205, 1306, the information being a function of at least one of:

a) combinations of pressure differences between different of the pressure stability levels calculated from the same type of single pressure wave (SW.x)-related parameters, the pressure differences being outside or inside a second set of thresholds, reflecting deviations from nominal reference pressure differences, and

b) relationships between different and simultaneous pressure stability levels (n−1; n) calculated from different types of single pressure wave (SW.x)-related parameters, the relationships being outside or inside a third set of thresholds, reflecting deviations from nominal reference relationships, wherein parameters of a) and/or b) outside the second and/or the third sets of thresholds define instability of baseline pressure of the pressure sensor, and
wherein the information provider device 1207, 1308 is configured to indicate if parameters of a) and/or b) 1209, 1310 are outside the second and/or the third sets of thresholds based on output from the pressure analyzer unit 1204, 1305, and thereby define a presence of instability of baseline pressure of the pressure sensor 1208, 1309.

The pressure sensor 1202, 1301 is of a sensor type that is configured to measure ICP signals 1203, 1302 within one of: a cerebrospinal fluid compartment and a brain tissue compartment, inside or outside the dura of the cranio-spinal cavity. The pressure sensor 1202, 1301 may be a) a solid pressure sensor, b) a fiberoptic pressure sensor, c) a fluid-based pressure sensor, and d) an air-pouch sensor.

Moreover, the pressure analyzing system 1201, 1304 includes a pressure analyzer unit 1204, 1305 that is configured to enable 1205, 1306:
from the ICP signals, identification of single pressure waves related to cardiac beat-induced pressure waves,
detection of at least two single pressure wave (SW.x)-related parameters selectable from one or more of mean pressure (SW.meanP) and amplitude (SW.dP), based on detection, computation of delta single pressure wave (dSW.x)-related parameters between a definable number of single pressure waves (n−1;n), representing differences in single pressure wave (dSW.x)-related parameters selectable from one or more of change in mean pressure (dSW.meanP) and change in amplitude (dSW.dP) between a selectable number of single pressure waves (n−1;n),
calculation of pressure stability levels (SW.x.PSL) of the single pressure wave (SW.x)-related parameter from a selectable number of single pressure waves having delta single pressure wave (dSW.x)-related parameters within the first type of selectable set thresholds 1205, 1306 and

a) determination of pressure differences between different of the pressure stability levels (SW.x.PSL.PD) 1205, 1306, calculated from the same type of single pressure wave (SW.x)-related parameters, the pressure differences being outside or inside a second set of thresholds, and

b) determination of relationships between different and simultaneous pressure stability levels (n−1; n) calculated from different types of single pressure wave (SW.x)-related parameters, the relationships being outside or inside a third set of thresholds,

and the pressure stability levels and pressure differences together creating a baseline pressure indicator plot 1205, 1306, which incorporates information from the information provider device 1207, 1308 about stability of baseline pressure 1208, 1309.

The first set of thresholds 1205, 1306 relate to pressure ranges of dSW.x. The second set of thresholds 1209, 1310 relate to pressure ranges of SW.x.PSL.PD of definable durations (SW.x.PSL.TD) of the same type of single pressure wave (SW.x)-related parameters. The third set of thresholds 1209, 1310 relate to ratios for combinations of pressure stability levels of different types of single pressure wave (SW.x)-related parameters.

The system 1201, 1304 wherein information about stability of baseline pressure 1208, 1309 is provided by the pressure analyzer outlet 1206, 1307 incorporates issuance of an alert 1210, 1311 from the information provider device 1207, 1308 in presence of at least one of:

a) the pressure difference between different pressure stability levels 1205, 1306 being outside second set of thresholds, reflecting deviations from nominal reference pressure differences, and

b) relationship between the pressure stability levels 1205, 1306 of different types of single pressure wave (SW.x)-related parameters being outside third set of thresholds, reflecting deviations from nominal reference relationships.

The alert 1210, 1311 is at least one of: a warning color of at least one part of the baseline indicator plot shown on an output monitor screen of the information provider 1207, 1308, warning noise by output means of the information provider 1207, 1308, and descriptive information provided by output means of the information provider 1207, 1308.

The information output 1208, 1309 from the pressure analyzer unit 1204, 1305 provides a basis for any subsequent correction of mean ICP measurements.

Some additional details about the pressure sensor 1202, 1301 of the system 1201, 1304 are illustrated in FIG. 20. The pressure sensor 1301 is of a type being configured to measure ICP signals 1302 within one of: a cerebrospinal fluid compartment and a brain tissue compartment, inside or outside the dura of the cranio-spinal cavity. Further, the pressure sensor 1301 is selected from the following types 1303: a) a solid pressure sensor, or b) a fiberoptic pressure sensor, or c) a fluid-based pressure sensor. For example, the pressure sensor is a selected one of: a) a solid pressure sensor selected from one of: a Codman Microsensor ° ICP, a Raumedic Neurovent ICP sensor, a Raumedic NeuroDur ICP sensor, a Raumedic Neurovent VP ICP sensor, and a Pressio® ICP sensor, or b) a fiberoptic pressure sensor selected from one of Integra Camino® ICP sensor, or c) a fluid-based pressure sensor selected one of Truwave™ disposable pressure transducers, or d) an air-pouch sensor selected from one of Spiegelberg intraparenchymal probe.

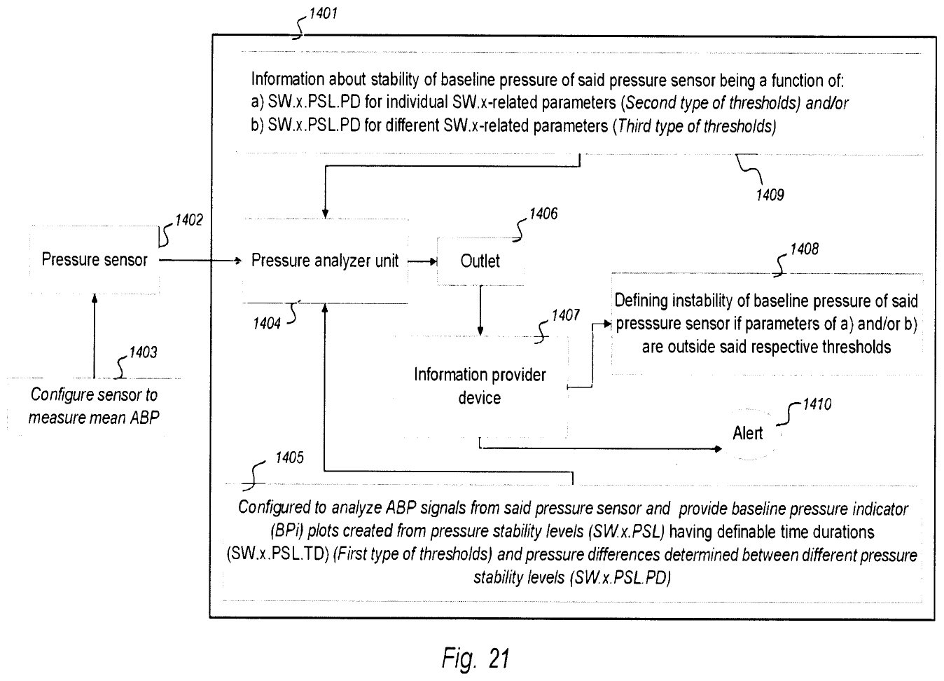

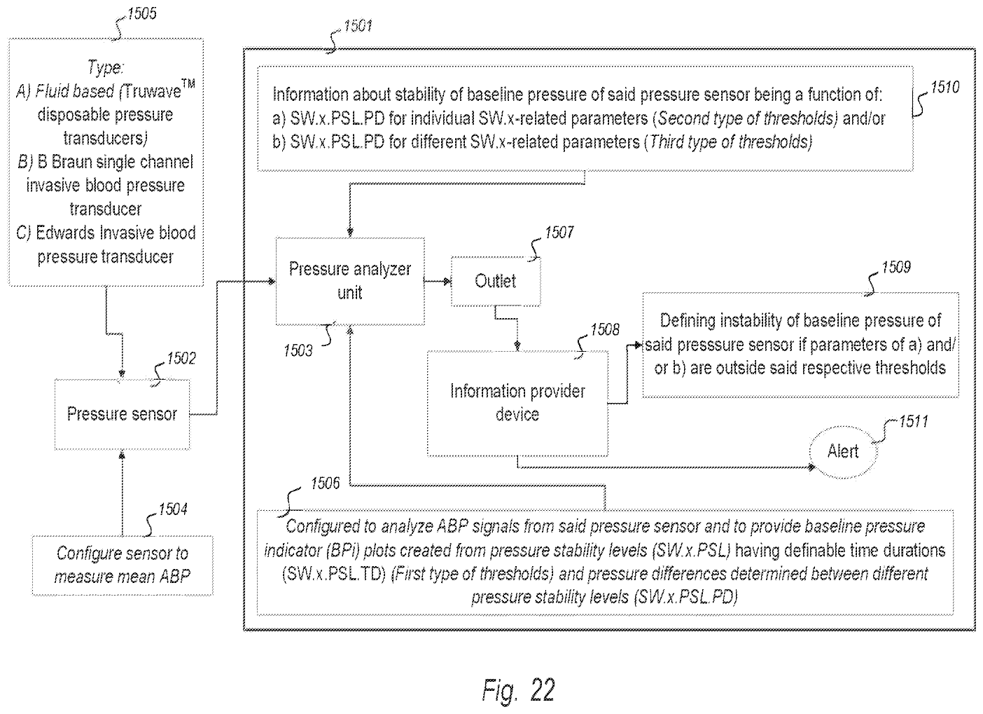

Aspects of the disclosure relate to a pressure analyzing system 1401, 1501 (shown in FIGS. 21 and 22, respectively) to assess arterial blood pressure (ABP) in a human. FIG. 21 illustrates a pressure analyzing system configured to assess ABP, according to embodiments of the present disclosure. FIG. 22 illustrates an apparatus comprising a pressure sensor in communication with a pressure analyzer unit, configured to assess ABP, according to embodiments of the present disclosure.

The system 1401, 1501 comprises:
a pressure sensor 1402, 1502 that is insertable into a blood-vessel compartment or in communication with fluid of the blood-vessel compartment, the pressure sensor being configured to measure ABP signals 1403, 1504, which represents differences in pressure between atmospheric pressure and pressure inside the blood-vessel compartment, and
a pressure analyzer unit 1404, 1503 in communication with the pressure sensor 1402, 1502, the pressure analyzer unit 1404, 1503 being configured to: process and analyze the ABP signals from the pressure sensor 1402, 1502, and

based on the processing and analyzing of the ABP signals, provide one or more baseline pressure indicator plots 1405, 1506 created from pressure stability levels (SW.x.PSL) of predefined time durations (SW.x.PSL.TD), calculated from single pressure wave (SW.x)-related parameters from a definable number of single pressure waves having delta single pressure wave (dSW.x)-related parameters within a first set of thresholds 1405, 1506, the first set of thresholds referring to defined pressure ranges of any one of the parameters dSW.meanP and dSW.dP, and beginning pressure differences and ending pressure differences for each pressure stability level (SW.x.PSL.PD) 1405, 1506, the beginning pressure difference being defined as a difference between a present pressure stability level and a previous pressure stability level and the ending pressure difference being defined as a difference between a present pressure stability level and a next pressure stability level,

wherein the pressure analyzer unit 1404, 1503 has an outlet 1406, 1507 and an information provider device 1407, 1508 configured to provide information 1409, 1510 about the stability of baseline pressure of the pressure sensor 1408, 1509 from the baseline pressure indicator (BPi) plot 1405, 1506, the information being a function of at least one of:

a) combinations of pressure differences between different of the pressure stability levels calculated from a same type of single pressure wave (SW.x)-related parameters, the pressure differences being outside or inside a second set of thresholds, reflecting deviations from nominal reference pressure differences, and

b) relationships between different and simultaneous pressure stability levels (n−1; n) calculated from different types of single pressure wave (SW.x)-related parameters, the relationships being outside or inside a third set of thresholds, reflecting deviations from nominal reference relationships, wherein parameters of a) and/or b) outside the second set and/or the third set of thresholds define instability of baseline pressure of the pressure sensor, and

wherein the information provider device 1407, 1508, based on output from the pressure analyzer unit 1404, 1503, is configured to indicate if parameters of a) and/or b) 1409, 1510 are outside the second set and/or the third set of thresholds and thereby define a presence of instability of baseline pressure of the pressure sensor 1408, 1509.

The pressure sensor 1402, 1502 of this system 1401, 1501 may be of a type being configured to measure ABP signals 1403, 1504 inside an arterial blood vessel or in a body-external blood flow conduit communicating with the arterial blood vessel, e.g., an artery. Hence, the pressure sensor 1402, 1502 may be a fluid-based pressure sensor.

A first set of thresholds 1406, 1506 relates to pressure ranges of dSW.x. A second set of thresholds 1409; 1510 relates to pressure ranges of SW.x.PSL.PD of definable durations (SW.x.PSL.TD) of the same type of single pressure wave (SW.x)-related parameters. A third set of thresholds 1409; 1510 relates to ratios for combinations of pressure stability levels of different types of single pressure wave (SW.x)-related parameters.

The system 1401, 1501 incorporates a pressure analyzer unit 1404, 1503 is configured to enable:
from the ABP signals, identification of pressure waves related to cardiac beat-induced pressure waves,
detection of at least two single pressure wave (SW.x)-related parameters selectable from one or more of mean pressure (SW.meanP) and amplitude (SW.dP),
based on the detection, computation of delta single pressure wave (dSW.x)-related parameters between a selectable number of single pressure waves (n−1;n), representing differences in single pressure wave (dSW.x)-related parameters selectable from one or more of change in mean pressure (dSW.meanP) and change in amplitude (dSW.dP) between a definable number of single pressure waves (n−1;n),
calculation of pressure stability levels (SW.x.PSL) of the single pressure wave (SW.x)-related parameter from a definable number of single pressure waves having delta single pressure wave (dSW.x)-related parameters within selectable thresholds, and
determination of pressure differences between different of the pressure stability levels (SW.x.PSL.PD), each of the pressure stability levels incorporating a definable number of single pressure waves, and the pressure stability levels and pressure differences together creating a baseline pressure indicator plot, which incorporates information about stability of baseline pressure.

The information about stability of baseline pressure 1408,1509 provided by the pressure analyzer outlet 1406, 1507 incorporates issuance of an alert 1410, 1511 from the information provider device 1407, 1508 in presence of at least one of 1409, 1510:

a) the pressure difference between different pressure stability levels is outside the second set of thresholds, reflecting deviations from nominal reference pressure differences, and if

b) relationship between the pressure stability levels of different types of single pressure wave (SW.x)-related parameters is outside the third set of thresholds, reflecting deviations from nominal reference relationships.

An alert 1410, 1511 may be at least one of: a warning color of at least one part of the baseline indicator plot shown on an output monitor screen of the information provider 1407, 1508, warning noise by output means of the information provider 1407, 1508, and descriptive information provided by output means of the information provider 1407, 1508.

The information delivered from the analyzer unit 1404, 1503 of the system 1401, 1501 provides a basis for subsequent correction of mean ABP measurements 1403, 1504.

Some additional embodiments of the system described in FIG. 21, are further illustrated in FIG. 22. In one embodiment of the system 1401, 1501, the pressure sensor 1402, 1502 is a fluid-based sensor selected one of: a) Truwave™ disposable pressure transducers, or b) B Braun single channel invasive blood pressure transducer, or c) Edwards Invasive blood pressure transducer 1505.

The pressure sensor 1502 may be a fluid-based pressure sensor selected from one of: Truwave pressure transducers, B Braun single channel invasive blood pressure transducer, and Edwards Invasive blood pressure transducer 1505.

The pressure sensor 1502 can be configured for implantation for a shortened or temporary time period or for an extended period of weeks or months. Further, the pressure signals obtainable by the pressure sensor 1502 are wireless transferred when the sensor is implanted for the extended period.

Determination of cerebral perfusion pressure (CPP) is extensively used for surveillance of patients with various kinds of brain disease. This parameter is determined according to this formula: CPP=Mean ABP—Mean ICP. The baseline pressure instability may affect both mean ABP and mean ICP, and impact the interpretation of CPP.

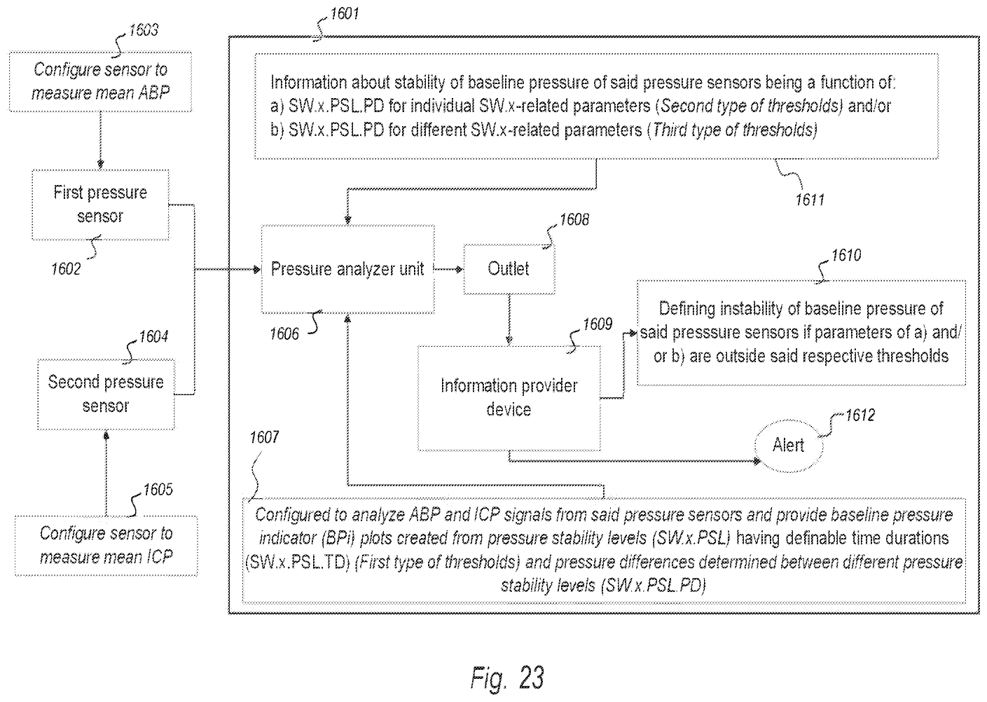

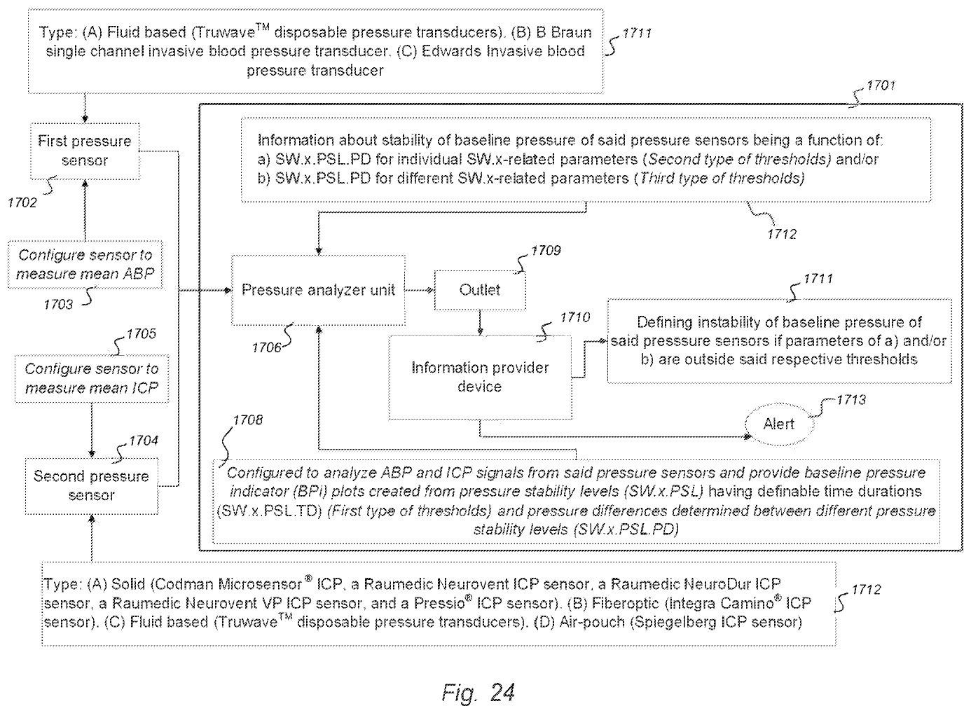

Aspects of the disclosure relate to a pressure analyzing system 1601, 1701 (shown in FIGS. 23 and 24, respectively) to assess cerebral perfusion pressure (CPP) in a human, i.e. mean arterial blood pressure (ABP) minus mean intracranial pressure (ICP). FIG. 23 illustrates a pressure analyzing system configured to assess CPP, according to embodiments of the present disclosure. FIG. 24 illustrates an apparatus in a pressure analyzing system to assess cerebral perfusion pressure (CPP) in a human, according to embodiments of the present disclosure.

The systems 1601, 1701 comprise:
a first pressure sensor 1602, 1702 that is insertable into a blood-vessel compartment or in communication with fluid of the blood-vessel compartment, the pressure sensor being configured to measure mean ABP signals 1603, 1703, which represent differences in pressure between atmospheric pressure and pressure inside the blood-vessel compartment,
a second pressure sensor 1604, 1704 that is insertable into a cranio-spinal cavity or in communication with fluid of the cranio-spinal cavity, the pressure sensor being configured to measure mean ICP signals 1605, 1705, which represent differences in pressure between atmospheric pressure and pressure inside the cranio-spinal cavity, and
a pressure analyzer unit 1606, 1706 in communication with the first and second pressure sensors configured to measure mean pressure 1603, 1703, 1605, 1705, the analyzer unit 1606, 1706 being configured to process and analyze pressure measurements of arterial blood pressure (ABP) and intracranial pressure (ICP) signals from the pressure sensors 1602, 1702, 1604, 1704,

wherein the pressure analyzer unit 1606, 1706 is further configured to provide baseline pressure indicator (BPi) plots 1607, 1708 from the ABP and ICP signals, the BPi plots being created from pressure stability levels (SW.x.PSL) of definable time durations (SW.x.PSL.TD), calculated from single pressure wave (SW.x)-related parameters from a predefined number of single pressure waves having delta single pressure wave (dSW.x)-related parameters within a first set of thresholds 1607, 1708 the first set of thresholds referring to defined pressure ranges of any one of the parameters dSW.meanP and dSW.dP, and with beginning pressure differences and ending pressure differences for each pressure stability levels (SW.x.PSL.PD) 1607, 1708, the beginning pressure difference being defined as a difference between a present pressure stability level and a previous pressure stability level and the ending pressure difference being defined as a difference between a present pressure stability level and a next pressure stability level,

wherein the pressure analyzer unit 1606, 1706 has an outlet 1608, 1709 and information provider device 1609, 1711 configured to provide information 1611, 1712 about the stability of baseline pressure of the pressure sensor from the baseline pressure indicator plots 1607, 1708, the information being a function of at least one of:

a) combinations of pressure differences between different of the pressure stability levels (SW.x.PSL) calculated from a same type of single pressure wave (SW.x)-related parameters, the pressure differences being outside or inside a second set of thresholds, reflecting deviations from nominal reference pressure differences, and

b) relationships between different and simultaneous pressure stability levels (n−1; n) calculated from different types of single pressure wave (SW.x)-related parameters, the relationships being outside or inside a third set of thresholds, reflecting deviations from nominal reference relationships, and

wherein parameters of a) and/or b) outside the second set and/or the third set of thresholds define instability of baseline pressure of the pressure sensor, and

wherein the information provider device 1609, 1710 based on output from the pressure analyzer unit 1606, 1706 is configured to indicate if parameters of a) and/or b) 1611, 1712 are outside the respective thresholds and thereby define presence of instability of baseline pressure of the pressure sensors 1610, 1711.

The first pressure sensor 1602, 1702 is of a type being configured to measure ABP 1603, 1703 inside an arterial blood vessel or in a body-external blood flow conduit communicating with an arterial blood vessel, and the second pressure sensor 1604, 1704 is of a type being configured to measuring ICP 1605, 1705 within one of: a cerebrospinal fluid compartment, a brain tissue compartment, inside or outside the dura of the cranio-spinal cavity.

The first pressure sensor 1602, 1702 of the system configured to measure ABP 1603, 1703 may be a fluid based sensor, and wherein the second pressure sensor configured to measure ICP is one of: a) a solid pressure sensor, b) a fiberoptic pressure sensor, c) a fluid-based pressure sensor, and d) an air-pouch pressure sensor.

A first set of thresholds 1607, 1708 relates to pressure ranges of dSW.x. A second set of thresholds 1611, 1712 relates to pressure ranges of SW.x.PSL.PD of definable durations (SW.x.PSL.TD) of the same type of single pressure wave (SW.x)-related parameters. A third set of thresholds 1611, 1712 relates to ratios for combinations of pressure stability levels of different types of single pressure wave (SW.x)-related parameters.

The pressure analyzer unit 1606, 1706 of the system 1601, 1701 is configured to enable 1607, 1708:
identifying from the ABP and ICP signals ABP and ICP single pressure waves related to cardiac beat-induced pressure waves,
from each of the ABP and ICP signals, detecting at least two single pressure wave (SW.x)-related parameters selectable from one or more of mean pressure (SW.meanP) and amplitude (SW.dP),
based on the detection, computation of delta single pressure wave (dSW.x)-related parameters between a selectable number of single pressure waves (n−1;n), representing differences in single pressure wave (dSW.x)-related parameters selectable from one or more of change in mean pressure (dSW.meanP) and change in amplitude (dSW.dP) between a selectable number of single pressure waves (n−1;n),
calculation of pressure stability levels (SW.x.PSL) of the single pressure wave (SW.x)-related parameter from a definable number of single pressure waves having delta single pressure wave (dSW.x)-related parameters within a first type of selectable thresholds, and

a) determination of pressure differences between different of the pressure stability levels (SW.x.PSL.PD), calculated from the same type of single pressure wave (SW.x)-related parameters, the pressure differences being outside or inside a second type of thresholds,

b) determination of relationships between different and simultaneous pressure stability levels (n−1; n) calculated from different types of single pressure wave (SW.x)-related parameters, the relationships being outside or inside a third type of selectable set thresholds,

and the pressure stability levels and pressure differences together creating a baseline pressure indicator plot, which incorporates information about stability of baseline pressure.

The information about stability of baseline pressure 1610, 1711 provided by the pressure analyzer outlet 1608, 1709 incorporates issuance of an alert 1612, 1713 from the information provider device 1609, 1710 in presence of at least one of 1611, 1712:

a) the pressure difference between different pressure stability levels being outside the second set of thresholds, reflecting deviations from nominal reference pressure differences and,

b) relationship between the pressure stability levels of different single pressure wave (SW.x)-related parameters is outside the third set of thresholds, reflecting deviations from nominal reference relationships.

An alert 1612, 1713 may be at least one of: a warning color of at least one part of the baseline indicator plot shown on an output monitor screen of the information provider 1609, 1710, warning noise by output means of the information provider 1609, 1710, and descriptive information provided by output means of the information provider 1609, 1710.

The information 1610, 1711 from the analyzer unit outlet 1608, 1709 provides a basis for any subsequent correction of mean CPP, mean ABP, and mean ICP measurements.

The first pressure sensor 1702 of the apparatus 1701 is configured to measure

ABP 1703 and is a fluid based sensor selected from one of 1711: Truwave disposable pressure transducers, or B Braun single channel invasive blood pressure transducer, or Edwards Invasive blood pressure transducer. The second pressure sensor 1704 is configured to measuring ICP 1705 and is 1712: a) a solid pressure sensor selected from one of: a Codman Microsensor ° ICP, a Raumedic Neurovent ICP sensor, a Raumedic NeuroDur ICP sensor, a Raumedic Neurovent VP ICP sensor, and a Pressio® ICP sensor, or b) a fiberoptic pressure sensor selected from one of Integra Camino® ICP sensors, or c) a fluid-based pressure sensor selected from one of Truwave™ disposable pressure transducers, B Braun single channel invasive blood pressure transducer, and Edwards Invasive blood pressure transducer, or d) an air-pouch sensor selected from one of Spiegelberg intraparenchymal probe.

Aspects of the disclosure relate to means and methods for correcting mean pressure alterations caused by instability of baseline pressure of a pressure sensor. This is relevant for pressure sensors used for measuring pressure inside a body cavity. The present disclosure addresses primarily invasive intracranial pressure (ICP) and arterial blood pressure (ABP) though this represents no limitation of the scope of the disclosure.

In some embodiments, a method for correcting single wave mean pressure (SW.meanP) is disclosed. FIG. 25 illustrates some main elements. FIG. 25 illustrates a method for correcting mean pressure alterations caused by instability of baseline pressure of a pressure sensor 1801 applied for sampling of continuous pressure signals 1802 originating from inside a human body or body cavity, samples of the pressure signals from the sensor being obtainable at specific intervals, and being convertible into pressure-related digital data with a time reference 1803,

the method comprising:

from the digital data identification of single pressure waves 1804 related to cardiac beat-induced pressure waves,

detection of single pressure wave (SW.x)-related parameters 1805, selectable from one or more of mean pressure (SW.meanP) and amplitude (SW.dP), and

based on the detection, computation of one or more delta single pressure wave (dSW.x)-related parameters 1806 between a selectable number of single pressure waves (n−1;n), representing differences in single pressure wave (dSW.x)-related parameters, selectable from one or more of change in mean pressure (dSW.meanP) and change in amplitude (dSW.dP) between a consecutive number of single pressure waves (n−1;n),

wherein pressure stability levels (SW.x.PSL) are created 1807, each pressure stability level being created from consecutive single pressure waves (SW.x) having any one of delta single pressure wave (dSW.x)-related parameters dSW.meanP and dSW.dP 1806 within a first set of selectable thresholds 1807, the first set of thresholds referring to defined pressure ranges of any one of the parameters dSW.meanP and dSW.dP, and

wherein a pressure stability level 1807 refers to average of any one of the single pressure wave (SW.x)-related parameters SW.meanP and SW.dP,

wherein pressure differences between different pressure stability levels (n−1; n) (SW.x.PSL.PD) are determined 1808, each of the pressure stability levels 1807 having definable time durations (SW.x.PSL.TD) relating to the time duration of the pressure stability levels (SW.x.PSL),incorporating a definable number of single pressure waves,

wherein the pressure stability levels (SW.x.PSL) of definable time durations (SW.x.PSL.TD) and with beginning and ending pressure differences (SW.x.PSL.PD) for each pressure stability level (SW.x.PSL), together creating a baseline pressure indicator (BPi) plot 1809, the beginning pressure difference being defined as the difference between a present and a previous pressure stability level and the ending pressure difference being defined as the difference between a present and a next pressure stability level,
wherein information about stability of baseline pressure of the pressure sensor being a function of at least one of:

a) combinations of pressure differences between different of the pressure stability levels calculated from the same type of single pressure wave (SW.x)-related parameters, the pressure differences being outside or inside a second set of thresholds 1810, reflecting deviations from nominal reference pressure differences, and

b) relationships between different and simultaneous pressure stability levels (n−1; n) calculated from different types of single pressure wave (SW.x)-related parameters, the relationships being outside or inside a third set of thresholds 1811, reflecting deviations from nominal reference relationships, and

wherein parameters of a) and/or b) outside the second set and/or the third set of thresholds define instability of baseline pressure of the pressure sensor 1812, and

wherein levels of the mean pressure 1805 related to baseline pressure instability are corrected 1813 as a function of the pressure difference between pressure stability levels (SW.x.PSL.PD) 1808, the corrections 1813 being selectable according to defined criteria, and wherein the corrected mean pressures are presented 1814.

The body cavity may be a cranio-spinal cavity providing for measurement of intracranial pressure (ICP) or a blood-vessel compartment providing for measurement of arterial blood pressure (ABP). The pressure sensor(s) 1801 used may be configured to measure ICP or ABP signals, and may be implanted for a shortened or temporary time period or for an extended period of weeks or months. When using a sensor 1801 implantable for the extended period, the pressure signals obtainable by the implanted pressure sensor 1801 are transferred wireless.

Regarding single pressure wave parameters 1805, mean pressure represents a static pressure being mean single wave pressure (SW.meanP) relative to a baseline pressure being atmospheric pressure. The mean pressure may represent average of pressure samples divided by number of samples either during a rise time phase 209 of the single pressure wave 203 or during an entire wave duration 210 of the single pressure wave 203. The amplitude (SW.dP) 211 may represent differences in pressure between systolic maximum 206 and diastolic minimum pressures 205. For further details, it is referred to FIGS. 7a-c.

A change in mean pressure (dSW.meanP) 1806 is between a definable number of single pressure waves (n−1;n), and represents change in absolute pressure between the single pressure waves. Further, the change in amplitude (dSW.dP) 1806 is between a definable number of single pressure waves (n−1;n), and represents change in internal signal relative pressure between the single pressure waves.

The pressure stability level 1807 refer to average value of either of the single pressure wave parameters SW.meanP and SW.dP, and wherein the single pressure waves included in the pressure stability level are based on either of the respective delta single pressure wave parameters dSW.meanP and dSW.dP 1806, the parameters being within the first type of selectable thresholds. The pressure differences between pressure stability levels 1808 refer to difference in average pressure of the pressure stability levels. Nearby pressure stability levels 1807 are merged into one pressure stability level if pressure difference 1808 between pressure stability levels is within selectable thresholds.

The information about stability of baseline pressure incorporates information of pressure difference 1808 between different pressure stability levels of a definable number of the single pressure wave parameter SW.meanP, and/or information about stability of baseline pressure 1807 as comparison of pressure stability levels of different single wave parameter, the single wave parameters are SW.meanP and SW.dP.

Different methods may be used for correcting mean pressure alterations caused by baseline pressure instability. Hence, mean pressure (SW.MeanP) is corrected by subtracting the mean pressure (SW.MeanP) level of a selectable number of single pressure waves of a pressure stability level by the first pressure difference of the pressure stability level. The corrected mean pressure (SW.MeanP) is provided as descriptive information such as corrected mean pressure (SW.MeanP_{Corr}) in addition to the measured mean pressure (SW.MeanP).

Empirical data were established from database information 1815. Based on empirical observations corrections of mean pressure become possible.

The method may incorporate an output step for presentation of corrected mean pressure 1814. The correction of mean pressure may be provided as descriptive information such as corrected mean pressure (SW.MeanP_{Corr}) in addition to the measured mean pressure (SW.MeanP).

A first type of selectable thresholds 1807 relates to pressure ranges of dSW.x. A second type of selectable thresholds 1810 relates to pressure ranges of SW.x.PSL.PD of definable durations (SW.x.PSL.TD) calculated from the same type of single pressure wave (SW.x)-related parameters. A third type of selectable thresholds 1811 relates to ratios for combinations of pressure stability levels of different types of single pressure wave (SW.x)-related parameters.

Readings of the corrected mean pressure 1814 and any non-corrected mean pressure (meanP) are presented on a common pressure baseline.

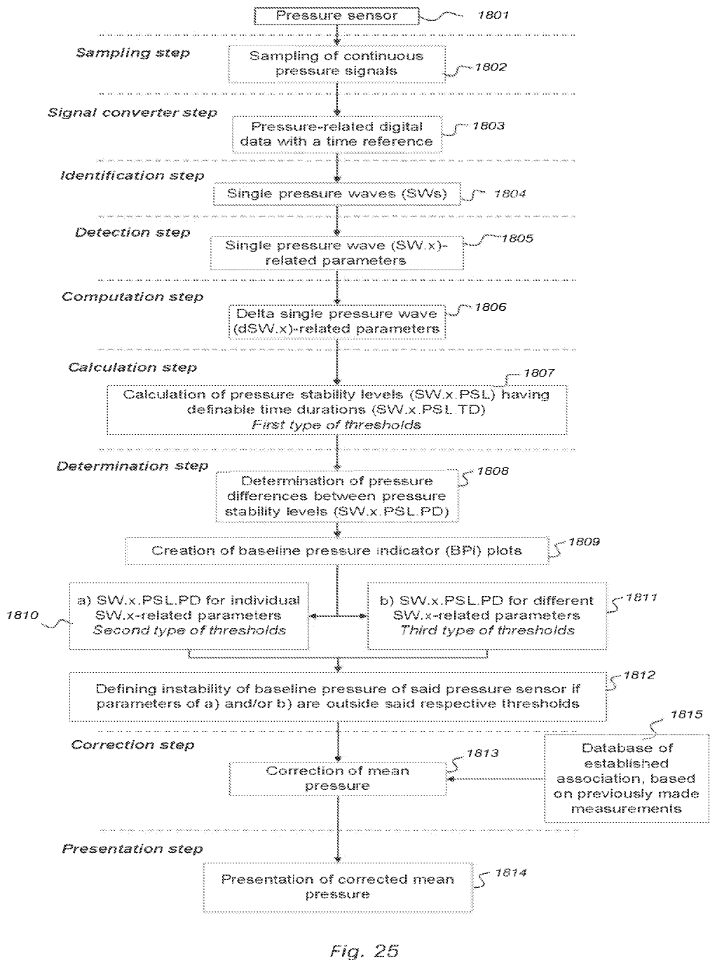

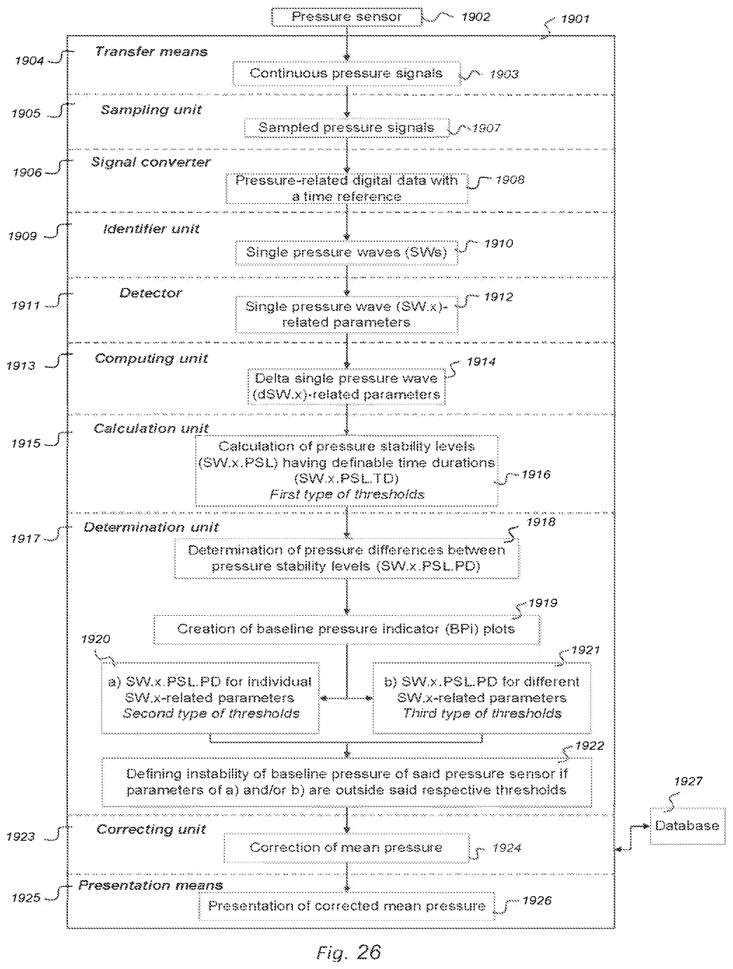

FIG. 26 illustrates a system for correcting mean pressure alterations caused by instability of baseline pressure of a pressure sensor applied for sampling of continuous pressure signals, according to embodiments of the present disclosure. In particular, FIG. 9 illustrates a system 1901 for correcting mean pressure alterations caused by instability of baseline pressure of a pressure sensor 1902 applied for sampling of continuous pressure signals 1903 originating from locations inside a human body or body cavity, samples of the pressure signals from the sensor being obtainable at specific intervals, and being convertible into pressure-related digital data with a time reference,

the system 1901 further comprising:
transfer means 1904 configured to transferring the pressure signals 1903 from the pressure sensor 1902 to a sampling unit 1905,
a signal converter 1906 in communication with the sampling unit 1905 and configured to perform conversion of sampled pressure signals 1907 into pressure-related digital data with a time reference 1908,
an identifier unit 1909 to receive the digital data 1908 from the signal converter 1906 and identify therefrom single pressure waves 1910 related to cardiac beat-induced pressure waves,
a detector 1911 coupled to an output of the identifier unit 1909 and configured to detect single pressure wave (SW.x)-related parameters 1912, being one or more of:

mean single wave pressure (SW.meanP), and

mean single wave amplitude (SW.dP), and
a computing device 1913 coupled to an output of the detector 1911 and configured to compute one or more delta single pressure wave (dSW.x)-related parameters 1914, representing differences in single pressure wave (dSW.x)-related parameters being one or more of change in mean pressure (dSW.meanP) and change in amplitude (dSW.dP) between a consecutive number of single pressure waves (n−1;n),
wherein a calculation unit 1915 is coupled to the computing device 1913 and configured to calculate pressure stability levels (SW.x.PSL) 1916, each pressure stability level being created from consecutive single pressure waves having any one of delta single pressure wave (dSW.x)-related parameters dSW.meanP and dSW.dP 1914 within a first type of selectable thresholds, the first type of thresholds referring to defined pressure ranges of any one of the parameters dSW.meanP and dSW.dP 1914, and wherein a pressure stability level 1916 refers to average of any one of the single pressure wave (SW.x)-related parameters SW.meanP and SW.dP 1112,
wherein a determination unit 1917 is coupled to the calculation unit 1915 and configured to determine pressure differences between different of the pressure stability levels (n−1; n) (SW.x.PSL.PD) 1918,
wherein the pressure stability levels (SW.x.PSL) 1916 of definable time durations (SW.x.PSL.TD) relating to the time duration of the pressure stability levels (SW.x.PSL) 1916 and with beginning pressure differences and ending pressure differences (SW.x.PSL.PD) 1918 for each pressure stability level (SW.x.PSL) together creating a baseline pressure indicator (BPi) plot 1919, the beginning pressure difference being defined as a difference between a present pressure stability level and a previous pressure stability level and the ending pressure difference being defined as a difference between a present pressure stability level and a next pressure stability level,
wherein information about stability of baseline pressure of the pressure sensor being a function of at least one of:

a) combinations of pressure differences between different of the pressure stability levels (SW.x.PSL) 1916 calculated from the same type of single pressure wave (SW.x)-related parameters, the pressure differences being outside or inside a second set of thresholds 1920, reflecting deviations from nominal reference pressure differences, and

b) relationships between different and simultaneous pressure stability levels (n−1; n) calculated from different types of single pressure wave (SW.x)-related parameters 1914, the relationships being outside or inside a third set of thresholds 1921, reflecting deviations from nominal reference relationships, and

wherein parameters of a) and/or b) outside the second set and/or the third set of thresholds define instability of baseline pressure of the pressure sensor 1922, and
wherein a mean pressure correcting unit 1923 is coupled to the determination unit 1917 and configured to correct mean pressure (SW.meanP) levels 1924 related to baseline pressure instability as a function of the pressure differences between different of the pressure stability levels (SW.x.PSL.PD) 1918, the corrections 1924 being selectable according to defined criteria, and

wherein a presentation means 1925 is coupled to the mean pressure correcting unit 1923 for presenting corrected mean pressure 1926.

Aspects of the disclosure may also relate to a system 1901 for measurement of ICP from an implantable pressure sensor 1902 incorporating methodology for correction of mean ICP errors caused by baseline pressure instability. Implantation of a miniature ICP sensor 1902 epidural may be able to detect ICP pulse waves, while baseline pressure variability may prevent determination of static ICP (mean ICP).

The body cavity may be a cranio-spinal cavity providing for measurement of intracranial pressure (ICP) or a blood-vessel compartment providing for measurement of arterial blood pressure (ABP). The pressure sensor 1902 may be configured for measuring of ICP or ABP signals and may be implantable for a shortened or temporary period or for an extended period of weeks or months. Pressure signals 1903 obtainable by the pressure sensor 1902 are transferred wireless when using sensor implantable for the extended period.

The first set of thresholds 1916 relate to pressure ranges of dSW.x 1914. The set type of thresholds 1920 relate to pressure ranges of SW.x.PSL.PD 1918 of definable durations (SW.x.PSL.TD) of the same type of single pressure wave (SW.x)-related parameters 1912. The third set of thresholds 1921 relates to ratios for combinations of pressure stability levels of different types of single pressure wave (SW.x)-related parameters 1912.

Regarding single wave parameters 1912, mean pressure (SW.meanP) detected by the detector represents a static pressure being mean single wave pressure (SW.meanP) relative to a baseline pressure being atmospheric pressure. With reference to FIGS. 7a-b, mean pressure (SW.meanP) 204, 208 represents average of static pressure samples divided by number of samples either during a rise time phase of the single pressure wave 209 or during an entire wave duration 210 of the single pressure wave. The amplitude (SW.dP) 211 detected by the detector represents difference in pressure between systolic maximum 206 and diastolic minimum pressures 205.

Further, a change in mean pressure (dSW.meanP) 1914 computed by the computing device 1913 is between a selectable number of single pressure waves (n−1;n), and represents change in absolute pressure between the single pressure waves. The change in amplitude (dSW.dP) 1914 computed by the computing device 1913 can be between a selectable number of single pressure waves (n−1;n), and represents change in internal signal relative pressure between the single pressure waves.

The change in mean pressure (dSW.meanP) 1914 determined by the computing device 1913 of the system 1901 may be a function of change in one or more of amplitude (dSW.dP), rise time (dSW.RT) and rise time coefficient (dSW.RTC), and may be related to database information 1917 about frequency of occurrence of expected differences.

The pressure stability level 1916 refer to average value of either of the single pressure wave parameters SW.meanP and SW.dP 1912, and wherein the single pressure waves 1910 included in the pressure stability level 1916 are based on either of the respective delta single pressure wave parameters 1914 dSW.meanP and dSW.dP, the parameters being within a first type of selectable thresholds. The pressure differences between pressure stability levels 1918 refer to difference in average pressure of the pressure stability levels 1916. Nearby pressure stability levels 1916 are merged into one pressure stability level if pressure difference (between pressure stability levels is within a second type of selectable thresholds. Therefore, information about stability of baseline pressure incorporates: a) information of pressure difference between different pressure stability levels 1918 of a definable number of the single wave parameter SW.meanP, and b) comparison of pressure stability levels 1916 of different single wave parameter, such as single wave parameters are SW.meanP and SW.dP 1912.

The mean pressure (SW.MeanP) is corrected 1924 by subtracting the mean pressure (SW.MeanP) level of a selectable number of single pressure waves of a pressure stability level by the first pressure difference of the pressure stability level. The correction of mean pressure 1924 may be presented 1926 as descriptive information such as corrected mean pressure (SW.MeanP_{Corr}) in addition to the measured mean pressure (SW.MeanP).

The readings of the corrected mean pressure 1926 and any non-corrected mean pressure (meanP) are presented on a common pressure baseline.

Using a database 1927 of information coupled to the system 1901, correction of mean pressure 1924 may be aided.

Baseline pressure variability of a definable magnitude may be a function of technical flaws or functional instability of the pressure sensor 1902, and being defined by changes in mean pressure (dSW.meanP) versus change in amplitude (dSW.dP) outside selectable ranges and thresholds of differences in mean pressure versus amplitude.

Associations between the single pressure wave (SW.x)-related parameters 1912 mean pressure (SW.meanP) and amplitude (SW.dP) may be established from previously established measurements, and is stored in a database 1927. Hence, the association may be determined by tabular presentations or by determining distribution based on measurements data deliverable from the database. Associations outside the second and/or third types of selectable set thresholds reflect deviations from nominal baseline pressure, the deviations being denoted as “baseline pressure errors”. Further, a time stamp and degree/amount of the deviations is determined by the parameter change defining unit.

Associations between the delta single pressure wave (dSW.x)-related parameters change in mean pressure (dSW.meanP) and change in amplitude (dSW.dP) may be created from previously established measurements, and is stored in a database. The variability of baseline pressure may be a change in mean pressure (dSW.meanP) versus change in amplitude (dSW.dP) above selectable thresholds.

The correction of mean pressure is presented 1926 as descriptive information such as corrected mean pressure (SW.MeanP_{Corr}) in addition to the measured mean pressure (SW.MeanP).

One example of correction of mean pressure derived from the test software is shown in FIG. 27a-b. FIGS. 27a-b illustrate the correction of mean intracranial pressure (ICP.SW.meanP) from the baseline pressure indicator (BPi) plot, according to embodiments of the present disclosure. FIG. 27a shows the trend plot of non-corrected mean intracranial pressure (ICP.SW.meanP), while FIG. 27b shows the trend plot of corrected mean intracranial pressure (ICP.SW.meanP_{CORR}) in addition to the trend plot of non-corrected mean intracranial pressure (ICP.SW.meanP).

The intracranial pressure is plotted on the y axis 2001 and time on the x axis 2002. The trend plots of SW.meanP 2003, 2004 and SW.dP 2005, 2006 is shown before and after a baseline pressure shift occurring at a specific time 2007, respectively. The baseline pressure indicator plot of SW.meanP 2008 is created from the pressure stability level of mean pressure (SW.meanP.PSL) before 2009 and after 2010 the baseline pressure shift 2007, as well as the pressure difference 2011 between the pressure stability levels 2009, 2010. In addition, the baseline pressure indicator plot for SW.dP 2012 incorporates the pressure stability level before 2013 and after 2014 the time point of the shift 2007. In FIG. 20a, the SW.meanP 2004 after time 2007 is uncorrected, while in FIG. 20b the SW.meanP is corrected (SW.MeanP_{Corr}) 2015. For this purpose, the mean pressure (SW.meanP) 2004 of every single wave is corrected according to the pressure difference 2011 between pressure stability level before 2009 and after 2010 the shift 2007. Hence, both the pressure stability level (SW.x.PSL) and pressure difference (SW.x.PSL.PD) represent input for adjustment of mean pressure

When different pressures are measured simultaneously from different pressure sensors, it may be determined how single pressure wave parameters (SW.x) correlate. A commonly used approach is determination of correlation between pressure parameters. Some examples of determination of correlation between pressure parameters are: pressure reactivity index (PRx) being the moving correlation between ICP.SW.meanP and ABP. SW.meanP, or the relationship amplitude-pressure (RAP) is the correlation between ICP.SW.meanP and ICP.SW.dP. A third correlation is the ICP-ABP amplitude correlation (IAAC) being the moving correlation between ICP.SW.dP and ABP.SW.dP. In this description the term correlation is used to denote association or how single wave parameters co-variate.

Concerning the pressure-reactive index (PRx), it is a moving correlation between the static pressure parameters mean ICP and mean ABP. When the Pearson correlation approaches +1, the correlation between mean ICP and mean ABP is high, which may be interpreted as loss of cerebrovascular auto-regulation. Instability of baseline pressure, either of mean ABP or mean ICP, impacts the interpretation of the PRx.

The parameter “Relationship Amplitude Pressure” (RAP) represents the moving correlation between mean ICP and the ICP amplitude, and is interpreted to contain information about intracranial compliance. A change in baseline (or reference) pressure will alter the mean ICP and causing the RAP to become erroneous.

A moving correlation between the ICP and ABP single wave parameter amplitudes has been denoted the IAAC, which may be interpreted as indicative of the cerebrovascular pressure regulation. This latter parameter is not affected by baseline pressure instability.

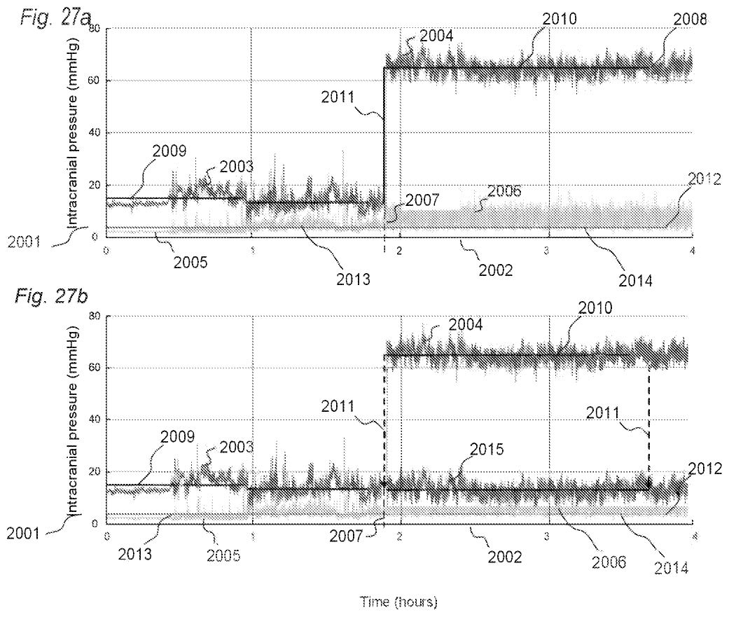

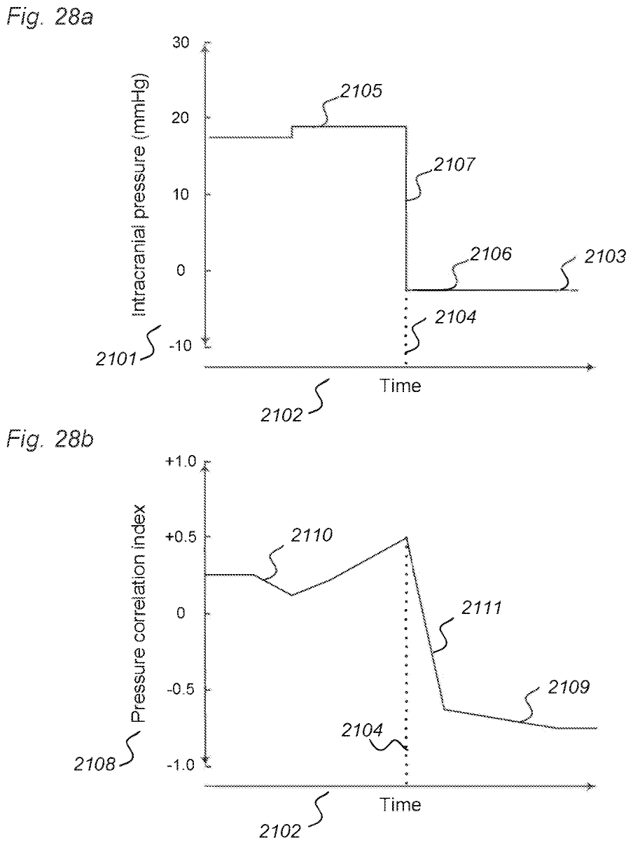

How, the present disclosure may be presented during monitoring of different pressures (such as CPP) or a pressure correlation index (such as PRx, RAP and IAAC) is schematically illustrated in FIG. 28a-b. FIGS. 28a-b illustrate the combined plotting over time of (FIG. 28a) baseline pressure indicator (BPi) plot and (FIG. 28b) pressure correlation index, according to embodiments of the present disclosure.

In FIG. 28a, intracranial pressure is presented on the y axis 2101 and time 2102 on the x-axis, showing a baseline pressure indicator plot 2103 for mean pressure (SW.meanP) is presented. At time 2104, there is a marked change in the plot. The baseline pressure indicator plot 2103 is created from the pressure stability levels (SW.meanP.PSL) before 2105 and after 2106 the time 2104, and the pressure difference (SW.meanP.PSL.PD) 2107 between the pressure stability levels 2105 and 2106. FIG. 28b shows on the y-axis the correlation coefficient 2108, and time along the x-axis 2102. The plot 2109 shows the moving correlation coefficient between mean pressure of ICP (ICP.SW.meanP) and ABP (ABP.SW.meanP), denoted pressure-reactivity index, or PRx. The PRx plot before 2110 and after 2111 the shift 2004 is different. By combining information from such plots, information is given for reasons to sudden changes in correlation coefficients.

In some embodiments, baseline pressure indicator plot may be used for correction of both mean pressure and pressure correlation.

Aspects of the disclosure include tools for assessing baseline pressure instability and correlation (co-variation), and issuance of alert when baseline pressure instability and correlation exceeds set thresholds. For example, this step may be applied to measurements of the so-called PRx (i.e. statistical moving Pearson correlation between mean ICP and mean ABP). In some embodiments, means are made available for detection of both baseline pressure instability and pressure correlation. Thereby, baseline pressure instability affecting pressure correlation may be detected, and alerts may be issued when baseline pressure instability and/or pressure correlation exceeds set thresholds.

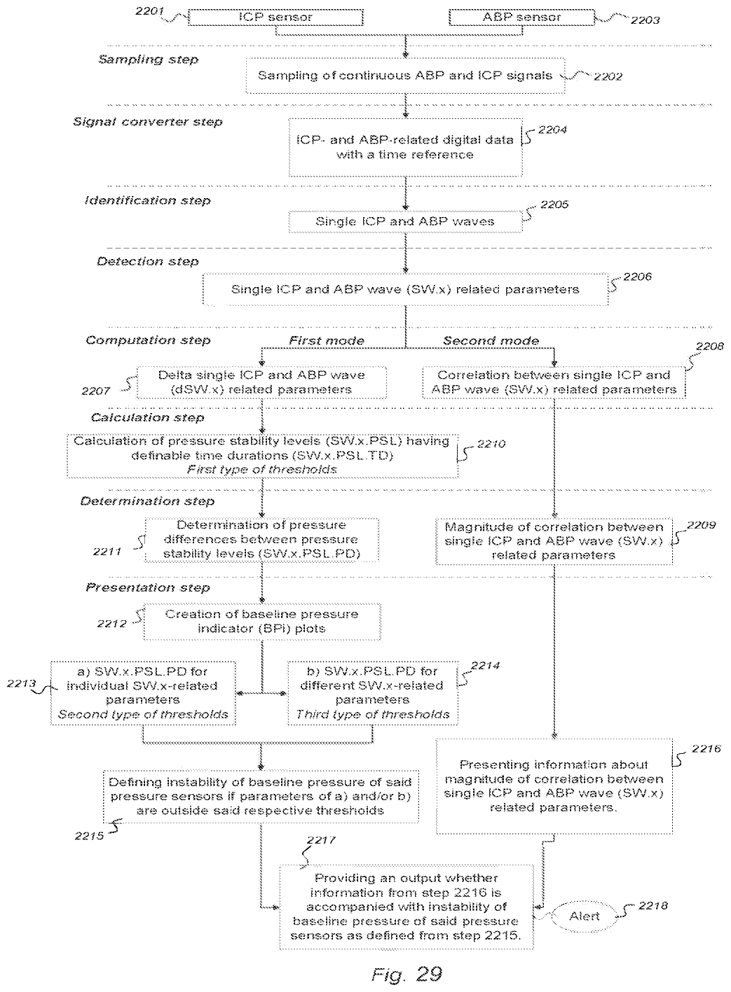

FIG. 29 illustrates a method for assessing information about stability of baseline pressure and pressure correlation of at least one intracranial pressure (ICP) sensor applied for sampling of continuous ICP signals originating from inside a cranio-spinal cavity and at least one arterial blood pressure (ABP) sensor applied for sampling of continuous ABP signals originating from inside a blood-vessel compartment, according to embodiments of the present disclosure.

In particular, FIG. 29 illustrates a method for assessing information about stability of baseline pressure and pressure correlation of at least one intracranial pressure (ICP) sensor 2201 applied for sampling of continuous ICP signals 2202 originating from inside a cranio-spinal cavity and at least one arterial blood pressure (ABP) sensor 2203 applied for sampling of continuous ABP signals 2202 originating from inside a blood-vessel compartment,

samples of the ICP and ABP signals 2202 from the ICP and ABP sensors 2201, 2203 being obtainable at specific intervals, and being convertible into pressure-related digital data with a time reference 2204,

the method comprising:

identifying from the digital data of the ICP 2201 and ABP 2203 sensors single ICP and ABP waves 2205 related to cardiac beat-induced pressure waves,

and for each of the ICP and ABP signals 2202:

detection from the digital data 2204 of single pressure wave (SW.x)-related parameters 2206 selectable from one or more of mean pressure (SW.meanP) and amplitude (SW.dP),

in a first mode:

based on the detection, computation of delta single pressure wave (dSW.x)-related parameters 2207 between a selectable number of single pressure waves (n−1;n), representing differences in single pressure wave (dSW.x)-related parameters selectable from one or more of change in mean pressure (dSW.meanP) and change in amplitude (dSW.dP) between a selectable number of single pressure waves (n−1;n),

and

in a second mode:

from the digital data 2204, computation of correlation between one or more of the single pressure wave (SW.x)-related parameters 2208 selected from one or more of mean pressure (SW.meanP) and amplitude (SW.dP) of the ICP and ABP sensors,

and determination of magnitude of correlation 2209 between single pressure wave parameters 2206 of the ICP 2201 and ABP 2203 sensors,

wherein further in the first mode:

calculation of pressure stability levels (SW.x.PSL) 2210 each pressure stability level being created from consecutive single pressure waves having any one of delta single pressure wave (dSW.x)-related parameters dSW.meanP and dSW.dP 2207 within a first type of selectable thresholds, the thresholds referring to defined pressure ranges of any one of the parameters dSW.meanP and dSW.dP 2207, and wherein a pressure stability level refers to average of any one of the single pressure wave (SW.x)-related parameters SW.meanP and SW.dP 2206, and

determination of pressure differences between different of the pressure stability levels (n−1;n) (SW.x.PSL.PD) 2211,

and

creation of baseline pressure indicator (BPi) plots 2212 using the pressure stability levels (SW.x.PSL) 2210 of definable time durations (SW.x.PSL.TD) relating to the time duration of the pressure stability levels (SW.x.PSL) and with beginning pressure differences and ending pressure differences (SW.x.PSL.PD) 2211 for each pressure stability level (SW.x.PSL), the beginning pressure difference being defined as a difference between a present pressure stability level and a previous pressure stability level and the ending pressure difference being defined as a difference between a present pressure stability level and a next pressure stability level,

the plots 2212 providing information about stability of baseline pressure of the pressure sensor and being a function of at least one of:

a) combinations of pressure differences between different of the pressure stability levels calculated from the same type of single pressure wave (SW.x)-related parameters, the pressure differences being outside or inside a second set of thresholds 2213, reflecting deviations from nominal reference pressure differences, and

b) relationships between different and simultaneous pressure stability levels (n−1; n) calculated from different types of single pressure wave (SW.x)-related parameters, the relationships being outside or inside a third set of thresholds 2214, reflecting deviations from nominal reference relationships, and

wherein parameters of a) and/or b) outside the second set and/or the third set of thresholds define instability of baseline pressure of the pressure sensor 2215,

wherein further in the second mode:

presentation of information about magnitude of correlation between single ICP and ABP wave (SW.x) related parameters 2216, and

wherein an output 2217 is given to indicate whether the information in the second mode about magnitude of correlation between single ICP and ABP wave related parameters 2216 is accompanied with baseline pressure instability as defined in 2215 of the first mode.

The ICP 2201 and ABP 2203 sensors are implantable for a shortened or temporary time period or for an extended period of weeks or months. The pressure signals 2202 obtainable by the pressure sensors 2201, 2203 are wireless transferred when the sensor is implanted for the extended period.

A pressure sensor 2201, 2203 used according to the method may be implanted for a shortened or temporary period or for an extended period of weeks or months. Pressure signals 2202 obtainable by the pressure sensor 2201, 2203 may be wireless transferred when the sensor 2201, 2203 is implanted for the extended period.

The pressure stability level 2210 refer to average value of any one of the single pressure wave parameters SW.meanP and SW.dP 2206, and wherein the single pressure waves 2205 included in the pressure stability level 2210 are based on any one of the respective delta single pressure wave parameters 2207 dSW.meanP and dSW.dP, the parameters 2207 being within selectable thresholds. The pressure differences between pressure stability levels (SW.x.PSL.PD) 2211 refer to difference in average pressure of the pressure stability levels 2210. Nearby pressure stability levels 2210 are merged into one pressure stability level if pressure difference between pressure stability levels 2211 is within selectable thresholds.

The information about stability of baseline pressure 2215 incorporates information of: a) pressure difference between different pressure stability levels of a selectable number of the single wave parameter SW.meanP 2213, and/or b) comparison of pressure stability levels of different single wave parameter, such as the single wave parameters are SW.meanP and SW.dP 2214.

Regarding single pressure wave parameters 2206, mean pressure (SW.meanP) 204, 208 represents a static pressure being mean single wave pressure (SW.meanP) relative to a baseline pressure being atmospheric pressure. Mean pressure (SW.meanP) may represent average of static pressure samples divided by number of samples either during a rise time phase of the single pressure wave 209 or during an entire wave duration of the single pressure wave 210. Amplitude (SW.dP) 211 represents differences in pressure between systolic maximum 206 and diastolic minimum pressures 205.

Regarding delta single pressure wave parameters 2207, change in mean pressure (dSW.meanP) 213, 214 may be between a selectable number of single pressure waves 203 and represents change in absolute pressure between the single pressure waves. The change in amplitude (dSW.dP) 215, 216 may be between a selectable number of single pressure waves (n−1;n), and represents change in internal signal relative pressure between the single pressure waves.

Baseline pressure instability of a definable magnitude may be a function of technical flaws or functional instability of the pressure sensor 2201, 2203, and may be defined by changes in mean pressure versus change in amplitude outside selectable ranges and thresholds of differences in mean pressure versus amplitude.

Correlation between the single pressure wave (SW.x)-related parameters 2208 may be one or more of: mean pressure (SW.meanP) and amplitude (SW.dP) may be created from previously established measurements and stored in a database. The correlation may be determined by tabular presentations or by determining distribution based on measurements data deliverable from the database. Any of the associations outside selectable set thresholds may reflect deviations from a nominal baseline pressure, the deviations being denoted as “baseline pressure errors”. A time stamp and degree/amount of the deviations may be determined.

The output 2217 whether information from step 2216 about correlation between single ICP and ABP wave related parameters is accompanied with pressure sensor instability as defined in step 2215 also incorporates issuance of an alert 2218 from the comparison and presentation step 2217 in the presence of at least one of: a) the combinations of pressure differences between different of the pressure stability levels (SW.x.PSL) calculated from the same type of single pressure wave (SW.x)-related parameters, the pressure differences being outside a second type of selectable set thresholds 2213, reflecting deviations from nominal reference pressure differences, and b) relationships between different and simultaneous pressure stability levels (n−1; n) calculated from different types of single pressure wave (SW.x)-related parameters, the relationships being outside a third type of selectable set thresholds 2214, reflecting deviations from nominal reference relationships. The alert 2218 being at least one of: a warning color present on at least one part of the baseline indicator plot displayable in the comparison and presentation step 2217, a warning noise, and a visible or audible descriptive information.

A first type of selectable thresholds 2210 relates to pressure ranges of dSW.x. A second type of selectable thresholds 2213 relates to pressure ranges of SW.x.PSL.PD of definable durations (SW.x.PSL.TD) calculated from the same type of single pressure wave (SW.x)-related parameters, the pressure differences. A third type of selectable thresholds 2214 relates to ratios for combinations of pressure stability levels of different types of single pressure wave (SW.x)-related parameters.

The correlation 2209 according to the method may refer to moving correlation between mean ICP and mean ABP. Issuance of an alert 2218 may provide for information about magnitude of correlation 2216 and time of the correlation, and the alert 2218 being at least one of: a warning color on an output monitor display in the comparison and presentation step 2217, warning noise, and an visible or audible descriptive information output from the comparison and presentation step 2217.

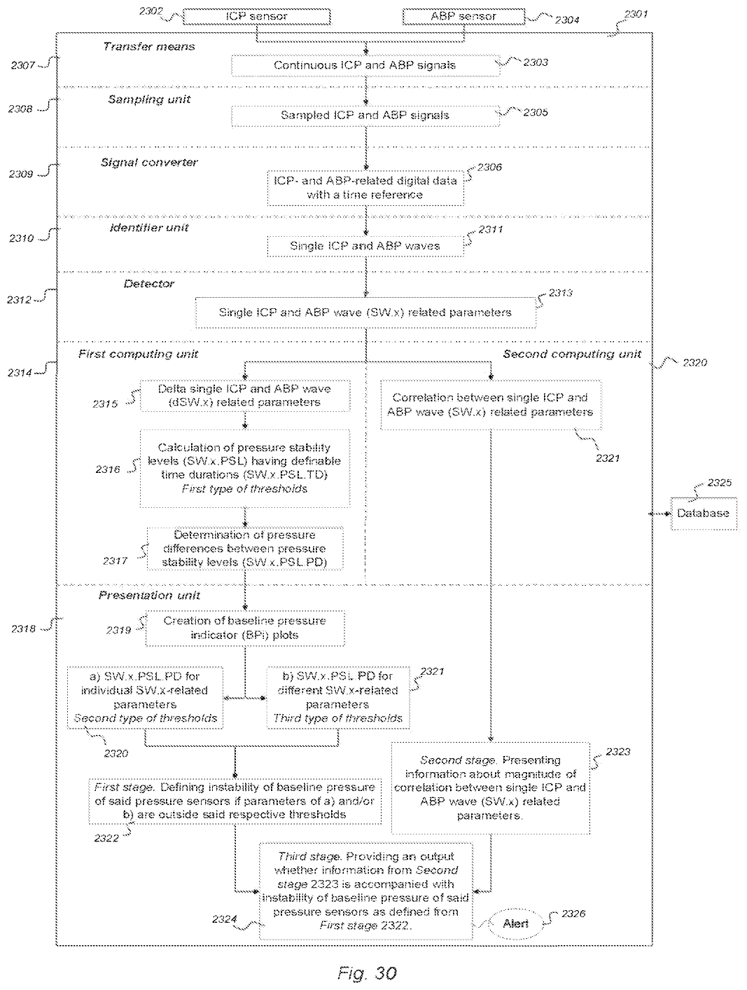

FIG. 30 illustrates a system for assessing information about stability of baseline pressure and pressure correlation of at least one intracranial pressure (ICP) sensor applied for sampling of continuous ICP signals originating from inside a cranio-spinal cavity and at least one arterial blood pressure (ABP) sensor applied for sampling of continuous ABP signals originating from inside a blood-vessel compartment, according to embodiments of the present disclosure.

In particular, FIG. 30 illustrates a

system 2301 for assessing information about stability of baseline pressure and pressure correlation of at least one intracranial pressure (ICP) sensor 2302 applied for sampling of continuous ICP signals 2303 originating from inside a cranio-spinal cavity and at least one arterial blood pressure (ABP) sensor 2304 applied for sampling of continuous ABP signals 2303 originating from inside a blood-vessel compartment,

samples of the ICP and ABP signals 2305 from the ICP 2302 and ABP 2304 sensors being obtainable at specific intervals, and being convertible into pressure-related digital data with a time reference 2306,

the system 2301 comprising:
transfer means 2307 being coupled to the ICP 2302 and ABP 2304 sensors and being configured to transfer the respective ICP and ABP signals 2303 to a sampling unit 2308,
a signal converter 2309 in communication with the sampling unit 2308 and configured to perform conversion of sampled ICP and ABP signals 2305 into pressure-related digital data with a time reference 2306,
an identifier unit 2310 to receive the pressure-related digital data 2306 from the signal converter 2309 and identify therefrom ICP and ABP single pressure waves 2311 related to cardiac beat-induced pressure waves,
a detector 2312 being coupled to the identifier unit 2310 and being configured to detect from the respective ICP and ABP single pressure waves 2311, single pressure wave (SW.x)-related parameters 2313, being one or more of single wave mean pressure (SW.meanP) and single wave amplitude (SW.dP),
a first computing device 2314 being coupled to the detector 2312 and configured for determination of stability of baseline pressure and configured to compute from the detected parameters of ICP and ABP single pressure waves 2313, delta single pressure wave (dSW.x)-related parameters 2315 between a selectable number of single pressure waves (n−1;n) 2311, representing differences in single pressure wave (SW.x)-related parameters, being one or more of change in mean pressure (dSW.meanP) and change in amplitude (dSW.dP)2315 between a consecutive number of single pressure waves (n−1;n) 2311,
a second computing device 2320 being coupled to the detector 2312 and configured for computation of correlation and magnitude of correlation between one or more of the single pressure wave (SW.x)-related parameters 2321 being one or more of: mean pressure (SW.meanP) and amplitude (SW.dP)) 2313 of the ICP 2302 and ABP 2304 sensors,

wherein the first computing device 2314 is further configured to:
in a calculation stage, calculate pressure stability levels (SW.x.PSL) 2316, each pressure stability level being created from consecutive single pressure waves having any one of delta single pressure wave (dSW.x)-related parameters dSW.meanP and dSW.dP within a first type of selectable thresholds 2316, the first type of thresholds referring to defined pressure ranges of any one of the parameters dSW.meanP and dSW.dP 2315, and wherein a pressure stability level 2316 refers to average of any one of the single pressure wave (SW.x)-related parameters SW.meanP and SW.dP 2313, and
in a determination stage 2317, determine pressure differences between different pressure stability levels (n−1; n) (SW.x.PSL.PD),
a presentation unit 2318 configured to create baseline pressure indicator (BPi) plots 2319 from pressure stability levels (SW.x.PSL) of definable time durations (SW.x.PSL.TD) 2316 and with beginning pressure differences and ending pressure differences (SW.x.PSL.PD) for each pressure stability level (SW.x.PSL) 2317,

wherein the BPi plots 2319 provide information about stability of baseline pressure of the pressure sensor and are a function of at least one of:

a) combinations of the pressure differences between different of the pressure stability levels calculated from the same type of single pressure wave (SW.x)-related parameters, the pressure differences being outside or inside a second set of thresholds 2320, reflecting deviations from nominal reference pressure differences, and

b) relationships between different and simultaneous pressure stability levels (n−1; n) calculated from different types of single pressure wave (SW.x)-related parameters, the relationships being outside or inside a third set of thresholds 2321, reflecting deviations from nominal reference relationships, and

wherein the presentation unit 2318 in a first stage parameters of a) and/or b) outside the second set and/or the third set of thresholds define instability of baseline pressure of the pressure sensor 2322,

wherein an output from the second computing device 2320; 2321 is connected to a second stage 2323 of the presentation unit 2318, the second stage 2323 configured to provide presentation of information about magnitude of correlation between single ICP and ABP wave (SW.x) related parameters,

and

wherein the presentation unit 2318 has a third stage 2324 connected to output from the first stage 2322 and second stage 2323, the third stage 2324 being configured for providing an output whether information from second stage 2323 is accompanied with baseline pressure instability as defined from first stage 2322.

The ICP 2302 and ABP 2304 sensors are implantable for a shortened or temporary time period or for an extended period of weeks or months. The transfer means 2307 is of wireless type when used for sensors 2302, 2304 implantable for the extended period.

The pressure sensor 2302, 2304 used by the system 2301 may be implantable for a shortened or temporary period or for an extended period of weeks or months. When pressure sensors 2302, 2304 are implanted for an extended period, transfer means may be of wireless type.

The pressure stability level 2316 refer to average value of either of the single pressure wave parameters SW.meanP and SW.dP 2313, and wherein the single pressure waves 2311 included in the pressure stability level 2316 are based on either of the respective delta single pressure wave parameters dSW.meanP and dSW.dP 2315, the parameters being within the first type of selectable thresholds. The pressure differences between pressure stability levels 2317 refer to difference in average pressure of the pressure stability levels 2316. Nearby pressure stability levels 2316 are merged into one pressure stability level 2316 if pressure difference (between pressure stability levels 2317 is within selectable thresholds. Accordingly, information about stability of baseline pressure 2319 incorporates: a) information of pressure difference between different pressure stability levels 2320 of a selectable number of the single wave parameter SW.meanP, and b) information from comparing of pressure stability levels 2316 of different single wave parameter 2321, such as the single wave parameters SW.meanP and SW.dP.

Regarding single pressure waves 2311, mean pressure (SW.meanP) represents a static pressure being mean single wave pressure (SW.meanP) relative to a baseline pressure being atmospheric pressure. The mean pressure (SW.meanP) may represent average of static pressure samples divided by number of samples either during a rise time phase of the single pressure wave or during an entire wave duration of the single pressure wave. The amplitude (SW.dP) represents differences in pressure between systolic maximum and diastolic minimum pressures.

Regarding delta single pressure wave parameters 2315, change in mean pressure (dSW.meanP) 213, 214 may be between a definable number of single pressure waves 203 and represents change in absolute pressure between the single pressure waves. Change in amplitude (dSW.dP) 215, 216 between a definable number of single pressure waves 203 may represent change in internal signal relative pressure between the single pressure waves.

According to the system 2301, baseline pressure instability of a definable magnitude may be a function of technical flaws or functional instability of the pressure sensor 2302, 2304, and being defined by changes in mean pressure versus change in amplitude outside selectable ranges and thresholds of differences in mean pressure versus amplitude.

Correlation between the single pressure wave (SW.x)-related parameters 2321 mean pressure (SW.meanP) and amplitude (SW.dP) may be created from previously established measurements and stored in a database 2325. The association may be determined by tabular presentations or by determining distribution based on measurements data deliverable from the database. Moreover, association between the delta single pressure wave (dSW.x)-related parameters 2315 change in mean pressure (dSW.meanP) and change in amplitude (dSW.dP) may be created from previously established measurements and stored in a database 2325.

Any of the associations outside selectable set thresholds may reflect deviations from a nominal baseline pressure, the deviations being denoted as “baseline pressure errors”.

The information about stability of baseline pressure from the first stage 2322 incorporates issuance from the third stage 2324 of an alert 2326 in the presence of at least one of:

a) the combinations of pressure differences between different pressure stability levels calculated from the same type of single pressure wave (SW.x)-related parameters, the pressure differences being outside a second set of thresholds 2320, reflecting deviations from nominal reference pressure differences, and

b) relationships between different and simultaneous pressure stability levels (n−1; n) calculated from different types of single pressure wave (SW.x)-related parameters, the relationships being outside a third set of thresholds 2321, reflecting deviations from nominal reference relationships. The alert 2326 being delivered by the third stage 2324 and being at least one of: a warning color on at least one part of the baseline pressure indicator plot shown on an output monitor screen of the third stage 2324, a warning noise, and a visible or audible descriptive information.

An alert 2326 delivered by the third stage 2324 and being at least one of: a warning color on at least one part of the baseline pressure indicator plot shown on an output monitor screen of the third stage 2324, a warning noise, and a visible or audible descriptive information. The thresholds of the a) pressure difference between different pressure stability level 2317 and thresholds of the b) relationship between different pressure stability levels 2316 are created from previously established measurements and stored in a database.

The correlation 2321 may refer to moving correlation between mean ICP and mean ABP. An alert 2326 may be indicative of correlation margins and time of their occurrence. Issuance of an alert 2326 may provide for information about magnitude of correlation and time of the correlation 2321 made in the second computing device 2320, and wherein the alert 2326 as provided by the third stage 2324 being at least one of: a warning color on an output monitor screen of the third stage 2324, a warning noise, and a visible or audible descriptive information.

A second set of thresholds of the a) combinations of pressure differences between different of the pressure stability levels (n−1; n) (SW.x.PSL), and the third set of thresholds of the b) relationships between different and simultaneous pressure stability levels (n−1; n) calculated from different types of single pressure wave (SW.x)-related parameters are created from previously established measurements and stored in a database 2325. A first set of thresholds relate to pressure ranges of dSW.x, a second type of selectable thresholds relate to pressure ranges of SW.x.PSL.PD of definable durations (SW.x.PSL.TD) calculated from the same type of single pressure wave (SW.x)-related parameters, the pressure differences, and a third set of thresholds relate to ratios for combinations of pressure stability levels of different types of single pressure wave (SW.x)-related parameters.

Aspects of the disclosure provide solutions on how to provide surveillance of baseline pressure instability from multiple pressure sensors and pressure transducer systems at the same time. Currently, there are no described solutions for remote and centralized surveillance of baseline pressure instability from many pressure sensors. Remote processing of continuous pressure monitoring has many advantages related to creating data availability, sharing of data, and, in some contexts, utilizing big data for optimization of assessment of baseline pressure instability. Furthermore, there may be a substantial cost-saving of having a remote processing with addressed feedbacks, collection of statistical data, reporting of status of sensor instability, and causing alerts if sensor instability is critical to patient safety. Today, users of sensors, such as hospital environments, and manufacturers of pressure sensors and pressure transducer systems, might not have an opportunity for surveillance and monitoring of pressure sensors to identify the occurrence of pressure sensor failure leading to baseline pressure instability. Moreover, physicians or research groups utilizing continuous pressure measurements in humans might not have the means for surveillance and alerts of baseline pressure instability. Such information would affect the usefulness of information provided by the pressure sensors.

Baseline pressure instability represents a risk of hazard to patients. Malfunction of pressure sensors and pressure transducer systems may have many causes, such as technical malfunction. For manufacturers of pressure sensors, it is essential to exclude product malfunction. Today, the manufacturers have limited means for continuous surveillance of their products. If the end-user suspects malfunction of pressure equipment during pressure monitoring, the sensor has to be removed from the patient and sent to the laboratory for testing. However, the behavior of the sensor may differ substantially whether it is implanted in a patient or after removal. The conventional strategy is to test whether pressures show zero value in atmospheric pressure before and after pressure recording to exclude drift of zero pressure. This may be both time-consuming and difficult to verify whether or not a pressure sensor is failing. Verification of whether or not several sensors are failing may be very difficult. Aspects of the present disclosure enables continuous surveillance of pressure sensor function, notably while the sensor is measuring within the patient. The sensor does not need to be removed to be tested by the manufacturer. Instead, the user may provide remote processing of ongoing pressure measurements that may be accessed by the manufacturer to assess sensor failure. In other words, the present disclosure enables in vivo testing of the pressure sensor, as compared to current practices of bench testing of the pressure sensor after its removal from the human body. The remote processing of pressure signals may be accompanied by information about sensor number and batch number. Thereby, the manufacturer may check whether a certain batch of sensors is undergoing sensor failure. In cases of sensor failure, quick supply of replacement of faulty sensors can be improved, thereby enhancing patient safety. In some cases, baseline pressure errors may occur rather frequently. Failure may be in the pressure sensor or within the pressure transducer system. Tentatively, there may be periodic failures, or failures occurring after some use of the equipment. Therefore, there is a need for surveillance of this safety aspect of pressure sensors.

ICP monitoring has been used clinically over about 60 years, though its usefulness is still heavily debated. For example, it is debated whether ICP monitoring is improving outcome after traumatic brain injury even though traumatic brain injury is the main indication for ICP monitoring. Hence, clinical neuroscientists are performing research studies on the usefulness of ICP monitoring. Today, there are no opportunities for surveillance of baseline pressure instability during ICP monitoring. Aspects of the present disclosure enable remote analysis of ongoing ICP recordings included in a research study, thereby introducing an essential quality measure that improves the quality of research studies.

Aspects of the present disclosure describe remote processing of de-identified pressure-related digital data, which may also provide correction of pressures of different pressure sensors and pressure transducer systems. The corrected mean pressure values may be provided to the system as output to end users.

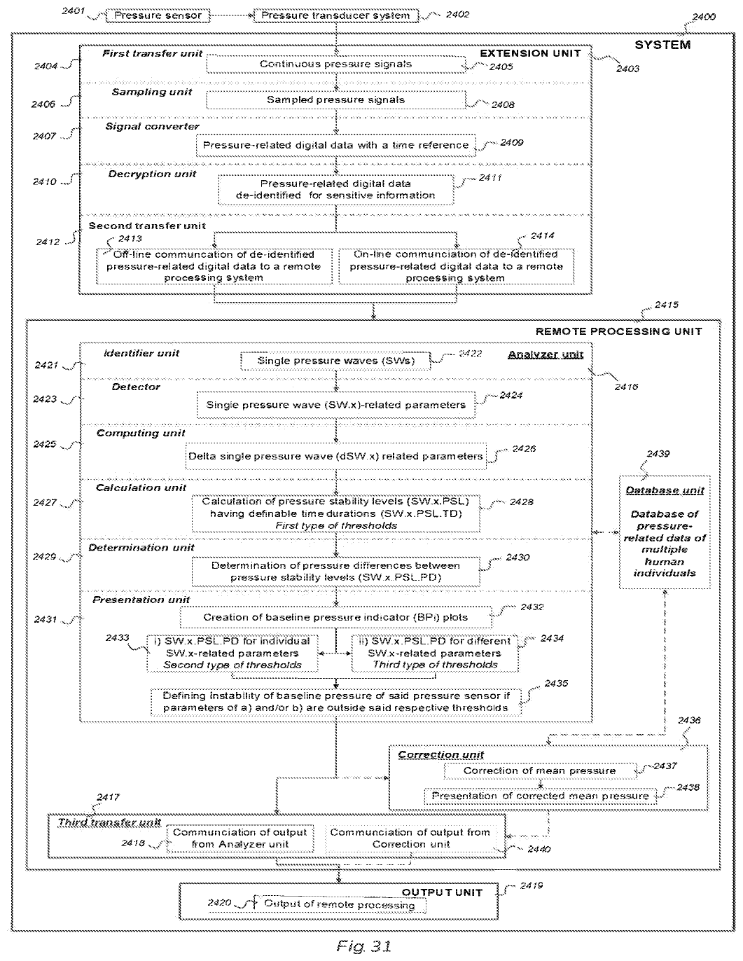

FIG. 31 illustrates a system 2400 for assessing stability of baseline pressure of a pressure sensor 2401 being in communication with a pressure transducer system 2402 and capable of measuring continuous pressure signals from inside a human body or body cavity.

The system 2400 comprises:

a) an extension unit 2403 comprising
a first transfer unit 2404 configured to transfer continuous pressure signals 2405 from the pressure sensor 2401 to a sampling unit 2406,
a signal converter 2407 in communication with the sampling unit 2406 and configured to perform conversion of sampled continuous pressure signals 2408 into pressure-related digital data with a time reference 2409,
a decryption unit 2410 configured to de-identify the pressure-related digital data with a time reference 2409 for sensitive information, resulting in pressure-related digital data de-identified for sensitive information 2411, and
a second transfer unit 2412 enabling for off-line 2413 or on-line 2414 communication of data de-identified for sensitive information 2411 to a remote processing unit 2415.

The system 2400 further comprises:

b) the remote processing unit 2415 comprising
an analyzer unit 2416 configured to analyze the pressure-related digital data de-identified for sensitive information 2411, and
a third transfer unit 2417 configured to transmit an analysis output 2418 from the analyzer unit 2416 to an output unit 2419.

The system 2400 also consists of:

c) the output unit 2419 configured to provide an output 2420 of the remote processing unit, wherein the output 2420 presents baseline pressure instability of a pressure sensor 2401.

The analyzer unit 2416 of the remote processing unit 2415 further comprises:
an identifier unit 2421 to receive the de-identified pressure-related digital data 2411 from the extension unit 2403 and the second transfer unit 2412, and identify, from the de-identified pressure-related digital data 2411, single pressure waves (SWs) 2422 related to cardiac beat-induced pressure waves,
a detector 2423 connected to an output of the identifier unit 2421 and configured to detect single pressure wave (SW.x)-related parameters 2424 from the single pressure waves, being at least one or more of mean pressure (SW.meanP), and amplitude (SW.dP), and
a computing unit 2425 connected to an output of the detector 2423 and configured to compute one or more of delta single pressure wave (dSW.x)-related parameters 2426 representing differences in single pressure wave (dSW.x)-related parameters being one or more of change in mean pressure (dSW.meanP), and change in amplitude (dSW.dP), between a consecutive number (n−1;n) of single pressure waves (SW.x) 2422.

In some embodiments, the computing unit 2425 may be referred to herein as a computing device. A calculation unit 2427 is connected to an output of the computing unit 2425 and configured to calculate pressure stability levels (SW.x.PSL) 2428, each pressure stability level being created from consecutive single pressure waves 2422 having any one of the delta single pressure wave (dSW.x)-related parameters dSW.meanP and dSW.dP 2426 within a first set of thresholds 2428, the first set of thresholds referring to defined pressure ranges of anyone of the parameters dSW.meanP and dSW.dP 2426, and wherein a calculated pressure stability level 2428 refers to an average of any one of the single pressure wave (SW.x)-related parameters SW.meanP and SW.dP 2424.

A determination unit 2429 is connected to an output of the calculation unit 2427 and configured to determine pressure differences (SW.x.PSL.PD) 2430 between different calculated pressure stability levels (n−1;n) (SW.x.PSL) 2428, having definable time durations (SW.x.PSL.TD) relating to the time duration of the calculated pressure stability levels (SW.x.PSL) 2428.

A presentation unit 2431 is connected to an output of the determination unit 2429 and configured to present baseline pressure indicator (BPi) plots 2432, being created from calculated pressure stability levels (SW.x.PSL) 2428 and with beginning pressure differences and ending pressure differences (SW.x.PSL.PD) 2430 for each pressure stability level (SW.x.PSL) 2428, the beginning pressure difference being defined as a difference between a present pressure stability level and a previous pressure stability level and the ending pressure difference being defined as the difference between a present pressure stability level and a next pressure stability level.

The baseline pressure indicator (BPi) plots 2432 provide information about stability of baseline pressure of the pressure sensor 2401 and are a function of at least one of:

i) combinations of the pressure differences between different of the pressure stability levels (SW.x.PSL) 2428, calculated from a same type of single pressure wave (SW.x)-related parameters 2424, the pressure differences being outside or inside a second set of thresholds 2433, reflecting deviations from nominal reference pressure differences, and

ii) relationships between different and simultaneous pressure stability levels (n−1; n) 2428 calculated from different types of single pressure wave (SW.x)-related parameters 2424, the relationships being outside or inside a third set of thresholds 2434, reflecting deviations from nominal reference relationships.

The presentation unit 2431 is configured to indicate if parameters of i) 2433 and/or ii) 2434 are outside the respective second set and/or the third set of thresholds and thereby define instability of baseline pressure of the pressure sensor 2435.

The system 2400 incorporates a mean pressure correction unit 2436 connected to an output of the analyzer unit 2416 of the remote processing unit 2415. The mean pressure correcting unit 2436 may be configured to correct the single pressure wave parameter mean pressure (SW.meanP) 2423 related to baseline pressure instability as a function of the pressure differences between different of the pressure stability levels (SW.x.PSL.PD) 2430. The correction of mean pressure 2437 is selectable according to defined criteria, and the correction unit 2436 provides for a presentation of corrected mean pressure 2438. Notably, the primary function of the system is to provide information about any instability of baseline pressure of pressure sensors. In some embodiments, this may be done without providing information about corrected mean pressures. Hence, in some embodiments, information about corrected mean pressures may be optional. Information that is of most interest may depend on an end user, and this may be customized based on the end user's needs.

One example of criteria for correction of mean pressure (SW.meanP) is given although this is not intended to limit the disclosure. With reference to FIG. 20a-b, the pressure stability level of mean pressure (SW.meanP.PSL) before 2009 and after 2010 the occurrence of a baseline pressure shift 2007 is shown. The pressure difference (SW.meanP.PSL.PD) 2011 between the pressure stability levels 2009, 2010 is >40 mmHg, are thereby outside the thresholds. Moreover, the relationship between SW.meanP.PSL and SW.dP.PSL was >10 and thereby outside the thresholds.

The extension unit 2403 of the system 2400 is capable of sampling continuous pressure signals 2405 from commercial pressure sensor transducer systems and vital signs monitors for use in human subjects. Examples of commercial pressure sensor transducer systems are shown in Table 17. In some embodiments, one extension unit 2403 is connected via a communication port to a pressure transducer system or a vital signs monitor 2402.

TABLE 17
Examples of pressure transducer systems 2402 that may be connected to
an extension unit 2403 for remote processing
utilizing the presently described system 2400.

| Pressure sensor 2401 | ICP transducer systems 2402 | Manufacturer |
|---|---|---|
| CereLink ICP sensor | CereLink ICP monitor | Integra LifeSciences, |
| | | USA |
| Sophysa Pressio ICP | Pressio ICP | Sophysa, France |
| sensor | | |
| Raumedic NeuroVent P | Raumedic ICP Monitor MPR1/2 | Raumedic, Germany |
| /NeuroDur sensor | | |
| Camino ICP | Natus Camino ICP Monitor | Natus Medical, USA |

Vital signs monitors

| | | |
|---|---|---|
| Multiple types of | PHILIPS Intellivue | Philips Healthcare, |
| pressure sensors | | Netherlands |
| | NIHON KODEN | |
| | DRAGER / SIEMENS Infinity | Draeger Medical, |
| | | Germany |
| | GE Dash / GE Datex Ohmeda | GE Healthcare, USA |
| | SPACELABS | Spacelabs Healthcare, |
| | | USA |
| | FUKUDA DENSHIDS-7000 | Fukuda Denshi, Japan |

Pressure sensors 2401 for measurements of continuous pressure signals 2405 in humans may incorporate a transducer 2402 in connection with the sensor 2401, built into a monitor system. As indicated in Table 17, pressure monitoring may be done either using dedicated pressure transducer systems 2402 or vital signs monitors 2402, or in some instances, the pressure transducer system is connected to a monitor. The pressure transducer systems 2402 or monitors 2402 may have communication protocols for external transfer of continuous signals from which an extension unit 2403 may be connected. The extension unit 2403 of the present disclosure may be connected to a pressure transducer system or a vital signs monitor, as detailed in Table 17.

With regard to decryption unit 2410 of the extension unit 2403, what is considered sensitive information may vary for different regions. In some embodiments, sensitive information may include information about birth data and social security codes, or information about data or time of a pressure recording, stored in the pressure-related digital data 2409. The de-identification of digital pressure-related digital data is performed prior to communication of data to a remote processing unit 2415.

The second transfer unit 2412 of the system 2400 is capable of wireless communication of the pressure-related digital data de-identified for sensitive information 2411 to the remote processing unit 2415. The remote processing unit 2415 may be related to a cloud-based software system or a dedicated stand-alone software system.

The third transfer unit 2417 enables for communication of output from the analyzer unit 2416 and from a correction unit 2436. The system 2400 may incorporate in the remote processing unit 2415 a database unit 2439, incorporating pressure-related digital data de-identified for the sensitive information 2411 from multiple human individuals. The third transfer unit 2417 may enable communication from the database 2439 via the analyzer unit 2416 to the output unit 2419. Moreover, the third transfer unit 2417 is able to communicate output of the correction unit 2436 to the output unit 2419. The third transfer unit 2417 is capable of wireless communication using commercial communication protocols. In some embodiments, it should be noted that wireless communication is not a requirement for the second and third transfer units as cabled communication may be desirable in some settings. The output unit 2419 may be such as related to computer software, mobile application, or a central data storage software.

The analyzer unit 2416 of the remote processing unit 2415 is capable of simultaneous analysis of multiple continuous pressure signals 2405 from numerous pressure sensors 2401. The database 2439 may incorporate analysis output from multiple continuous pressure signals 2405. The common approach is to utilize one extension unit 2403 for each pressure transducer system. This does not exclude the opportunity for applying one extension unit for several pressure transducer systems and monitors, e.g. to be sampled continuously in succession.

Aspects of the system 2400 may be evolved from measuring of intracranial pressure (ICP) and/or arterial blood pressure (ABP) signals. Other pressures such as central or peripheral venous pressure, cerebrospinal fluid pressure may utilize the same system 2400. Accordingly, the pressure sensor 2401 may be configured to measure ICP and/or ABP.

The analyzer unit 2416 of the remote processing unit 2415 in the system 2400 utilizes a first type of selectable thresholds 2428 that relates to pressure ranges of dSW.x 2426, a second type of selectable thresholds 2433 that relate to pressure ranges of SW.x.PSL.PD 2430 of various durations (SW.x.PSL.TD) of the same type of single pressure wave (SW.x)-related parameters 2424, and a third type of selectable thresholds 2434 that relate to ratios for combinations of pressure stability levels (SW.x.PSL) 2428 of different types of single pressure wave (SW.x)-related parameters 2424.

The first 2428, second 2433 and third 2434 type of selectable set thresholds utilized by the system 2400 can be created from previously established measurements stored in the database 2439.

The presentation unit 2431 of the analyzer unit 2416 of the system 2400 may be configured to issue an alert if parameters of i) 2433 and/or ii) 2434 are outside respective thresholds. The alert can be at least one of: a warning color of at least one part of the baseline pressure indicator plot 2432 shown on the output unit 2419, and a descriptive information displayed by the output unit 2419.

In some embodiments, the output unit 2419 can be integrated in operation with the remote processing unit 2415 and its analyzer unit 2416 in a number of ways. For example, when the remote processing unit 2415 is related to a cloud-based software system, the output unit 2419 linked thereto may be a software related to a computer or a mobile application or a central data storage related software.

The output 2420 from the output unit 2419 may provide information about baseline stability levels (SW.x.PSL) 2428, pressure differences between pressure stability levels (SW.xPSL. PD) 2430 and baseline pressure indicator (BPi) plots 2432, and statistical information about occurrence of baseline pressure instability 2435. Information about a pressure sensor 2401 may be such as its pressure sensor serial number, model type and batch number. Additional information may be user information, information about location and country where the pressure recording has been performed, as well as information about indication for pressure monitoring. In addition, output may be derived from a database 2439 that stores information about baseline pressure instability 2435.

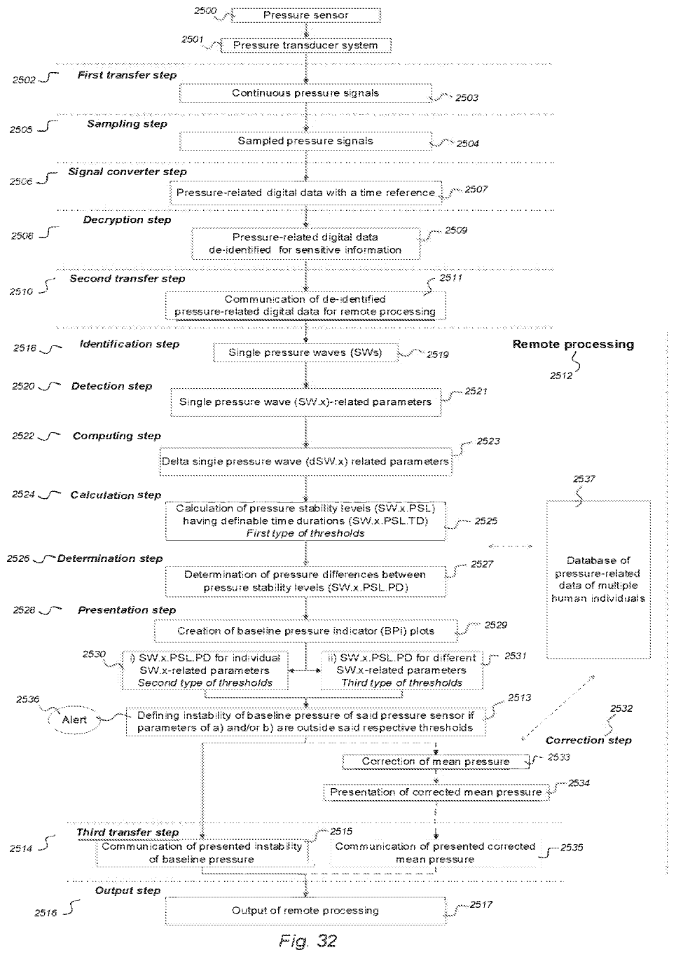

FIG. 32 illustrates a method for assessing stability of baseline pressure of a pressure sensor 2500 being in communication with a pressure transducer system 2501.

The pressure sensor 2500 is capable of measuring continuous pressure signals from inside a human body or body cavity.

The method comprises:
in a first transfer step 2502, receiving, from the pressure sensor 2500, a transfer of continuous pressure signals 2503 measured from inside a human body or a body cavity,
in a sampling step 2505, the continuous pressure signals 2503 are sampled to sampled pressure signals 2504,
in a signal converter step 2506, conversion of the sampled pressure signals 2504 into pressure-related digital data with a time reference 2507, and further
in a decryption step 2508, de-identify the pressure-related digital data with a time reference 2507 for sensitive information, resulting in pressure-related digital data de-identified for sensitive information 2509.

Further, the method comprises
in a second transfer step 2510, transmission or communication 2511 of the pressure-related digital data de-identified for sensitive information 2509 for remote processing 2512.

The second transfer step 2510 is configured to enable remote processing 2512 of the method via dedicated communication.

The remote processing 2512 incorporates analysis of the pressure-related digital data de-identified for sensitive information 2509, and the remote processing 2512 defines instability of baseline pressure 2513 of the pressure sensor 2500. In some embodiments, remote processing 2512 may be performed on a cloud-based software related system or a dedicated stand-alone software system.

In a third transfer step 2514, transmission or communication of information about baseline pressure instability 2515 is enabled.

The method further comprises an output step 2516 that provides for output 2517 of the remote processing, the output 2517 presenting baseline pressure instability of the pressure sensor 2500.

The analysis of the remote processing 2512 comprises
an identification step 2518 to identify, from the de-identified pressure-related digital data 2509, single pressure waves (SWS) 2519 related to cardiac beat-induced pressure waves,
a detection step 2520 to detect, from the single pressure waves, single pressure wave (SW.x)-related parameters 2521, selectable from at least one or more of mean pressure (SW.meanP), and amplitude (SW.dP),
a computing step 2522 to compute one or more of delta single pressure wave (dSW.x)-related parameters 2523, representing differences in single pressure wave (dSW.x)-related parameters and comprising one or more of change in mean pressure (dSW.meanP), and change in amplitude (dSW.dP), between a consecutive number (n−1;n) of single pressure waves (SW.x) 2519,
a calculation step 2524 to calculate pressure stability levels (SW.x.PSL) 2525, each pressure stability level being created of consecutive single pressure waves (SW.x) 2519 having anyone of delta single pressure wave (dSW.x) related parameters dSW.meanP and dSW.dP 2523 within a first set of thresholds 2525, the first set of thresholds referring to defined pressure ranges of anyone of the dSW.x parameters 2523 dSW.meanP and dSW.dP, and wherein a calculated pressure stability level 2525 refers to average of anyone of the single pressure wave (SW.x) related parameters SW.meanP and SW.dP 2523, and
a determination step 2526 to determine pressure differences (SW.x.PSL.PD) 2527 between different (n−1;n) of the pressure stability levels (SW.x.PSL) 2525, wherein the pressure stability levels (SW.x.PSL) have definable time durations (SW.x.PSL.TD) relating to the time duration of the pressure stability levels (SW.x.PSL) 2525, and
a presentation step 2528 to present baseline pressure indicator (BPi) plots 2529, being created from pressure stability levels (SW.x.PSL) 2525 and with beginning pressure differences and ending pressure differences (SW.x.PSL.PD) 2527 for each pressure stability level (SW.x.PSL) 2525, the beginning pressure difference being defined as the difference between a present pressure stability level and a previous pressure stability level and the ending pressure difference being defined as the difference between a present pressure stability level and a next pressure stability level.

The baseline pressure indicator (BPi) plots 2529 provide information about stability of baseline pressure of the pressure sensor 2500 and are a function of at least one of:

i) combinations of the pressure differences between different of the pressure stability levels (SW.x.PSL) 2525, calculated from the same type of single pressure wave (SW.x)-related parameters 2521, the pressure differences being outside or inside a second set of thresholds 2530, reflecting deviations from nominal reference pressure differences, and

ii) relationships between different and simultaneous pressure stability levels (n−1; n) (SW.x.PSL) 2525 calculated from different types of single pressure wave (SW.x)-related parameters 2521, the relationships being outside or inside a third set of thresholds 2531, reflecting deviations from nominal reference relationships.

The presentation step 2528 comprises indicating if parameters of i) 2530 and/or ii) 2531 are outside the respective second set and/or the third set of thresholds and thereby defining 2513 instability of baseline pressure of the pressure sensor 2500.

The single wave mean pressure (SW.meanP) 2519 may in a correction step 2532 undergo correction 2533 of mean pressure as a function of the pressure differences between different of the pressure stability levels (SW.x.PSL.PD) 2527. The corrections 2533 are selectable according to defined criteria, and the corrected mean pressures, based on the correction, are then presented 2534.

One example of criteria for correction of mean pressure (SW.meanP) is given with reference to FIG. 27a-b. Demonstration of the pressure stability levels of mean pressure (SW.meanP.PSL) before 2009 and after 2010 the occurrence of a baseline pressure shift 2007 show that the pressure difference (SW.meanP.PSL.PD) 2011 between the pressure stability levels 2009 and 2010 is >40 mmHg, are thereby outside the criteria. The relationship between SW.meanP.PSL and SW.dP.PSL had an index >10 and also outside the criteria.

The continuous pressure signals 2503 are, in a non-limiting example, intracranial pressure (ICP) and/or arterial blood pressure (ABP) signals, but may include other human pressure signals such as central venous pressure signals, and cerebrospinal fluid pressure signals from lumbar subarachnoid space, the latter are typically characterized as ICP signals. Continuous pressure monitoring of other pressurized locations or cavities in the human body can equally well benefit from use of the present disclosure.

The calculation step 2524 utilizes a first set of thresholds 2525 related to pressure ranges of dSW.x 2523, and the presentation step 2528 utilizes a second set of thresholds 2530 related to pressure difference ranges (SW.x.PSL.PD) 2527 of various durations (SW.x.PSL.TD) of the same type of single pressure wave (SW.x)-related parameters 2521, and a third set of thresholds 2531 related to ratios for combinations of pressure stability levels (SW.x.PSL) 2525 of different types of single pressure wave (SW.x)-related parameters 2521.

The determination step 2526 enables for nearby pressure stability levels (SW.x.PSL) 2525 to merge into one pressure stability level (SW.x.PSL) 2525 if pressure difference between pressure stability levels (SW.x.PSL.PD) 2527 are within selectable ranges of the second set of thresholds 2530.

The presentation step 2528 enables for issuance of an alert 2536 if parameters of i) 2530 and/or ii) 2531 are outside the respective thresholds, the alert 2536 e.g. being at least one of:

a warning color of at least one part of the baseline pressure indicator plot 2529, and

a descriptive information displayed by the presentation step 2528.

The remote processing establishes the first 2525, second 2530 and third 2531 sets of thresholds from previously established measurements stored in a database 2537. The database 2537 contains pressure-related data of multiple human individuals.

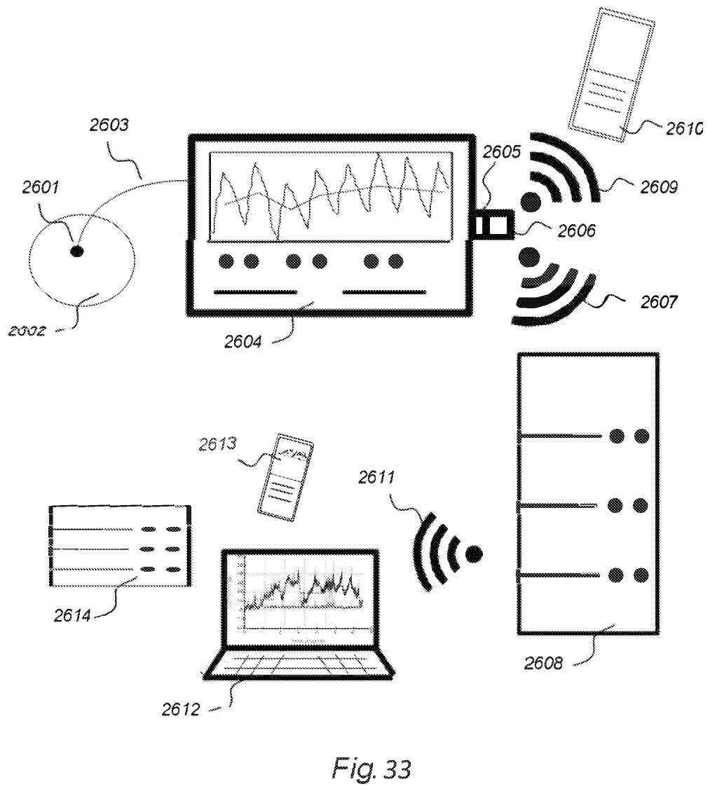

FIG. 33 illustrates a schematic diagram of a pressure sensor 2601 and remote monitoring of data, according to embodiments of the present disclosure. A pressure sensor 2601 is used for measuring pressure within a human body cavity 2602, here illustrated in a non-limiting example by the intracranial cavity. Signals from the sensor 2601 is typically transferred via a cable 2603 to a pressure transducer system 2604. There are several pressure transducer systems 2604 on the market, some examples are listed in Table 17. One example is the CereLink ICP monitor 2604 that is used for monitoring of ICP in humans. The CereLink 2604 is connected via cable 2603 to an ICP sensor 2601 using for measurements of ICP. The CereLink 2604 has a communication port 2605 that enables transfer of continuous ICP signals originating from the ICP sensor 2601.

An extension unit 2606 of the present disclosure may be connected to the communication port 2605 of the pressure transducer 2604, utilizing communication protocols. The size or shape of the extension unit 2606 may be selectable or customized.

The extension unit 2403, 2606 may incorporate a first transfer unit 2404 capable of a first transfer step 2502 enabling transfer of continuous pressure signals from the ICP sensor 2401, 2500, 2601 and pressure transducer system 2402, 2501, 2604 via the communication port 2605 to a sampling unit 2406 (and sampling step 2505) and signal converter 2407 (and signal converter step 2506) to enable conversion of sampled pressure signals 2408, 2504 into pressure-related digital data with a time reference 2409, 2507. A decryption unit 2410 (and decryption step 2508) of the extension unit 2403, 2606 is capable of de-identifying the pressure-related digital data with a time reference 2409, 2507 to pressure-related digital data de-identified for sensitive information 2411, 2509. It should be noted, however, that some pressure transducer systems 2402, 2604 incorporate means for conversion of sampled pressure signals into pressure-related digital data with a time reference 2409, 2507. In these cases, the decryption unit 2410 (and decryption step 2508) of the extension unit 2403, 2606 de-identifies the pressure-related digital data with a time reference 2409, 2507 provided by the pressure transducer system 2402, 2501, 2604 into pressure-related digital data de-identified for sensitive information 2411, 2509. Therefore, whether or not the extension unit 2403, 2606 makes use of or incorporates a signal converter unit 2407 (and signal converter step 2506) depends on the intended pressure transducer system 2402, 2501, 2604. If the signal converter unit 2407 is present, but not useful in view of digital data input thereto, such digital data can e.g. simply be caused to bypass the signal converter unit 2407. In some embodiments, keeping variants of the extension unit to a minimum is an option.

Furthermore, a second transfer unit 2412 (and second transfer step 2510) of the extension unit 2403, 2606 enables for communication 2511, 2607 of de-identified pressure-related digital data 2411, 2509 to a remote processing unit 2415, 2608 (for remote processing 2512). The disclosure is not limited by the specific communication protocol 2607 utilized by the extension unit 2403, 2606. For example, communication 2607 may utilize global system for global communication (GSM) or other communication protocols. Independent of communication protocol 2607 used, the second transfer unit 2412 (and second transfer step 2510) of the extension unit 2403, 2606 enables for off-line 2413 or on-line 2414 communication of pressure-related digital data 2511 to the remote processing unit 2415, 2608 (and remote processing 2512). In most cases, on-line 2414 communication may be used.

Via wireless communication 2609, the extension unit 2403, 2606 may be remotely controlled, e.g. using mobile applications 2610 or other communication platforms. For example, it may be desirable to use a mobile application to control the decryption unit 2410 and decryption step 2508 for de-identification of pressure-related digital data 2411, 2509. What is considered sensitive information varies between countries and geographical regions. For example, the user should be able to define the notation of the pressure-related digital data de-identified for sensitive information 2411, 2509. Moreover, using a mobile application 2610 the user may label the de-identified digital data according to type of pressure sensor and product information about pressure sensor.

The second transfer unit 2412 (and second transfer step 2510) may be part of the extension unit 2403, 2606, although the physical implementation represents no limitation of the disclosure. In some embodiments, the second transfer unit 2412 (and second transfer step 2510) provides for wireless communication 2607, 2609 with the remote processing unit 2415, 2608 (and remote processing 2512), or the mobile application 2610.

The remote processing unit 2415, 2608 enabling for remote processing 2512 may be a cloud-based software related system or a dedicated stand-alone software related system. With reference to feature 4a and 4b of Aspect 4, the remote processing unit 2415, 2608 (and remote processing 2512) comprises an analyzer unit 2416, a database unit 2439, 2537 and a third transfer unit 2417 (and third transfer step 2514). The third transfer unit 2417 (and third transfer step 2514) may utilize various communication protocols, for example GSM, and provide for communication 2611 of analysis output 2418, 2440, 2515, 2535 from the analyzer unit 2416 and the correction unit 2436 to an output unit 2419 (and output step 2516), which is capable of providing an output 2420, 2517 from the remote processing unit 2415, 2608 (and remote processing 2512).

The output unit 2419 enabling the output step 2516 may be dedicated computer related software 2612, or mobile application 2613, or a central data storage related software 2614. Different platforms may be desired depending on intended use.

For example, the output unit 2419, 2612, 2613, 2614 (and output step 2516) of the remote processing unit 2415, 2608 (and remote processing 2512) enables visualization of baseline pressure indicator (BPi) plots 2432, 2529 created from pressure stability levels (SW.x.PSL) 2428, 2525, which defines instability of baseline pressure of the ICP sensor 2435, 2513. The presentation unit 2431 (and presentation step 2528) of the analyzer unit 2416 of the remote processing unit 2416 and remote processing 2512 is configured to indicate whether baseline pressure indicator (BPi) indicator plots define instability of the ICP sensor 2435, 2513. The presentation on output unit 2419, 2612, 2613, 2614 (and output step 2516) of the remote processing unit 2415, 2608 (and remote processing 2512) may also be information about data from database 2439, 2537 about baseline pressure instability from a cohort of patient recordings.

When the remote processing unit 2415, 2608 and remote processing 2512 incorporate a mean pressure correcting unit 2436 (enabling correction step 2532) being connected to an output of the analyzer unit 2416, the mean pressure correcting unit 2436 (enabling correction step 2532) may be configured to correct mean pressure (SW.meanP) levels 2437, 2533 related to baseline pressure instability as a function of the pressure differences between different of the pressure stability levels (SW.x.PSL.PD) 2430, 2527. The corrected mean pressures 2438, 2534 may be visualized by the output unit 2419 (and output step 2516), utilizing different platforms 2612, 2613, or 2614, for communication 2418, 2440, 2515, 2535, 2611 enabled by the third transfer unit 2417 and third transfer step 2514.

The database 2439, 2537 of the remote processing unit 2415, 2608 enabling remote processing 2512 may incorporate analysis output from multiple continuous pressure signals, providing for more optimal determination of the first 2428, 2525 second 2433, 2530 and third 2434, 2531 types of selectable thresholds. Furthermore, pressure-related digital data 2411, 2507 from individual continuous pressure signals 2405, 2503 may be compared with multiple other pressure related data 2411, 2507 from multiple continuous pressure signals 2405, 2503.

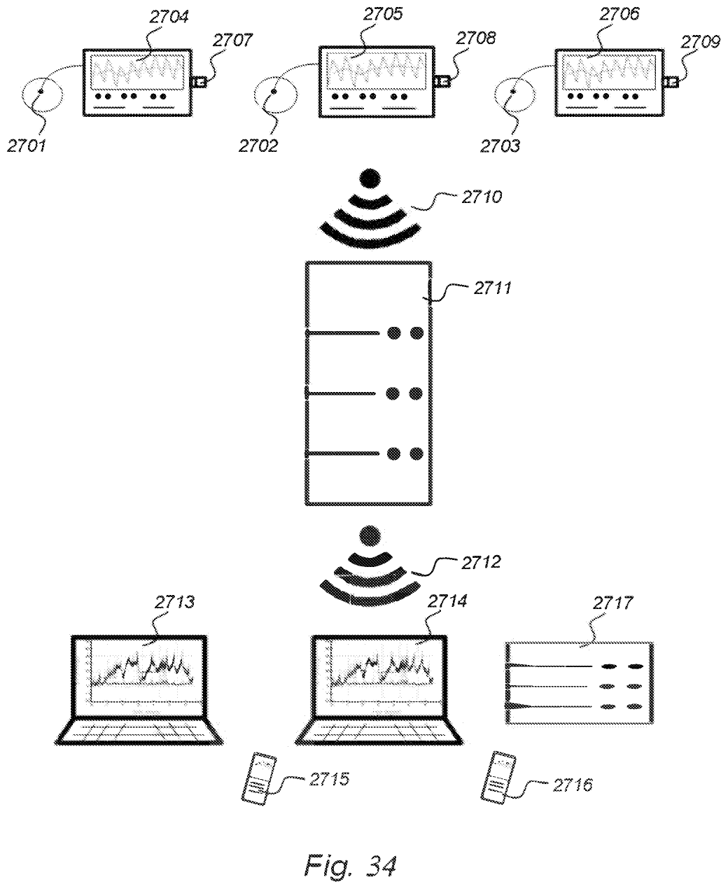

FIG. 34 illustrates a diagram of multiple pressures sensors connected to multiple systems and remote monitoring, according to embodiments of the present disclosure. Different pressure sensors 2701, 2702, 2703 connected to respective pressure transducer systems 2704, 2705, 2706 may via respective extension units 2707, 2708, 2709 of the present disclosure and via dedicated protocol communicate 2710 with a remote processing unit 2711. In this case, there is one extension unit 2707, 2708, 2709 for each pressure transducer system 2704, 2705, 2706. The extension units each enable via a first transfer unit, a signal converter, a decryption unit and a second transfer unit therein, communication 2710 of pressure-related digital data de-identified for sensitive information 2411 from the pressures 2701, 2702, 2703 and pressure transducer systems 2704, 2705, 2706 to the remote processing unit 2711.

The remote processing unit 2711 is capable of simultaneous processing of multiple continuous pressure signals from numerous pressure sensors, here illustrated by sensors 2701, 2702, 2703.

Further, the remote processing unit 2711 comprises the analyzer unit and is capable of providing information about stability of baseline pressure of the pressure sensors 2701, 2702, 2703, being a function of at least one of:

i) combinations of pressure differences between different of the pressure stability levels (SW.x.PSL) 2428, calculated from the same type of single pressure wave (SW.x)-related parameters 2424, the pressure differences being outside or inside a second type of selectable set of thresholds 2433, reflecting deviations from nominal reference pressure differences, and

ii) relationships between different and simultaneous pressure stability levels (n−1; n) 2428 calculated from different types of single pressure wave (SW.x)-related parameters 2424, the relationships being outside or inside a third type of selectable set of thresholds 2434, reflecting deviations from nominal reference relationships. A presentation unit 2431 of the remote processing unit 2711 is configured to indicate if parameters of i) 2433 and/or ii) 2434 are outside the respective thresholds and thereby define 2435 instability of baseline pressure of the pressure sensor 2401.

The remote processing unit 2711 comprises the third transfer unit enabling communication 2712 of output of an analyzer unit of the remote processing unit 2711 to output units. The output units, previously referred to as 2419, may differ and include software installed on various computers 2713, 2714, mobile applications on different mobile units 2715, 2716, or a central data storage related software 2717. This disclosure provides no limitations regarding number of software programs installed on computer 2713, 2714, or number of mobile applications 2715, 2716 or number of central data storage 2717.

There is neither any limitations of this disclosure regarding the infrastructure or location for the remote processing unit 2711 or number of pressure sensors 2701, 2702, 2703 or pressure transducer systems 2704, 2705, 2706 that via extension units 2707, 2708, 2709 may be connected to the remote processing unit 2711. Neither is there any limitation regarding number of output units in communication 2712 with the remote processing unit 2711.

Some examples of implementations are given. In one setting, a manufacturer of pressure sensors may establish a remote processing unit 2711, enabling surveillance of any failure of pressure sensors 2701, 2702, 2703 causing baseline pressure instability. Baseline pressure instability may be related to malfunction of pressure sensors and/or pressure transducer systems caused by manufacturing errors. Therefore, it may be desirable to assess the frequency and severity of baseline pressure instability. For example, certain production batches of pressure sensors could be accompanied by a production failure, causing pressure sensor dysfunction. Today, there are no means for such kind of product surveillance, which is an obvious disadvantage from a patient safety point of view. Moreover, regional differences across countries and even continents related to variations in altitude, atmospheric pressure, temperature, and humidity might be related to pressure sensor failure. Centralized surveillance of baseline pressure instability could help detect such errors, which is enabled by the present disclosure.

Surveillance of pressure sensors 2701, 2702, 2703 and pressure transducer systems 2704, 2705, 2706 may be organized as such. Users of pressure sensors and pressure transducer systems from a certain manufacturer may be provided with extension units 2707, 2708, 2709 enabling communication 2710 of pressure-related digital data de-identified for sensitive information 2411 from the pressures 2701, 2702, 2703 and pressure transducer systems 2704, 2705, 2706 to the remote processing unit 2711. The users may via output units 2713, 2714, 2715, 2716, 2717 obtain information about baseline pressure instability, in addition to the manufacturer being informed by baseline pressure instability.

Today, it is not possible with surveillance of baseline pressure instability while the sensor is within the body of a patient, and which therefore represents a patient safety issue. With the present disclosure, manufacturers of pressures sensors are given opportunity for continuous surveillance of baseline pressure instability, which is both cost saving, convenient, easy to handle, and enables quick response to end users.

Another setting may be research. For example, a study is performed regarding the efficacy of a treatment regimen on ICP. The participants of the study would like to have information about occurrence and severity of baseline pressure instability, which erroneously affect the measured ICP. In such a situation, the remote processing unit 2711 might be in communication 2712 with central data storage related software 2717. Using this approach, the participants may obtain improved control of the measured ICP.

Still another setting might be a consortium of hospitals having different locations wherein patients are undergoing pressure monitoring. To obtain better control of occurrence and severity of baseline pressure instability, centralized analysis of baseline pressure instability might be desired. For this purpose, the hospital could established a remote processing unit in communication with external units via the second transfer unit, and providing information to output units via the third transfer unit. Thereby, it is possible to obtain surveillance of baseline pressure instability in distance from the location where the measurements are being performed. Such technical solutions are presently not available. Aspects of the present disclosure provide technical solutions that enable centralized surveillance of baseline pressure instability, which provides a technical solution for manufacturers of pressure sensors and/or pressure transducer systems to monitor the proper function of their product.

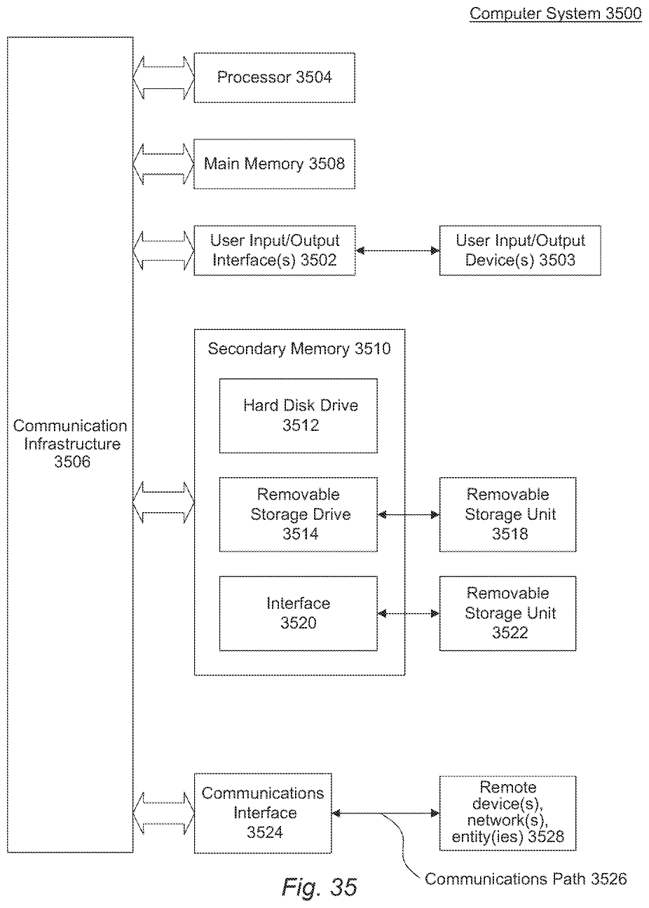

FIG. 35 is a block diagram of example components of computer system 3500, according to embodiments of the present disclosure. One or more computer systems 3500 may be used, for example, to implement any of the embodiments discussed herein, as well as combinations and sub-combinations thereof. In some embodiments, one or more computer systems 3500 may be used to perform the processing, determining, calculating, and other methods used for assessing and correcting baseline pressure instability of pressure sensors, as described herein. Computer system 3500 may include one or more processors (also called central processing units, or CPUs), such as a processor 3504. Processor 3504 may be connected to a communication infrastructure or bus 3506.

Computer system 3500 may also include user input/output interface(s) 3502, such as monitors, keyboards, pointing devices, etc., which may communicate with communication infrastructure 3506 through user input/output device(s) 3503.

One or more of processors 3504 may be a graphics processing unit (GPU). In some embodiments, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 3500 may also include a main or primary memory 3508, such as random access memory (RAM). Main memory 3508 may include one or more levels of cache. Main memory 3508 may have stored therein control logic (i.e., computer software) and/or data. In some embodiments, main memory 3508 may include optical logic configured to perform sepsis detection, sepsis likelihood prediction, pathogen identification, and susceptibility testing, and generate recommendations for treatment of patients accordingly.

Computer system 3500 may also include one or more secondary storage devices or memory 3510. Secondary memory 3510 may include, for example, a hard disk drive 3512 and/or a removable storage drive 3514.

Removable storage drive 3514 may interact with a removable storage unit 3518. Removable storage unit 3518 may include a computer usable or readable storage device having stored thereon computer software (control logic) and/or data. Removable storage unit 3518 may be a program cartridge and cartridge interface (such as that found in video game devices), a removable memory chip (such as an EPROM or PROM) and associated socket, a memory stick and USB port, a memory card and associated memory card slot, and/or any other removable storage unit and associated interface. Removable storage drive 3514 may read from and/or write to removable storage unit 3518.

Secondary memory 3510 may include other means, devices, components, instrumentalities or other approaches for allowing computer programs and/or other instructions and/or data to be accessed by computer system 3500. Such means, devices, components, instrumentalities or other approaches may include, for example, a removable storage unit 3522 and an interface 3520. Examples of the removable storage unit 3522 and the interface 3520 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 3500 may further include a communication or network interface 3524. Communication interface 3524 may enable computer system 3500 to communicate and interact with any combination of external devices, external networks, external entities, etc. (individually and collectively referenced by reference number 3528). For example, communication interface 3524 may allow computer system 3500 to communicate with external or remote devices 3528 over communications path 3526, which may be wired and/or wireless (or a combination thereof), and which may include any combination of LANs, WANs, the Internet, etc. Control logic and/or data may be transmitted to and from computer system 3500 via communication path 3526.

Computer system 3500 may also be any of a personal digital assistant (PDA), desktop workstation, laptop or notebook computer, netbook, tablet, smartphone, smartwatch or other wearables, appliance, part of the Internet-of-Things, and/or embedded system, to name a few non-limiting examples, or any combination thereof

Computer system 3500 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 3500 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 3500, main memory 3508, secondary memory 3510, and removable storage units 3518 and 3522, as well as tangible articles of manufacture embodying any combination of the foregoing. Such control logic, when executed by one or more data processing devices (such as computer system 3500), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 35. In particular, embodiments can operate with software, hardware, and/or operating system implementations other than those described herein.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

Embodiments of the present disclosure have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

### APPENDIX A—ABBREVIATIONS

ABP=Arterial blood pressure
AUC=Area under curve
BPE=Baseline pressure error
BPI=Baseline pressure instability
CSF=cerebrospinal fluid
CSFP=cerebrospinal fluid pressure
CPP=Cerebral perfusion pressure
dP=amplitude

### dSW.AUC=

dSW.dP=difference in single wave amplitude
dSW.meanP=difference in single wave mean pressure
dSW.RT=difference in single wave rise time
dSW.RTC=difference in single wave rise time coefficient
dSW.WD=difference in single wave duration
dSW.x=difference in single pressure wave parameter
RT=Rise time
EVD=External ventricular drainage
GSM=global system for global communication
IAAC=intracranial pressure amplitude arterial blood pressure amplitude correlation
ICP=Intracranial pressure
MeanP=Mean pressure
MWA=mean wave amplitude
mmHg=millimeter mercury

### Pa=Pascal

P_{C}=pressure within a cavity
P_{REF}=reference pressure P_{M}=measured pressure P₀=baseline pressure
PRx=Pressure reactivity index
PSL=pressure stability level
RAP=Relationship amplitude pressure
BPI=Baseline pressure instability
BPi Plot=Baseline pressure indicator plot
RTC=Rise time coefficient
SW=single pressure wave
SW.AUC=single wave area under curve
SW.dP=single wave amplitude
SW.meanP=single wave mean pressure
SW.dP.PSL=single wave amplitude pressure stability level
SW.dP.PSL.PD=pressure difference between single wave amplitude pressure stability levels
SW.meanP.PSL=single wave pressure stability level for mean pressure
SW.MeanP_{Corr}=single wave corrected mean pressure
SW.RTC.PSL=single wave pressure stability level for rise time coefficient
SW.meanP.PSL.PD=pressure difference between pressure stability levels for mean pressure
SW.x.PSL=single wave pressure stability level
SW.x.PSL.PD=pressure difference between pressure stability levels
SW.meanP.PSL.PD=pressure difference between pressure stability levels for single wave mean pressure
SW.dP.PSL.PD=pressure difference between pressure stability levels for single wave amplitude
SW.RTC.PSL.PD=pressure difference between pressure stability levels for rise time coefficient
SW.x.PSL.TD=single wave pressure stability level time duration
SW.meanP.PSL.TD=single wave pressure stability level time duration for single wave mean pressure
SW.dP.PSL.TD=single wave pressure stability level time duration for single wave amplitude
SW.RTC.PSL.TD=single wave pressure stability level time duration for single wave rise time coefficient
SW.RT=single wave rise time
SW.RTC=single wave rise time coefficient
SW.x=single wave parameter
SW.WD=single wave duration
WD=Wave duration

What is claimed is:

1. A system for assessing stability of baseline pressure of a pressure sensor in communication with a pressure transducer system and configured to measure continuous pressure signals from inside a human body or body cavity, the system comprising:
an extension unit;
a remote processing unit; and
an output unit,
wherein the extension unit comprises:
a first transfer unit configured to transfer continuous pressure signals from the pressure sensor to a sampling unit;
a signal converter in communication with the sampling unit and configured to perform conversion of sampled continuous pressure signals into pressure-related digital data with a time reference;
a decryption unit, configured to de-identify the pressure-related digital data for sensitive information, resulting in de-identified pressure-related digital data; and
a second transfer unit configured to transmit the de-identified pressure-related digital data to the remote processing unit,
wherein the remote processing unit comprises:
an analyzer unit configured to analyze the de-identified pressure-related digital data; and
a third transfer unit configured to transmit an analysis output from the analyzer unit to the output unit, wherein the output unit is configured to provide an output of the remote processing unit, the output presenting baseline pressure instability of a pressure sensor.

2. The system of claim 1, wherein the analyzer unit of the remote processing unit further comprises:
an identifier unit configured to receive the de-identified pressure-related digital data from the second transfer unit and identify single pressure waves related to cardiac beat-induced pressure waves from the de-identified pressure-related digital data;
a detector connected to an output of the identifier unit and configured to detect single pressure wave (SW.x)-related parameters from the single pressure waves, being at least one or more of mean pressure (SW.meanP) and amplitude (SW.dP); and
a computing device connected to an output of the detector and configured to compute one or more of delta single pressure wave (dSW.x)-related parameters representing differences in single pressure wave (dSW.x)-related parameters being one or more of change in mean pressure (dSW.meanP), and change in amplitude (dSW.dP), between a consecutive number (n−1;n) of single pressure waves (SW.x),
wherein a calculation unit is connected to an output of the computing device and configured to calculate pressure stability levels (SW.x.PSL), each pressure stability level being created from consecutive single pressure waves having any one of the delta single pressure wave (dSW.x)-related parameters dSW.meanP and dSW.dP within a first set of thresholds, the first set of thresholds referring to defined pressure ranges of any one of the parameters dSW.meanP and dSW.dP, wherein each pressure stability level (SW.x.PSL) refers to an average of any one of the single pressure wave (SW.x)-related parameters SW.meanP and SW.dP,
wherein a determination unit is connected to an output of the calculation unit and configured to determine pressure differences (SW.x.PSL.PD) between different pressure stability levels (n−1;n) (SW.x.PSL),
wherein the pressure stability levels (SW.x.PSL) have definable time durations (SW.x.PSL.TD) relating to a time duration of the pressure stability levels (SW.x.PSL),
wherein a presentation unit is connected to an output of the determination unit and configured to present baseline pressure indicator (BPi) plots, being created from pressure stability levels (SW.x.PSL) and with beginning pressure differences and ending pressure differences (SW.x.PSL.PD) for each pressure stability level (SW.x.PSL), the beginning pressure difference being defined as a difference between a present pressure stability level and a previous pressure stability level and the ending pressure difference being defined as a difference between a present pressure stability level and a next pressure stability level,
wherein the BPi plots provide information about stability of baseline pressure of the pressure sensor and are a function of at least one of:
i) combinations of the pressure differences between different pressure stability levels (n−1; n) (SW.x.PSL), calculated from a same type of single pressure wave (SW.x)-related parameters, the pressure differences being outside or inside a second set of thresholds, reflecting deviations from nominal reference pressure differences, and
ii) relationships between different and simultaneous pressure stability levels (n−1; n) (SW.x.PSL) calculated from different types of single pressure wave (SW.x)-related parameters, the relationships being outside or inside a third set of thresholds, reflecting deviations from nominal reference relationships, and
wherein the presentation unit is configured to indicate if parameters of i) and/or ii) are outside the second set and/or the third set of thresholds and thereby define instability of baseline pressure of the pressure sensor.

3. The system of claim 2, wherein a correction unit is connected to an output of the analyzer unit of the remote processing unit and configured to correct mean pressure (SW.meanP) levels related to the baseline pressure instability as a function of the pressure differences between different pressure stability levels (SW.x.PSL.PD), the correction of mean pressure levels being selectable according to predefined criteria, and wherein the correction unit is configured to present the corrected mean pressure.

4. The system of claim 3, wherein an output from the correction unit is in communication with the output unit via the third transfer unit.

5. The system of claim 1, wherein the second transfer unit provides wireless communication of the de-identified pressure-related digital data to the remote processing unit.

6. The system of claim 1, wherein the remote processing unit is a cloud-based software system or a dedicated stand-alone software system.

7. The system of claim 1, wherein the third transfer unit is configured to route the analysis output from the analyzer unit to a database unit, and to a correction unit, before transmitting the analysis output to the output unit.

8. The system of claim 7, wherein the correction unit is coupled to the database unit.

9. The system of claim 2, wherein the analyzer unit of the remote processing unit is configured to perform a simultaneous analysis of multiple continuous pressure signals from a plurality of pressure sensors and pressure transducer systems, and wherein a database is configured to store output data derived from the simultaneous analysis of the multiple continuous pressure signals.

10. The system of claim 2, wherein the analyzer unit utilizes the first set of thresholds to relate to pressure ranges of dSW.x, the second set of thresholds to relate to pressure ranges of SW.x.PSL.PD of various time durations (SW.x.PSL.TD) of the same type of single pressure wave (SW.x)-related parameters, and the third set of thresholds to relate to ratios for combinations of pressure stability levels (SW.x.PSL) of different types of single pressure wave (SW.x)-related parameters.

11. The system of claim 2, wherein the first, second and third sets of thresholds are created from previously established measurements stored in a database.

12. The system of claim 1, wherein the remote processing unit is configured to perform a surveillance of the stability of baseline pressure of a plurality of pressure sensors simultaneously.

13. A method for assessing stability of baseline pressure of a pressure sensor in communication with a pressure transducer system, the method comprising:
receiving, from the pressure sensor, continuous pressure signals measured from inside a human body or a body cavity;
sampling, by one or more computing devices, the continuous pressure signals to sampled continuous pressure signals;
converting, by the one or more computing devices, the sampled continuous pressure signals into pressure-related digital data with a time reference;
de-identifying, by the one or more computing devices, the pressure-related digital data for sensitive information, resulting in de-identified pressure-related digital data;
transmitting, by the one or more computing devices, the de-identified pressure-related digital data for remote processing, the remote processing comprising an analysis of the de-identified pressure-related digital data and an identification of a baseline pressure instability of the pressure sensor;
transmitting, by the one or more computing devices, information about the baseline pressure instability; and
providing, by the one or more computing devices, an output of the remote processing, the output presenting the baseline pressure instability of the pressure sensor.

14. The method of claim 13, wherein the analysis of the remote processing comprises:
identifying, from the de-identified pressure-related digital data, single pressure waves related to cardiac beat-induced pressure waves,
detecting, from the single pressure waves, single pressure wave (SW.x)-related parameters, selectable from one or more of mean pressure (SW.meanP) and amplitude (SW.dP),
computing one or more of delta single pressure wave (dSW.x)-related parameters, representing differences in single pressure wave (dSW.x)-related parameters and comprising one or more of a change in mean pressure (dSW.meanP), and a change in pressure amplitude (dSW.dP), between a consecutive number of single pressure waves (n−1;n),
calculating pressure stability levels (SW.x.PSL), each pressure stability level being created from consecutive single pressure waves having any one of the delta single pressure wave (dSW.x)-related parameters dSW.meanP and dSW.dP within a first set of thresholds, the first set of thresholds referring to defined pressure ranges of any one of the parameters dSW.meanP and dSW.dP, and wherein each pressure stability level refers to an average of any one of the single pressure wave (SW.x)-related parameters SW.meanP and SW.dP,
determining pressure differences (SW.x.PSL.PD) between different pressure stability levels (n−1;n) (SW.x.PSL), wherein the pressure stability levels (SW.x.PSL) have definable time durations (SW.x.PSL.TD) relating to a time duration of the pressure stability levels (SW.x.PSL), and
presenting baseline pressure indicator (BPi) plots being created from pressure stability levels (SW.x.PSL) and with beginning pressure difference and ending pressure differences (SW.x.PSL.PD) for each pressure stability level (SW.x.PSL), the beginning pressure difference being defined as the difference between a present pressure stability level and a previous pressure stability level, and the ending pressure difference being defined as the difference between a present pressure stability level and a next pressure stability level,
wherein the BPi plots provide information about the stability of baseline pressure of the pressure sensor and are a function of at least one of:
i) combinations of the pressure differences between different pressure stability levels (SW.x.PSL), calculated from a same type of single pressure wave (SW.x)-related parameters, the pressure differences being outside or inside a second set of thresholds, reflecting deviations from nominal reference pressure differences, and
ii) relationships between different and simultaneous pressure stability levels (n−1; n) (SW.x.PSL) calculated from different types of single pressure wave (SW.x)-related parameters, the relationships being outside or inside a third set of thresholds, reflecting deviations from nominal reference relationships, and
wherein the presenting of the BPi plots comprises indicating if parameters of i) and/or ii) are outside the second set and/or the third set of thresholds and thereby defining the baseline pressure instability of the pressure sensor.

15. The method of claim 14, wherein the single wave mean pressure (SW.meanP) is undergoing correction as a function of the pressure differences between the different pressure stability levels (SW.x.PSL.PD), the correction being selectable according to defined criteria, and wherein corrected mean pressures based on the correction are presented.

16. The method of claim 14, wherein the remote processing establishes the first, second and third sets of thresholds from previously established measurements stored in a database.

17. The method of claim 13, wherein the remote processing is performed on a cloud-based software system or a dedicated stand-alone software related system.

18. The method of claim 14, wherein the first set of thresholds is related to pressure ranges of dSW.x, the second set of thresholds is related to pressure ranges of SW.x.PSL.PD of various time durations (SW.x.PSL.TD) of the same type of single pressure wave (SW.x)-related parameters, and the third set of thresholds is related to ratios for combinations of pressure stability levels (SW.x.PSL) of different types of single pressure wave (SW.x)-related parameters.

19. The method of claim 13, wherein the remote processing comprises assessing stability of baseline pressure of a plurality of pressure sensors simultaneously.

20. The method of claim 15, wherein the corrected mean pressures are communicated during the transmission of the information about the baseline pressure instability by the one or more computing devices, and the corrected mean pressures are presented in the output of the remote processing.

21. The method of claim 15, further comprising: communicating, by the one or more computing devices, with a database storing pressure-related data of multiple individuals, for the analysis in the remote processing and the correction of the single wave mean pressure.
